(19) United States
(12) Patent Application Publication
(10) Pub. No.: US 2023/0374455 A1
(43) Pub. Date: Nov. 23, 2023

(54) T CELL MANUFACTURING COMPOSITIONS AND METHODS

(71) Applicant: BioNTech US Inc., Cambridge, MA (US)

(72) Inventors: Vikram Juneja, Newton, MA (US); Jared Dietze, Norwood, MA (US); Brandon Conn, Somerville, MA (US); Jaewon Choi, Jamaica Plain, MA (US)

(21) Appl. No.: 18/007,024

(22) PCT Filed: Aug. 12, 2021
(86) PCT No.: PCT/US21/45800

§ 371 Date: Jan. 26, 2023

Related U.S. Application Data
(60) Provisional application No. 63/065,327, filed on Aug. 13, 2020.

(51) Int. Cl. C12N 5/0783; A61K 39/00

(52) U.S. Cl. CPC C12N 5/0636; A61K 39/4611; A61K 39/4644; A61K 39/4634; C12N 2502/1121

(57) ABSTRACT

The generation of antigen specific T cells by controlled ex vivo induction or expansion can provide highly specific and beneficial T cell therapies. The present disclosure provides T cell manufacturing methods and therapeutic T cell compositions which can be used for treating subjects with cancer and other conditions, diseases and disorders personal antigen specific T cell therapy.

### CROSS-REFERENCE

This application is the National Stage Entry of the PCT application, PCT/US21/45800 filed on Aug. 12, 2021, which claims the benefit of U.S. Provisional Application No. 63/065,327, filed on Aug. 13, 2020; both of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 28, 2021, is named 50401-754_601_SL.txt and is 50,700 bytes in size.

### BACKGROUND

Tumor vaccines are typically composed of tumor antigens and immunostimulatory molecules (e.g., adjuvants, cytokines or TLR ligands) that work together to induce antigen-specific cytotoxic T cells (CTLs) that recognize and lyse tumor cells. Such vaccines contain either shared tissue restricted tumor antigens or a mixture of shared and patient-specific antigens in the form of whole tumor cell preparations. The shared tissue restricted tumor antigens are ideally immunogenic proteins with selective expression in tumors across many individuals and are commonly delivered to patients as synthetic peptides or recombinant proteins. In contrast, whole tumor cell preparations are delivered to patients as autologous irradiated cells, cell lysates, cell fusions, heat-shock protein preparations or total mRNA. Since whole tumor cells are isolated from the autologous patient, the cells may include patient-specific tumor antigens as well as shared tumor antigens. Finally, there is a third class of tumor antigens, neoantigens, that has rarely been used in vaccines, which consists of proteins with tumor-specific mutations (which can be patient-specific or shared) that result in altered amino acid sequences. Such mutated proteins are: (a) unique to the tumor cell as the mutation and its corresponding protein are present only in the tumor; (b) avoid central tolerance and are therefore more likely to be immunogenic; (c) provide an excellent target for immune recognition including by both humoral and cellular immunity.

Adoptive immunotherapy or adoptive cellular therapy (ACT) is the transfer of lymphocytes to a subject for the therapy of disease. Adoptive immunotherapy has yet to realize its potential for treating a wide variety of diseases including cancer, infectious disease, autoimmune disease, inflammatory disease, and immunodeficiency. However, most, if not all adoptive immunotherapy strategies require T cell activation and expansion steps to generate a clinically effective, therapeutic dose of T cells. Due to the inherent complexity of live cell culture and patient to patient variability, current technologies for generating therapeutic doses of T cells, including engineered T cells, remain limited by cumbersome T cell manufacturing processes. Existing T cell manufacturing processes are not easily scalable, repeatable, reliable, or efficient and often produce an inferior T cell product that may be prone to exhaustion and loss of effector immune cell function. To date, engineered T cell adoptive immunotherapies have met with only limited success and routinely show variable clinical activity. Therefore, such therapies are not suitable for widespread clinical use. Accordingly, there remains a need for developing compositions and methods for expansion and induction of antigen specific T cells with a favorable phenotype and function.

To date, administration of a successful cell therapy relies on large numbers of antigen-specific T cells. Unfortunately, not all epitopes yield high amounts of antigen-specific cells after ex vivo stimulation. Therefore, enrichment and subsequent expansion of antigen-specific T cells may be necessary to achieve an effective product.

### SUMMARY

Provided herein is a method for producing a therapeutic population of T cells comprising: (a) culturing T cells from a biological sample from a subject in a first cell culture medium comprising antigen presenting cells (APCs) to produce a first population of T cells, wherein the APCs present an epitope of a peptide antigen in complex with an MHC protein; (b) optionally, culturing the first population of T cells in a second cell culture medium to produce a second population of T cells; (c) enriching CD137 (4-1BB)-expressing T cells and/or CD69 expressing T cells from the first or second population of T cells to produce a third population of T cells; and (d) expanding the third population of T cells in a third cell culture medium to obtain a therapeutic population of T cells comprising antigen-specific T cells.

In some embodiments, the method comprises culturing the first population of T cells in a second cell culture medium to produce the second population of T cells, and enriching CD137 (4-1BB)-expressing T cells and/or CD69 expressing T cells from the second population of T cells to produce the third population of T cells.

Provided herein is a method for producing a therapeutic population of T cells comprising: (a) culturing T cells from a biological sample from a subject in a first cell culture medium comprising antigen presenting cells (APCs), wherein the APCs present an epitope of a peptide antigen in complex with an MHC protein; (b) culturing the first population of T cells in a second cell culture medium to produce a second population of T cells; (c) optionally, enriching CD137 (4-1BB)-expressing T cells and/or CD69 expressing T cells from the second population of T cells to produce a third population of T cells; and (d) expanding the second or third population of T cells in a third cell culture medium to obtain a therapeutic population of T cells comprising antigen-specific T cells; wherein the concentration of the peptide antigen in the third culture medium is at least 2-fold lower than the concentration of the peptide antigen in the first culture medium and/or second culture medium.

In some embodiments, the method comprises enriching CD137 (4-1BB)-expressing T cells and/or CD69 expressing T cells from the second population of T cells to produce a third population of T cells.

In some embodiments, enriching CD137 (4-1BB)-expressing T cells and/or CD69 expressing T cells from the second population of T cells begins 10, 11, 12, 13, 14, 15, 16, 17 or 18 days after beginning the culturing of the T cells from a biological sample from a subject in a first cell culture medium.

In some embodiments, the APCs (i) comprise a polynucleotide sequence encoding the peptide antigen, or (ii) are loaded with the epitope of a peptide antigen.

In some embodiments, the peptide antigen is directly added to the first cell culture medium.

In some embodiments, the first cell culture medium comprises a first concentration of the peptide antigen

In some embodiments, the method further comprises supplementing the first cell culture medium with an amount of the peptide antigen such that the first cell culture medium comprises a first concentration of the peptide antigen.

In some embodiments, the first concentration of the peptide antigen is from 1 nM to 100 μM or from 100 nM to 10 μM.

In some embodiments, the first concentration of the peptide antigen is about 1 μM, 2 μM, 3 μM, 4 μM or 5 μM.

In some embodiments, the second cell culture medium comprises a second concentration of the peptide antigen

In some embodiments, the method further comprises supplementing the second cell culture medium with an amount of the peptide antigen such that the second cell culture medium comprises a second concentration of the peptide antigen.

In some embodiments, the second concentration of the peptide antigen is higher, lower or about the same as the first concentration of the peptide antigen.

In some embodiments, the second concentration of the peptide antigen is from 1 nM to 100 μM or from 100 nM to 10 μM.

In some embodiments, second concentration of the peptide antigen is about 1 μM, 2 μM, 3 μM, 4 μM or 5 μM.

In some embodiments, culturing the first population of T cells in the second cell culture medium begins 9, 10, 11, 12, 13, 14, 15, 16 or 17 days after beginning the culturing of the T cells from a biological sample from a subject in a first cell culture medium.

In some embodiments, the third cell culture medium comprises a third concentration of the peptide antigen.

In some embodiments, the method further comprises supplementing the third cell culture medium with an amount of the peptide antigen such that the third cell culture medium comprises a third concentration of the peptide antigen.

In some embodiments, the third concentration of the peptide antigen is at least 2-fold lower than the first concentration of the peptide antigen.

In some embodiments, the third concentration of the peptide antigen is at least 2-fold lower than the second concentration of the peptide antigen.

In some embodiments, the third concentration of the peptide antigen is at least 3, 4, 5, 6, 7, 8, 9 or 10-fold lower than the first concentration of the peptide antigen.

In some embodiments, the third concentration of the peptide antigen is at least 3, 4, 5, 6, 7, 8, 9 or 10-fold lower than the second concentration of the peptide antigen.

In some embodiments, the third concentration of the peptide antigen is from 0.1 nM to 10 μM.

In some embodiments, the third concentration of the peptide antigen is about 0.1 nM, 0.5, nM, 1 nM, 10 nM, 25 nM, 50 nM, 100 nM, 150 nM, 200 nM, 300 nM, 400 nM, 500 nM, 1 μM or 10 μM.

In some embodiments, expanding the second or third population of T cells in the third cell culture medium begins 11, 12, 13, 14, 15, 16, 17, 18 or 19 days after beginning the culturing of the T cells from a biological sample from a subject in a first cell culture medium

In some embodiments, expanding the second or third population of T cells in the third cell culture medium begins 1, 2, 3 4 or 5 days after enriching CD137 (4-1BB)-expressing T cells and/or CD69 expressing T cells from the second population of T cells.

In some embodiments, expanding the second or third population of T cells in a third cell culture medium comprises expanding the second or third population of T cells in the presence of an increasing concentration of the peptide antigen.

In some embodiments, expanding the second or third population of T cells in the presence of an increasing concentration of the peptide antigen comprises expanding the second or third population of T cells in a fourth cell culture medium comprising a fourth concentration of the peptide antigen

In some embodiments, expanding the second or third population of T cells in the presence of an increasing concentration of the peptide antigen comprises supplementing the third cell culture medium with an amount of the peptide antigen such that the third cell culture medium comprises a fourth concentration of the peptide antigen, wherein the fourth concentration of the peptide antigen is at least 1.1-fold higher than the third concentration of the peptide antigen.

In some embodiments, the fourth concentration of the peptide antigen is at least 2, 3, 4, 5, 6, 7, 8, 9 or 10-fold higher than the third concentration of the peptide antigen.

In some embodiments, the fourth concentration of the peptide antigen is from 1 nM to 50 μM.

In some embodiments, the fourth concentration of the peptide antigen is about 1 nM, 10 nM, 25 nM, 50 nM, 100 nM, 150 nM, 200 nM, 300 nM, 400 nM, 500 nM, 600 nM, 700 nM, 800 nM, 900 nM, 1 μM, 10 μM, 25 μM or 50 μM.

In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 12, 13, 14, 15, 16, 17, 18, 19 or 20 days after beginning the culturing of the T cells from a biological sample from a subject in a first cell culture medium.

In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 1, 2, 3, 4 or 5 days after beginning expanding the second or third population of T cells in a third cell culture medium comprising a third concentration of the peptide antigen or 1, 2, 3, 4 or 5 days after supplementing the third cell culture medium with an amount of the peptide antigen such that the third cell culture medium comprises a third concentration of the peptide antigen.

In some embodiments, expanding the second or third population of T cells in the presence of an increasing concentration of the peptide antigen comprises expanding the second or third population of T cells in a fifth cell culture medium comprising a fifth concentration of the peptide antigen

In some embodiments, expanding the second or third population of T cells in the presence of an increasing concentration of the peptide antigen comprises supplementing the fourth cell culture medium with an amount of the peptide antigen such that the fourth cell culture medium comprises a fifth concentration of the peptide antigen, wherein the fifth concentration of the peptide antigen is at least 1.1-fold higher than the fourth concentration of the peptide antigen.

In some embodiments, the fifth concentration of the peptide antigen is at least 2, 3, 4, 5, 6, 7, 8, 9 or 10-fold higher than the third concentration of the peptide antigen and/or the fourth concentration of the peptide antigen.

In some embodiments, the fifth concentration of the peptide antigen is from 10 nM to 100 μM.

In some embodiments, the fifth concentration of the peptide antigen is about 10 nM, 25 nM, 50 nM, 100 nM, 150 nM, 200 nM, 300 nM, 400 nM, 500 nM, 600 nM, 700 nM, 800 nM, 900 nM, 1 μM, 10 μM, 25 μM, 50 μM, 75 μM or 100 μM.

In some embodiments, expanding the second or third population of T cells in a fifth cell culture medium comprising a fifth concentration of the peptide antigen or supplementing the fourth cell culture medium with an amount of the peptide antigen such that the fourth cell culture medium comprises a fifth concentration of the peptide antigen begins 13, 14, 15, 16, 17, 18, 19, 20 or 21 days after beginning the culturing of the T cells from a biological sample from a subject in a first cell culture medium.

In some embodiments, expanding the second or third population of T cells in a fifth cell culture medium comprising a fifth concentration of the peptide antigen or supplementing the fourth cell culture medium with an amount of the peptide antigen such that the fourth cell culture medium comprises a fifth concentration of the peptide antigen begins 1, 2, 3, 4 or 5 days after expanding the second or third population of T cells in a fourth cell culture medium comprising a fourth concentration of the peptide antigen or 1, 2, 3 4 or 5 days after supplementing the fourth cell culture medium with an amount of the peptide antigen such that the fourth cell culture medium comprises a fourth concentration of the peptide antigen.

In some embodiments, expanding the second or third population of T cells in a fifth cell culture medium comprising a fifth concentration of the peptide antigen or supplementing the fourth cell culture medium with an amount of the peptide antigen such that the fourth cell culture medium comprises a fifth concentration of the peptide antigen begins 2, 3, 4, 5 or 6 days after expanding the second or third population of T cells in a third cell culture medium comprising a third concentration of the peptide antigen or 2, 3, 4, 5 or 6 days after supplementing the third cell culture medium with an amount of the peptide antigen such that the third cell culture medium comprises a third concentration of the peptide antigen.

In some embodiments, the number of antigen-specific T cells in the second or third population of T cells is greater than the number of antigen-specific T cells in the first population of T cells.

In some embodiments, the frequency of antigen-specific T cells in the second or third population of T cells is greater than the frequency of antigen-specific T cells in the first population of T cells, wherein the frequency of antigen-specific T cells in a population of T cells is the [number of antigen-specific T cells in the population]/[total number of T cells in the population]×100.

In some embodiments, the frequency of antigen-specific T cells in the therapeutic population of T cells is greater than the frequency of antigen-specific T cells in the first population of T cells, wherein the frequency of antigen-specific T cells in a population of T cells is the [number of antigen-specific T cells in the population]/[total number of T cells in the population]×100.

In some embodiments, the frequency of antigen-specific T cells in the therapeutic population of T cells is greater than the frequency of antigen-specific T cells in the second population of T cells, wherein the frequency of antigen-specific T cells in a population of T cells is the [number of antigen-specific T cells in the population]/[total number of T cells in the population]×100.

In some embodiments, the frequency of antigen-specific T cells in the therapeutic population of T cells is greater than the frequency of antigen-specific T cells in the third population of T cells, wherein the frequency of antigen-specific T cells in a population of T cells is the [number of antigen-specific T cells in the population]/[total number of T cells in the population]×100.

In some embodiments, culturing of the first population of T cells is performed for a period of from 5 to 25 days, from 7 to 16 days, from 13 to 15 days or about 13 or 14 days.

In some embodiments, culturing of the second population of T cells is performed for a period of 1, 2, 3, or 4 days.

In some embodiments, culturing of the second population of T cells is performed for a period of from 5 to 25 days, from 7 to 14 days, from 11 to 13 days, 21 days or less, or about 12 days.

In some embodiments, expanding the second or third population of T cells is performed for a period of from 5 to 25 days, from 7 to 14 days, from 11 to 13 days, 21 days or less, or about 12 days.

In some embodiments, expanding the second or third population of T cells is performed for a period of from 4 to 24 days, from 6 to 13 days, from 10 to 12 days, 20 days or less, or about 11 days.

In some embodiments, the method expands antigen-specific T cells.

In some embodiments, the method expands naive T cells from the first population of T cells.

In some embodiments, the method expands naive T cells from the first population of T cells that have become antigen-specific T cells.

In some embodiments, the method comprises expanding antigen-specific T cells.

In some embodiments, culturing T cells from a biological sample from a subject in a first cell culture medium expands antigen-specific T cells.

In some embodiments, culturing the first population of T cells in a second cell culture medium expands antigen-specific T cells.

In some embodiments, expanding the second or third population of T cells in a third cell culture medium expands antigen-specific T cells.

In some embodiments, the first population of T cells is not obtained from a tumor infiltrating lymphocyte (TIL) sample.

In some embodiments, the first and second culture mediums are the same.

In some embodiments, the first and second culture mediums are different.

In some embodiments, the first culture medium comprises GM-CSF, IL-4, FLT3L, TNF-α, IL-1β, PGE1, IL-6, IL-7, IL-12, IFN-α, R848, LPS, ss-rna40, poly I:C, or any combination thereof.

In some embodiments, the second culture medium comprises a soluble anti-CD3 antibody, an anti-CD3 antibody conjugated to a bead, soluble anti-CD28 antibody, an anti-CD28 antibody conjugated to a bead, insulin, one or more non-essential amino acids, glucose, glutamine, IL-2, IL-7, IL-15, IL-12, a CD137 agonist, an AKT inhibitor, a MEM vitamin solution, sodium pyruvate or any combination thereof.

In some embodiments, the first culture medium comprises FMS-like tyrosine kinase 3 receptor ligand (FLT3L).

In some embodiments, the second culture medium comprises FLT3L.

In some embodiments, the second culture medium does not comprise additional APCs.

In some embodiments, a number of APCs present in the second or the third culture medium is less than the number of APCs present in the first cell culture medium.

In some embodiments, supplementing does not comprise supplementing with APCs.

In some embodiments, the method comprises enriching CD137-expressing T cells from the second population of T cells after (a) and before (b).

In some embodiments, enriching comprises enriching with an enriching reagent comprising an anti-CD137 reagent.

In some embodiments, the enriching reagent is an antibody or binding fragment thereof.

In some embodiments, the enriching reagent is coupled to a solid surface.

In some embodiments, enriching comprises immunoprecipitating.

In some embodiments, the second and/or the third culture media is supplemented with a T cell activator.

In some embodiments, the T cell activator comprises soluble CD3 and or CD28 coated beads.

In some embodiments, the method further comprises harvesting the therapeutic population of T cells comprising antigen-specific T cells.

In some embodiments, the method further comprises transferring the harvested therapeutic population of T cells comprising antigen-specific T cells to an infusion bag.

In some embodiments, the method further comprises administering the therapeutic population of T cells comprising antigen-specific T cells to the subject.

In some embodiments, the subject has a disease or condition.

In some embodiments, the disease or condition is cancer.

In some embodiments, the cancer is a solid cancer.

In some embodiments, the cancer is melanoma, pancreatic ductal adenocarcinoma (PDAC), colorectal cancer (CRC) or non-small cell lung cancer (NSCLC).

In some embodiments, the cancer is unresectable melanoma or RAS mutant PDAC.

In some embodiments, the subject is human.

In some embodiments, the subject has previously received a PD-1 inhibitor, PD-L1 inhibitor, CTLA-4 inhibitor or any combination thereof.

In some embodiments, the subject has disease progression.

In some embodiments, the subject has received or is currently receiving a PD-1 inhibitor or PD-L1 inhibitor for at least 3 months.

In some embodiments, the subject has stable disease or asymptomatic progressive disease.

In some embodiments, the method further comprises depleting CD14+ cells from the biological sample prior to (a).

In some embodiments, the method further comprises depleting CD25+ cells from the biological sample prior to (a).

In some embodiments, the method further comprises depleting CD56+ cells from the biological sample prior to (a).

In some embodiments, the biological sample is peripheral blood mononuclear cell (PBMC) sample.

In some embodiments, the biological sample is a washed and/or cryopreserved peripheral blood mononuclear cell (PBMC) sample.

In some embodiments, the expanded population of T cells or the third population of T cells comprises from 1×10\hat{ }7 to 1×10\hat{ }11 total cells.

In some embodiments, the APCs comprise a polynucleotide encoding the epitope of the peptide antigen.

In some embodiments, the polynucleotide is mRNA.

In some embodiments, the APCs have been contacted with a polypeptide comprising the peptide antigen.

In some embodiments, the peptide antigen is a RAS peptide antigen.

In some embodiments, the method comprises selecting the epitope by a method comprising:
generating cancer cell nucleic acids from a first biological sample comprising cancer cells obtained from the subject and generating non-cancer cell nucleic acids from a second biological sample comprising non-cancer cells obtained from the same subject;
sequencing the cancer cell nucleic acids by whole genome sequencing or whole exome sequencing, thereby obtaining a first plurality of nucleic acid sequences comprising cancer cell nucleic acid sequences; and
sequencing the non-cancer cell nucleic acids by whole genome sequencing or whole exome sequencing, thereby obtaining a second plurality of nucleic acid sequences comprising non-cancer cell nucleic acid sequences;
identifying cancer specific nucleic acid sequences from the first plurality of nucleic acid sequences that (i) encode epitopes containing a cancer-specific mutation, (ii) that are specific to the cancer cells and (iii) that do not include a nucleic acid sequence from the second plurality of nucleic acid sequences;
predicting or calculating or measuring which epitopes form a complex with a protein encoded by an HLA allele of the same subject by an HLA peptide binding analysis; and
selecting an epitope predicted or calculated or measured in (d), to bind to the protein encoded by an HLA allele of the same subject with an IC50 of less than 500 nM.

In some embodiments, culturing a first population of T cells comprises adding a pulse amount of the peptide antigen prior to expanding the second population of T cells, prior to enriching CD137 (4-1BB)-expressing T cells.

In some embodiments, the pulse amount of the peptide is added at most about 2 days prior to expanding the second population of T cells, prior to enriching CD137 (4-1BB)-expressing T cells.

In some embodiments, the pulse amount of the peptide is higher than the first amount of the peptide antigen.

In some embodiments, the RAS peptide antigen is a RAS peptide neoantigen or an antigen derived from a RAS mutation.

A method for producing a therapeutic population of T cells comprising:
culturing a first population of T cells from a biological sample from a subject in a first cell culture medium comprising antigen presenting cells (APCs) to produce a second population of T cells, wherein the APCs present an epitope of a peptide antigen in complex with an MHC protein;
expanding the second population of T cells in a second cell culture medium comprising a first amount of the peptide antigen to produce a third population of T cells;
supplementing the second cell culture medium with a second amount of the peptide antigen, wherein the second amount of the peptide antigen is higher than the first amount of the peptide antigen; and
expanding the third population of T cells to obtain a therapeutic population of T cells comprising antigen-specific T cells.

A method for producing a therapeutic population of T cells comprising:
culturing a first population of T cells from biological sample from a subject in a first cell culture medium comprising antigen presenting cells (APCs) to produce a second population of T cells, wherein the APCs present an epitope of a peptide antigen in complex with an MHC protein;
enriching CD137 (4-1BB)-expressing T cells and/or CD69 expressing T cells to produce an enriched second population of T cells;
culturing the enriched population of T cells in a second culture medium that is supplemented with pulses of an increasing concentration of the peptide antigen, starting with a dose that is lower than that present in the first culture medium; and
expanding the enriched second population of T cells in a second cell culture medium to obtain a therapeutic population of T cells comprising antigen-specific T cells.

Also provided herein is pharmaceutical composition comprising a therapeutic population of T cells comprising antigen-specific T cells produced according to a method described herein.

Provided herein are methods to produce T cells for use as a therapeutic that use an initial ex vivo induction of naïve T cell responses or expansion of memory T cell responses followed by a peptide pulse to upregulate activation markers preferentially on antigen-specific T cells. Also provided herein are methods to produce T cells for use as a therapeutic that use an experimentally-defined expansion protocol that is optimized based on the specific enrichment marker used. Also provided herein are methods to produce T cells for use as a therapeutic that exponentially increasing peptide pulse during the expansion phase to preferentially re-stimulate antigen-specific T cells. The methods provided herein can involve using a stimulation protocol to initially induce and expand antigen-specific cells against particular antigens, allowing for the enrichment of de novo T cell responses. It was then determined that cells induced and expanded this way are able to upregulate cell-surface activation markers with a simple reintroduction of the inducing peptides rather than using an antigen presenting cell. When stimulated this way, the activation markers specifically and transiently increase on the antigen-specific cells, allowing them to be enriched using bead-based enrichment. Many methods for T cell expansion exist that focus on stimulation using cytokines (e.g., IL-2) or beads which activate co-stimulatory molecules on T cells (e.g., CD3/CD28). These methods are used to expand all cells in the culture, regardless of antigen-specificity. Since antigen-specific cells are generally of low frequency, a method to preferentially expand antigen-specific cells is needed. Expansion of solely antigen-specific cells via exponentially increasing doses of peptide is a novel concept that has not been widely studied. Indeed, this strategy has been studied in the context of T cell priming and in vaccinations. These strategies focus on early stages of T cell development.

Antigen-specific expansion of T cells via exponential peptide dosing is a process in which after initial culture, PBMCs are dosed with increasing amounts of the peptide(s) over the course of, for example, three days. For example, if PBMCs were primed against a KRAS G12 epitope, expansion would consist of pulsing the enriched PBMCs with exponentially increasing doses of the same KRAS G12 epitope. This strategy mimics that natural course of a viral infection in vivo and can be done for one of more immunogens at once. To apply this process, an enriched stimulated product can be pulsed with pre-manufactured immunogens that are specific for the patient's HLA and tumor mutation. The immunogen could be either a Class I or Class II epitope. The process of enriching and expanding antigen-specific T cells has been found to greatly increase the number of antigen-specific cells that make up the final cell therapy product and could be made up of both CD8 and CD4 T cells. This method has been conceived and validated using KRAS as a model neo-antigen but can apply to all induced T cell responses.

This disclosure provides novel and improved T cell therapeutics for clinical development and use. Although autologous T cell therapeutic is safe to use, several drastic improvements are necessary to meet therapeutic standards and development in the field has been both rapid and fraught with difficulties. Applicant's previously disclosed applications provide hallmark developments in the composition and methods for T cell therapy in cancer, (see WO2019/094642 and PCT/US2020/031898, each of which are incorporated by reference in its entirety). The instant application results from a surprising discovery that enrichment of certain cells expressing specific markers at different stages of the ex vivo immune cell preparation provides highly immunogenic cell compositions. The present disclosure is derived also in part from the discovery of new and improved methods for antigenic stimulation thereby resulting in improved cell composition for the therapeutics development. Provided herein are new methods and compositions wherein, at least in part, increasing amounts of peptide added during ex vivo stimulation and cell expansion provides new therapeutic compositions and improved methods.

Provided herein is a method for expanding T cells from a subject into a therapeutic population of antigen-specific T cells comprising: (a) culturing a first population of T cells from biological sample from a subject in a first cell culture medium comprising antigen presenting cells (APCs) to produce a second population of T cells, wherein the APCs present an epitope of a peptide antigen in complex with an MHC protein; (b) expanding the second population of T cells in a second cell culture medium comprising a first amount of the peptide antigen to produce a third population of T cells; (c) supplementing the second cell culture medium with a second amount of the peptide antigen, wherein the second amount of the peptide antigen is higher than the first amount of the peptide antigen; and (d) expanding the third population of T cells to obtain an expanded population of T cells.

Also provided herein is a method for expanding T cells from a subject into a therapeutic population of antigen-specific T cells comprising: (a) culturing a first population of T cells from biological sample from a subject in a first cell culture medium comprising antigen presenting cells (APCs) to produce a second population of T cells, wherein the APCs present an epitope of a peptide antigen in complex with an MHC protein; (b) enriching CD137 (4-1BB)-expressing T cells to produce an enriched second population of T cells; and (c) expanding the enriched second population of T cells in a second cell culture medium to obtain a third population of T cells.

Also provided herein is a method for expanding T cells from a subject into a therapeutic population of antigen-specific T cells comprising: (a) culturing a first population of T cells from biological sample from a subject in a first cell culture medium comprising antigen presenting cells (APCs) to produce a second population of T cells, wherein the APCs present an epitope of a peptide antigen in complex with an MHC protein; (b) enriching CD69-expressing T cells to produce an enriched second population of T cells; and (c) expanding the enriched second population of T cells in a second cell culture medium to obtain a third population of T cells.

In some embodiments, the method further comprises supplementing the second cell culture medium with a second amount of the peptide antigen, wherein the second amount of the peptide antigen is higher than the first amount of the peptide antigen.

In some embodiments, the method further comprises expanding the third population of T cells for a third period of from 1 to 20 days to obtain an expanded population of T cells.

In some embodiments, culturing of the first population of T cells is performed for first period of from 5 to 25 days, from 7 to 16 days, from 13 to 15 days or about 14 days to obtain the second population of T cells.

In some embodiments, the number of antigen-specific T cells in the second population of T cells is greater in number than the number of antigen-specific T cells in the first population of T cells.

In some embodiments, culturing of the enriched second population of T cells is performed for a second period of from 5 to 25 days, from 7 to 14 days, from 11 to 13 days, 21 days or less, or about 12 days to obtain the third population of T cells.

In some embodiments, expanding the third population of T cells comprises expanding the third population of T cells for a third period of from 4 to 24 days, from 6 to 13 days, from 10 to 12 days, 20 days or less, or about 11 days to obtain an expanded population of T cells.

In some embodiments, the method preferentially or specifically expands antigen-specific T cells.

In some embodiments, the method preferentially or specifically expands naive T cells from the first population of T cells.

In some embodiments, the method preferentially or specifically expands naive T cells from the first population of T cells that have become antigen-specific T cells.

In some embodiments, the third population of T cells or the expanded population of T cells is a therapeutic population of antigen-specific T cells.

In some embodiments, expanding the second population of T cells, expanding the enriched second population of T cells or expanding third population of T cells comprises expanding antigen-specific T cells.

In some embodiments, culturing the first population of T cells comprises expanding antigen-specific T cells.

In some embodiments, expanding the second population of T cells comprises expanding antigen-specific T cells.

In some embodiments, the first population of T cells is not obtained from a tumor infiltrating lymphocyte (TIL) sample.

In some embodiments, the first and second culture mediums are the same.

In some embodiments, the first and second culture mediums are different.

In some embodiments, the first culture medium comprises GM-CSF, IL-4, FLT3L, TNF-α, IL-1β, PGE1, IL-6, IL-7, IFN-α, R848, LPS, ss-rna40, poly I:C, or any combination thereof.

In some embodiments, the second culture medium comprises a soluble anti-CD3 antibody, an anti-CD3 antibody conjugated to a bead, soluble anti-CD28 antibody, an anti-CD28 antibody conjugated to a bead, insulin, one or more non-essential amino acids, glucose, glutamine, IL-2, IL-7, IL-15, IL-12, a CD137 agonist, an AKT inhibitor, a MEM vitamin solution, sodium pyruvate or any combination thereof.

In some embodiments, the first culture medium comprises FMS-like tyrosine kinase 3 receptor ligand (FLT3L).

In some embodiments, the second culture medium comprises FLT3L.

In some embodiments, the second culture medium does not comprise additional APCs.

In some embodiments, a number of APCs added in the supplementing step is less than the number of APCs present in the first cell culture medium.

In some embodiments, supplementing does not comprise supplementing with APCs.

In some embodiments, the method further comprises enriching CD137-expressing T cells or CD69-expressing T cells from the second population of T cells after (a) and before (b).

In some embodiments, enriching comprises enriching with an enriching reagent comprising an anti-CD69 reagent or an ant-CD137 reagent.

In some embodiments, the enriching reagent is an antibody or binding fragment thereof.

In some embodiments, the enriching reagent is coupled to a solid surface.

In some embodiments, enriching comprises immunoprecipitating.

In some embodiments, the second amount of the peptide antigen is at least 1.1, 1.5, 2, 3, 4, 5, 6, 7, 8, 9 or 10 times higher than the first amount of the peptide antigen.

In some embodiments, the method further comprises supplementing the second cell culture medium with a third amount of the peptide antigen, wherein the third amount of the peptide antigen is higher than the second amount of the peptide antigen.

In some embodiments, the third amount of the peptide antigen is at least 1.1, 1.5, 2, 3, 4, 5, 6, 7, 8, 9 or 10 times higher than the second amount of the peptide antigen.

In some embodiments, the method further comprises harvesting the expanded population of T cells or the third population of T cells.

In some embodiments, the method further comprises transferring the harvested population of T cells to an infusion bag.

In some embodiments, the method further comprises administering the expanded population of T cells or the third population of T cells to the subject.

In some embodiments, the subject has a disease or condition.

In some embodiments, the disease or condition is cancer.

In some embodiments, the cancer is a solid cancer.

In some embodiments, the cancer is melanoma, pancreatic ductal adenocarcinoma (PDAC), colorectal cancer (CRC) or non-small cell lung cancer (NSCLC).

In some embodiments, the cancer is unresectable melanoma or RAS mutant PDAC.

In some embodiments, the subject is human.

In some embodiments, the subject has previously received a PD-1 inhibitor, PD-L1 inhibitor, CTLA-4 inhibitor or any combination thereof.

In some embodiments, the subject has disease progression.

In some embodiments, the subject has received or is currently receiving a PD-1 inhibitor or PD-L1 inhibitor for at least 3 months.

In some embodiments, the subject has stable disease or asymptomatic progressive disease.

In some embodiments, the method further comprises depleting CD14+ cells from the biological sample prior to (a).

In some embodiments, the method further comprises depleting CD25+ cells from the biological sample prior to (a).

In some embodiments, the biological sample is peripheral blood mononuclear cell (PBMC) sample.

In some embodiments, the biological sample is a washed and/or cryopreserved peripheral blood mononuclear cell (PBMC) sample.

In some embodiments, the expanded population of T cells or the third population of T cells comprises from 1×10⁷ to 1×10¹¹ total cells.

In some embodiments, the APCs comprise a polynucleotide encoding the epitope of the peptide antigen.

In some embodiments, the polynucleotide is mRNA.

In some embodiments, the APCs have been contacted with a polypeptide comprising the peptide antigen.

In some embodiments, the peptide antigen is a RAS peptide antigen, such as a RAS peptide antigen of any one of Tables 1-14.

In some embodiments, the RAS peptide antigen is a RAS peptide neoantigen or an antigen derived from a RAS mutation.

In some embodiments, the method comprises selecting the epitope by a method comprising: (a) generating cancer cell nucleic acids from a first biological sample comprising cancer cells obtained from the subject and generating non-cancer cell nucleic acids from a second biological sample comprising non-cancer cells obtained from the same subject; (b) sequencing the cancer cell nucleic acids by whole genome sequencing or whole exome sequencing, thereby obtaining a first plurality of nucleic acid sequences comprising cancer cell nucleic acid sequences; and sequencing the non-cancer cell nucleic acids by whole genome sequencing or whole exome sequencing, thereby obtaining a second plurality of nucleic acid sequences comprising non-cancer cell nucleic acid sequences; (c) identifying cancer specific nucleic acid sequences from the first plurality of nucleic acid sequences that (i) encode epitopes containing a cancer-specific mutation, (ii) that are specific to the cancer cells and (iii) that do not include a nucleic acid sequence from the second plurality of nucleic acid sequences; (d) predicting or calculating or measuring which epitopes form a complex with a protein encoded by an HLA allele of the same subject by an HLA peptide binding analysis; and (e) selecting an epitope predicted or calculated or measured in (d), to bind to the protein encoded by an HLA allele of the same subject with an IC50 of less than 500 nM.

In some embodiments, culturing a first population of T cells comprises adding a pulse amount of the peptide antigen prior to expanding the second population of T cells, prior to enriching CD137 (4-1BB)-expressing T cells, or prior to enriching CD69-expressing T cells.

In some embodiments, the pulse amount of the peptide is added at most about 2 days prior to expanding the second population of T cells, prior to enriching CD137 (4-1BB)-expressing T cells, or prior to enriching CD69-expressing T cells.

In some embodiments, the pulse amount of the peptide is higher than the first amount of the peptide antigen.

Also provided herein is a pharmaceutical composition comprising a therapeutic population of antigen-specific T cells produced according to a method described herein.

### BRIEF DESCRIPTION OF THE DRAWINGS

FIG. 1A depicts an exemplary schematic of an ex vivo manufacturing protocol to produce T cells specific to a complex comprising a peptide (KRAS) and an MHC protein. This protocol can be used to produce T cells specific to any peptide:MHC complexes, such as patient-specific peptide:MHC complexes. The basic protocol steps with an exemplary timeline starting from Day 0 to completion and harvesting at Day 26 are depicted.

FIG. 1B depicts an exemplary schedule and processing steps for the exemplary manufacturing protocol depicted in FIG. 1A to obtain a therapeutic population of antigen specific T cells.

FIG. 2 depicts an exemplary schedule and processing steps for the manufacturing protocol to obtain a therapeutic population of antigen specific T cells.

FIG. 3 depicts an exemplary schematic of an experiment to evaluate parameters that affect the enrichment step of the ex vivo manufacturing protocol shown in FIG. 1A. A timeline is depicted on the top section of the figure. At Day 14, the expanding cell population is enriched using an anti-CD137 antibody, thereby increasing the proportion of antigen specific T cells in the enriched cell population compared to the cell population prior to enrichment. The enriched cell population is further until being harvested and analyzed by flow cytometry at Day 26.

FIG. 4 depicts data showing enrichment of T cells is feasible in a large scale manufacturing process. In both tables, attributes of the cells after two different CliniMACS runs are shown, including viability, yield of the 4-1BB fraction relative to the input, purity of 4-1BB, multimer frequency after enrichment, and recovery of antigen-specific cells relative to input. The left table shows a comparison between Method 2.1 and Method 3.2 on the CliniMACS machine. The right table shows a comparison between MACS buffer and AIM-V as the running buffer.

FIG. 5 depicts exemplary flow cytometry data demonstrating expression of the indicated activation markers on T cell populations stimulated overnight with antigenic peptides (bottom panel) or without antigenic peptides (control, upper panel). The y-axis of each plot is the peptide-MHC multimer conjugated to a fluorophore and the x-axis is the indicated activation marker. Multimer negative (grey) and multimer positive (dark) cell populations are shown.

FIG. 6 depicts exemplary data showing activation marker expression over time in hours following peptide stimulation as indicated in the figure.

FIG. 7 depicts exemplary results across multiple experiments showing the percent of CD8+ T cells specific for KRAS peptide:MHC complexes after CD137 enrichment (left) and CD69 enrichment (right), over no enrichment, measured as a percent of total CD8+ T cells. The lower panel provides average fold enrichment from a separate experiment with either CD137 selection marker or CD69 selection marker. Antigen-specific responses that could be detected without enrichment were defined as pre-existing or non-novel, whereas those that were detected only with enrichment were defined as novel responses.

FIG. 8A depicts exemplary results multimer frequency of CD8+ T cells specific for three highly immunogenic epitopes after CD137 enrichment. Increase in multimer positive CD8 T cells as a percent of total CD8+ T cells is demonstrated in presence of enrichment using CD137 positive selection, compared to no enrichment.

FIG. 8B depicts exemplary flow cytometry results demonstrating an increase in antigen specific CD8+ T cells in a CD137 enriched population (right graph) compared to no enrichment (left graph).

FIG. 9 depicts exemplary cytometry data showing multimer positive and multimer negative cells without enrichment, with CD137 (4-1BB) enrichment or with CD69 enrichment. The graph on the lower left depicts exemplary enrichment data using CD137 enrichment (light squares) or CD69 enrichment (dark circles) plotted against data prior to enrichment. Enriched cells are represented as antigen specific CD8+ T cells as a percent of total CD8+ T cells. The lower right panel fold change of the antigen-specific frequency for either CD137 or CD69 enrichment protocols relative to the pre-enrichment frequency.

FIG. 10A depicts exemplary results of the multimer positive frequency of CD8+ cells (%) after enrichment using varying concentrations of CD137 and/or CD69 capture antibody beads as indicated on the X-axis. Decreased concentrations of the antibodies, depicted by the fold dilution (e.g. 1×, indicating no dilution, 1/5×indicating 5-fold dilution and 1/25×indicating 25-fold dilution), increases antigen-specific frequency post-enrichment.

FIG. 10B depicts exemplary results of the percent multimer positive CD8+ cells recovered following antibody-based isolation using MACS separation protocols.

FIG. 11 depicts exemplary data indicating increasing dilution of the CD137 (4-1BB) antibody results in higher antigen-specific frequency post-enrichment.

FIG. 12 depicts an exemplary study design for expansion of T cells to investigate the effects of the indicated T cell activator reagents used during the expansion process. After enrichment, the cells are divided into three groups: treatment with (1) no activator control, (2) treatment with CD3/CD28 beads (beads:cell 1:1) or (3) treatment with soluble CD3/CD28 for activator (25 μL/10 \hat{ }6 cells) during expansion phase. Expansion was done in basal media containing AIM-V buffer, in presence of IL-7, IL-15, IL-2 and 5% Human Serum.

FIG. 13 depicts exemplary data showing the percentage of RAS-specific T cells of total CD8+ T cells before enrichment and after CD137 or CD69 enrichment and expansion in the presence of IL-7, IL-15 and IL-2 and in the presence or absence of CD3/CD28 beads or CD3/CD28 (soluble). Three different healthy donors were evaluated (circle, square and triangle symbols).

FIG. 14A depicts exemplary data following expansion of KRAS antigen specific CD8+ T cells following enrichment of CD69 expressing cells (1) in the presence or absence of soluble CD3/CD28 T cell activator reagent and (2) in the presence of a constant amount of peptide antigen, no peptide antigen and increasing amounts of peptide antigen added over time during expansion. The data shows an unexpected finding that antigen specific cell expansion is strongly affected by exponential peptide pulsing on days 15-17 after enrichment (CD69). The graph on the left shows that with exponential peptide pulsing total antigen-specific T cell numbers increase by greater than 4-fold.

FIG. 14B shows exemplary data indicating that the percentage of multimer positive cells of total CD8+ cells increased when increasing amounts of peptide antigen was added over time during expansion (average=>4×) compared to no peptide.

FIG. 15 shows exemplary data of the mean (absolute combined) multimer positive cells after expansion of cells in the absence or presence of a high or low amount of a soluble CD3/CD28 activator with or without increasing amounts of peptide antigen added over time during expansion. These results indicate that the exponential peptide pulse was most effective in absence of the activator, and that exponential peptide pulse is effective with CD137 enrichment.

FIG. 16 depicts exemplary data showing implementation of enrichment/expansion step reliably increases antigen-specific frequency in final product. The percentage of antigen-specific T cells before enrichment, after enrichment, and after expansion are shown for 4 different runs. The enrichment is performed as depicted in FIG. 3 and FIG. 4 and the expansion is performed as shown in FIG. 12 with the addition of exponential peptide pulsing. These results indicate the antigen-specific frequency is increased both by the enrichment process and expansion process.

FIG. 17A shows a representative data showing expansion of KRAS antigen specific T cells on day 26 (after expansion) relative to Day 14 (before expansion). Data shows that the cumulative KRAS antigen-specific T cells of total CD8+ T cells increases by 47-fold during the expansion process as depicted in FIG. 12 with the addition of exponential peptide pulsing and anti-CD28. Three individual KRAS specificities are shown.

FIG. 17B shows another representative data showing expansion of KRAS antigen specific T cells at enrichment step (left graph) and at the end of expansion (right graph).

FIG. 18 depicts exemplary data showing that the KRAS antigen-specific T cells maintains cytotoxic capacity after expansion. Data shows that the KRAS antigen-specific T cells kill GFP positive target cells expressing the target mutations, but not loaded with wild-type peptides where GFP positive target cell numbers continue to increase until saturation.

FIG. 19 depicts data indicating enrichment of CD137+ T cells or CD69+ T cells increases antigen-specific CD4+ T cell fraction. Left graph demonstrates that peptide spike increases activation and functional markers on CD4+ T cells (IFN-γ, TNFα, 4-1BB, CD69) with (left) or without (right) added APCs. Right graph shows increase in antigen specific CD4+ T cell fraction in cells expanded from PBMC of three different donors. The cells were expanded with another non-KRAS epitope. Square solid datapoints indicate enrichment of CD137+ cells.

FIG. 20 depicts an exemplary data showing increasing (exponential) peptide pulse during expansion leads to preferential growth of KRAS-specific T cells.

FIG. 21 depicts an exemplary data showing increasing (exponential) peptide pulse preferentially expands the strongest avidity TCRs based on lowest EC50 in a peptide titration assay.

FIG. 22 depicts data illustrating antigen specificity of the T cells, indicated by target cell specific cytotoxicity of the T cells. Graphs show % cell death of the specific RAS mutation expressing cells, and not WT RAS expressing cells.

FIG. 23 depicts data showing that the expansion protocol alone can increase antigen specific T cells independent of the enrichment step.

FIG. 24 depicts data indicating successful scalability and large scale production of the mKRAS specific T cells using the disclosed protocol.

FIG. 25A depicts an observation of a high frequency of NK cells in the large scale products.

FIG. 25B depicts data indicating that further depletion of CD56+ cells in addition to CD14+ and CD25+ cell depletion as indicated in the protocols depicted above at day 0 removes CD56+(NK cells) but also CD3+CD56+ cells (left graph). At day 26, following enrichment and expansion, CD56 depletion led to lower NK cell frequencies and an increased CD3+CD56− cells (upper right), with increased antigen specific cell numbers (lower right).

### DETAILED DESCRIPTION

A T cell therapeutic is expected to be a relatively safe and well-tolerated adoptive T cell product. However, based on an assessment of the risks associated with the product, there are 3 general classes of potential toxicities associated with a T cell therapeutic: (a) treatment related toxicity due to lymphodepletion, cell infusion, or cytokine release syndrome; (b) off-tumor, off-target toxicity due to the expansion of autoreactive clones or cross reactivity of the neoantigen specific T cells; and (c) off-tumor, on-target toxicity due to the presentation of the neoantigens on non-tumor tissue. Described herein are novel immunotherapeutic agents and uses thereof based on the discovery of neoantigens arising from mutational events unique to an individual's tumor. Accordingly, the present disclosure described herein provides methods and protocols to create antigen specific immune cells, for example T cells, for use in treating disease.

Presented herein is a composition of neoantigen responsive T cells for cancer immunotherapy. Although adoptive T cell therapy is a promising new approach for cancer therapy it requires several improvements. Generally, the T cells have to be adequately cytotoxic to cancer cells, have to spare the non-cancer cells in the body, should not lose immunogenicity in the tumor environment and should offer long term protection. Additionally, use of virally transduced cells has its own challenges. Therefore, striking the right balance to achieve therapeutically effective composition which specifically target cancer cells, sparing healthy cell, stall the progress of the disease, cause amelioration or at least substantial tumor regression and prevent relapse of the cancer, requires several improvements in almost all the steps of the complex process.

To facilitate an understanding of the present disclosure, a number of terms and phrases are defined below.

An antigen is a foreign substance to the body that induces an immune response. A “neoantigen” refers to a class of tumor antigens which arise from tumor-specific changes in proteins. Neoantigens encompass, but are not limited to, tumor antigens which arise from, for example, a substitution in a protein sequence, a frame shift mutation, a fusion polypeptide, an in-frame deletion, an insertion, and expression of an endogenous retroviral polypeptide.

A “neoepitope” refers to an epitope that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope.

A “mutation” refers to a change of or a difference in a nucleic acid sequence (e.g., a nucleotide substitution, addition or deletion) compared to a reference nucleic acid. A “somatic mutation” can occur in any of the cells of the body except the germ cells (sperm and egg) and are not passed on to children. These alterations can (but do not always) cause cancer or other diseases. In some embodiments, a mutation is a non-synonymous mutation. A “non-synonymous mutation” refers to a mutation, for (e.g., a nucleotide substitution), which does result in an amino acid change such as an amino acid substitution in the translation product. A “frameshift” occurs when a mutation disrupts the normal phase of a gene's codon periodicity (also known as “reading frame”), resulting in translation of a non-native protein sequence. It is possible for different mutations in a gene to achieve the same altered reading frame.

“Antigen processing” or “processing” refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

An “antigen presenting cell” (APC) refers to a cell which presents peptide fragments of protein antigens in association with MHC molecules on its cell surface. The term includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes).

The term “affinity” refers to a measure of the strength of binding between two members of a binding pair (e.g., a human leukocyte antigen (HLA)-binding peptide and a class I or II HLA, or a peptide-HLA complex and a T cell receptor (TCR)). K_{D} refers to the dissociation constant between two members of a binding pair and has units of molarity. K_{A} refers to the affinity constant between two members of a binding pair is the inverse of the dissociation constant. Affinity may be determined experimentally, for example by surface plasmon resonance (SPR) using commercially available Biacore SPR units. K_{off} refers to the off-rate constant of two members of a binding pair, (e.g., the off-rate constant of an HLA-binding peptide and a class I or II HLA, or a peptide-HLA complex and a TCR). Kₒₙ refers to the on-rate constant of two members of a binding pair, (e.g., the on-rate constant of an HLA-binding peptide and a class I or II HLA, or a peptide-HLA complex and a TCR).

Throughout this disclosure, “binding data” results may be expressed in terms of an “IC₅₀.” Affinity may also be expressed as the inhibitory concentration 50 (IC₅₀), or the concentration at which 50% of a first member of a binding pair (e.g., a peptide) is displaced. Likewise, ln(IC₅₀) refers to the natural log of the IC₅₀. For example, an IC₅₀ may be the concentration of a tested peptide in a binding assay at which 50% inhibition of binding of a labeled reference peptide is observed. Given the conditions in which the assays are run (e.g., limiting HLA protein concentrations and/or labeled reference peptide concentrations), these values can approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

The term “derived” when used to discuss an epitope is a synonym for “prepared.” A derived epitope can be isolated from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine A derived or prepared epitope can be an analog of a native epitope. The term “derived from” refers to the origin or source, and may include naturally occurring, recombinant, unpurified, purified or differentiated molecules or cells. For example, an expanded or induced antigen specific T cell may be derived from a T cell. For example, an expanded or induced antigen specific T cell may be derived from an antigen specific T cell in a biological sample. For example, a matured APC (e.g., a professional APC) may be derived from a non-matured APC (e.g., an immature APC). For example, an APC may be derived from a monocyte (e.g., a CD14⁺ monocyte). For example, a dendritic cell may be derived from a monocyte (e.g., a CD14⁺ monocyte). For example, an APC may be derived from a bone marrow cell.

An “epitope” is the collective features of a molecule (e.g., a peptide's charge and primary, secondary and tertiary structure) that together form a site recognized by another molecule (e.g., an immunoglobulin, T cell receptor, HLA molecule, or chimeric antigen receptor). For example, an epitope can be a set of amino acid residues involved in recognition by a particular immunoglobulin; a Major Histocompatibility Complex (MHC) receptor; or in the context of T cells, those residues recognized by a T cell receptor protein and/or a chimeric antigen receptor. Epitopes can be prepared by isolation from a natural source, or they can be synthesized according to standard protocols in the art. Synthetic epitopes can comprise artificial amino acid residues, amino acid mimetics, (such as D isomers of naturally-occurring L amino acid residues or non-naturally-occurring amino acid residues). Throughout this disclosure, epitopes may be referred to in some cases as peptides or peptide epitopes. In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of: less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues.

A “T cell epitope” refers to a peptide sequence bound by an MHC molecule in the form of a peptide-MHC (pMHC) complex. A peptide-MHC complex can be recognized and bound by a TCR of a T cell (e.g., a cytotoxic T-lymphocyte or a T-helper cell).

A “T cell” includes CD4⁺ T cells and CD8⁺ T cells and any T lymphocyte as is understood by one of skill in the art. T cells are characterized by expression of cell surface marker CD3. T cells are generally of three main types, cytotoxic, helper and regulatory. T cells that are responsible for immune activation and immune response are typically the cytotoxic T cell, also known as CD8+ T cells, and the helper T cells, also known as CD4+ T cells. Regulatory T cells have an immunoregulatory role, geared towards immune suppression. Functionally, T cells may be further subcategorized and variously termed, for example, as memory T cells, naïve T cells, or antigen primed T cells. The term T cell also includes both T helper 1 type T cells and T helper 2 type T cells. T cells may be generated by the method described in the application, for a clinical application. T cells or adoptive T cells referred to here, such as for a clinical application are cells isolated from a biological source, manipulated and cultured ex vivo and prepared into a drug candidate for a specific therapy such as a cancer, e.g., melanoma. When drug candidate cells pass specific qualitative and quantitative criteria for fitness for a clinical application, the drug candidate may be designated a drug product. In some cases, a drug product is selected from a number of drug candidates. In the context of this application, a drug product is a T cell, more specifically, a population of T cells, or more specifically a population of T cells with heterogeneous characteristics and subtypes. For example, a drug product, as disclosed herein may have a population of T cells comprising CD8+ T cells, CD4+ T cells, with cells at least above a certain exhibiting antigen specificity, a certain percentage of each exhibiting a memory phenotype, among others.

An “immune cell” refers to a cell that plays a role in the immune response Immune cells are of hematopoietic origin, and include lymphocytes, such as B cells and T cells; natural killer cells; myeloid cells, such as monocytes, macrophages, eosinophils, mast cells, basophils, and granulocytes.

An “immunogenic” peptide or an “immunogenic” epitope or an “immunogenic” peptide epitope is a peptide that binds to an HLA molecule and induces a cell-mediated or humoral response, for example, a cytotoxic T lymphocyte (CTL) response, a helper T lymphocyte (HTL) response and/or a B lymphocyte response Immunogenic peptides described herein are capable of binding to an HLA molecule and thereafter induce a cell-mediated or humoral response (e.g., a CTL (cytotoxic) response, or a HTL response) to the peptide.

A “protective immune response” or “therapeutic immune response” refers to a CTL and/or an HTL response to an antigen derived from an pathogenic antigen (e.g., a tumor antigen), which in some way prevents or at least partially arrests disease symptoms, side effects or progression. The immune response can also include an antibody response which has been facilitated by the stimulation of helper T cells.

A “T cell receptor” (“TCR”) refers to a molecule, whether natural or partly or wholly synthetically produced, found on the surface of T lymphocytes (T cells) that recognizes an antigen bound to a major histocompatibility complex (MHC) molecule. The ability of a T cells to recognize an antigen associated with various diseases (e.g., cancers) or infectious organisms is conferred by its TCR, which is made up of both an alpha (α) chain and a beta (β) chain or a gamma (γ) and a delta (δ) chain. The proteins which make up these chains are encoded by DNA, which employs a unique mechanism for generating the tremendous diversity of the TCR. This multi-subunit immune recognition receptor associates with the CD3 complex and binds peptides presented by the MHC class I and II proteins on the surface of antigen-presenting cells (APCs). Binding of a TCR to a peptide on an APC is a central event in T cell activation.

As used herein, a “chimeric antigen receptor” or “CAR” refers to an antigen binding protein in that includes an immunoglobulin antigen binding domain (e.g., an immunoglobulin variable domain) and a T cell receptor (TCR) constant domain. As used herein, a “constant domain” of a TCR polypeptide includes a membrane-proximal TCR constant domain, a TCR transmembrane domain and/or a TCR cytoplasmic domain, or fragments thereof. For example, in some embodiments, a CAR is a monomer that includes a polypeptide comprising an immunoglobulin heavy chain variable domain linked to a TCRβ constant domain. In some embodiments, the CAR is a dimer that includes a first polypeptide comprising an immunoglobulin heavy or light chain variable domain linked to a TCRα or TCRβconstant domain and a second polypeptide comprising an immunoglobulin heavy or light chain variable domain (e.g., a κ or λ variable domain) linked to a TCRβ or TCRα constant domain.

“Major Histocompatibility Complex” or “MHC” is a cluster of genes that plays a role in control of the cellular interactions responsible for physiologic immune responses. The terms “major histocompatibility complex” and the abbreviation “MHC” can include any class of MHC molecule, such as MHC class I and MHC class II molecules, and relate to a complex of genes which occurs in all vertebrates. In humans, the MHC complex is also known as the human leukocyte antigen (HLA) complex. Thus, a “Human Leukocyte Antigen” or “HLA” refers to a human Major Histocompatibility Complex (MHC) protein (see, e.g., Stites, et al., Immunology, 8^{TH} Ed., Lange Publishing, Los Altos, Calif (1994). For a detailed description of the MHC and HLA complexes, see, Paul, Fundamental Immunology, 3^{rd} Ed., Raven Press, New York (1993).

The major histocompatibility complex in the genome comprises the genetic region whose gene products expressed on the cell surface are important for binding and presenting endogenous and/or foreign antigens and thus for regulating immunological processes. MHC proteins or molecules are important for signaling between lymphocytes and antigen presenting cells or diseased cells in immune reactions. MHC proteins or molecules bind peptides and present them for recognition by T-cell receptors. The proteins encoded by the MHC can be expressed on the surface of cells, and display both self-antigens (peptide fragments from the cell itself) and non-self-antigens (e.g., fragments of invading microorganisms) to a T-cell. MHC binding peptides can result from the proteolytic cleavage of protein antigens and represent potential lymphocyte epitopes. (e.g., T cell epitope and B cell epitope). MHCs can transport the peptides to the cell surface and present them there to specific cells, such as cytotoxic T-lymphocytes, T-helper cells, or B cells. The MHC region can be divided into three subgroups, class I, class II, and class III. MHC class I proteins can contain an α-chain and β2-microglobulin (not part of the MHC encoded by chromosome 15). They can present antigen fragments to cytotoxic T-cells. MHC class II proteins can contain α- and β-chains and they can present antigen fragments to T-helper cells. MHC class III region can encode for other immune components, such as complement components and cytokines. The MHC can be both polygenic (there are several MHC class I and MHC class II genes) and polymorphic (there are multiple alleles of each gene).

A “receptor” refers to a biological molecule or a molecule grouping capable of binding a ligand. A receptor may serve, to transmit information in a cell, a cell formation or an organism. A receptor comprises at least one receptor unit, for example, where each receptor unit may consist of a protein molecule. A receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length. A “ligand” refers to a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with MHC proteins such as MHC class I or MHC class II proteins. In some embodiments, a “receptor/ligand complex” is also to be understood as meaning a “receptor/peptide complex” or “receptor/peptide fragment complex”, including a peptide- or peptide fragment-presenting MHC molecule such as MHC class I or MHC class II molecules.

A “native” or a “wild type” sequence refers to a sequence found in nature. The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

The terms “peptide” and “peptide epitope” are used interchangeably with “oligopeptide” in the present specification to designate a series of residues connected one to the other, typically by peptide bonds between the α-amino and carboxyl groups of adjacent amino acid residues. A “synthetic peptide” refers to a peptide that is obtained from a non-natural source, e.g., is man-made. Such peptides can be produced using such methods as chemical synthesis or recombinant DNA technology. “Synthetic peptides” include “fusion proteins.”

The term “motif” refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 7, 8 9, 10, 11, 12 or 13 amino acid residues in length. In some embodiments, an MHC class II motif identifies a peptide of 15, 16, 17, 18, 19, 20, 21, 22, 23, 24, 25 or 26 amino acid residues in length. A “cross-reactive binding” peptide refers to a peptide that binds to more than one member of a class of a binding pair members (e.g., a peptide bound by both a class I HLA molecule and a class II HLA molecule).

The term “residue” refers to an amino acid residue or amino acid mimetic residue incorporated into a peptide or protein by an amide bond or amide bond mimetic, or that is encoded by a nucleic acid (DNA or RNA). The nomenclature used to describe peptides or proteins follows the conventional practice. The amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with the first position being the residue located at the amino terminal end of the epitope, or the peptide or protein of which it can be a part. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.)

A “conservative amino acid substitution” is one in which one amino acid residue is replaced with another amino acid residue having a similar side chain Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

“Pharmaceutically acceptable” refers to a generally non-toxic, inert, and/or physiologically compatible composition or component of a composition. A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

According to the present disclosure, the term “vaccine” relates to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” “personal cancer vaccine” concerns a particular cancer patient and means that a cancer vaccine is adapted to the needs or special circumstances of an individual cancer patient.

The terms “polynucleotide” and “nucleic acid” are used interchangeably herein and refer to polymers of nucleotides of any length, and include DNA and RNA, for example, mRNA. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the invention is mRNA.

The terms “isolated” or “biologically pure” refer to material which is substantially or essentially free from components which normally accompany the material as it is found in its native state. Thus, isolated peptides described herein do not contain some or all of the materials normally associated with the peptides in their in situ environment. For example, an “isolated” epitope can be an epitope that does not include the whole sequence of the protein from which the epitope was derived. For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically. In some embodiments, a polypeptide, antibody, polynucleotide, vector, cell, or composition which is isolated is substantially pure. The term “substantially pure” as used herein refers to material which is at least 50% pure (i.e., free from contaminants), at least 90% pure, at least 95% pure, at least 98% pure, or at least 99% pure.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as an amino acid sequence of a peptide or a coding region of a nucleotide sequence.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus, those in need of treatment include those already with the disorder; those prone to have the disorder; and those in whom the disorder is to be prevented.

The term “depleted” when used to describe a cell sample (e.g., a peripheral blood mononuclear cell (PBMC) sample) refers to a cell sample in which a subpopulation of cells has been removed or depleted. For example, an immune cell sample depleted of CD25 expressing cells refers to an immune cell sample in which CD25 expressing cells have been removed or depleted. For example, one or more binding agents can be used to remove or deplete one or more cells or cell types from a sample. For example, CD14⁺ cells can be depleted or removed from a PBMC sample, such as by using an antibody that binds to CD14.

The “stimulation” refers to a response induced by binding of a stimulatory molecule with its cognate ligand thereby mediating a signal transduction event. For example, stimulation of a T cell can refer to binding of a TCR of a T cell to a peptide-MHC complex. For example, stimulation of a T cell can refer to a step within protocol 1 or protocol 2 in which PBMCs are cultured together with peptide loaded APCs.

The term “enriched” refers to a composition or fraction wherein an object species has been partially purified such that the concentration of the object species is substantially higher than the naturally occurring level of the species in a finished product without enrichment. The term “induced cell” refers to a cell that has been treated with an inducing compound, cell, or population of cells that affects the cell's protein expression, gene expression, differentiation status, shape, morphology, viability, and the like.

A “reference” can be used to correlate and/or compare the results obtained in the methods of the present disclosure from a diseased specimen. Typically, a “reference” may be obtained on the basis of one or more normal specimens, in particular specimens which are not affected by a disease, either obtained from an individual or one or more different individuals (e.g., healthy individuals), such as individuals of the same species. A “reference” can be determined empirically by testing a sufficiently large number of normal specimens.

As used herein, a tumor unless otherwise mentioned, is a cancerous tumor, and the terms cancer and tumor are used interchangeably throughout the document. While a tumor is a cancer of solid tissue, several of the compositions and methods described herein are in principle applicable to cancers of the blood, leukemia.

### Overview of T Cell Therapies

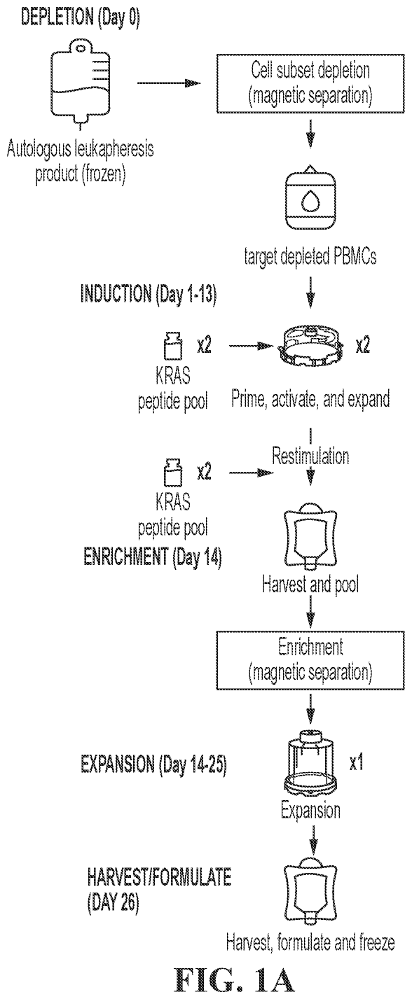

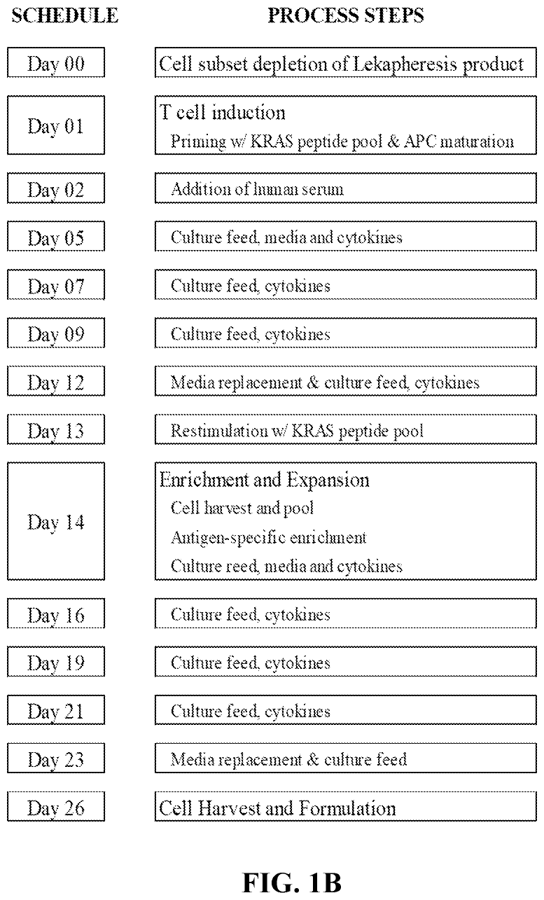

Generating antigen specific T cells by controlled ex vivo induction or expansion of T cells (e.g., autologous T cells) can provide highly specific and beneficial T cell therapies (e.g., adoptive T cell therapies). The present disclosure provides T cell manufacturing methods and therapeutic T cell compositions which can be used for treating subjects with cancer and other conditions, diseases and disorders. The objective is to expand and induce antigen specific T cells with a favorable phenotype and function. The present disclosure provides compositions and methods for manufacturing of T cells which can be used for antigen specific T cell therapy (e.g., personal or personalized T cell therapies). The T cell compositions provided herein can be personal antigen specific T cell therapies. FIG. 1 graphically represents an overview of the process related to T cell therapy: which includes on one hand, identification of the cancer and cancer specific antigens in the subject having the cancer, leading to the production of neoantigenic peptides; and on the other hand, preparing activated, antigen specific cells for immunotherapy and administering the cellular product.

### Neoantigen-Specific T Cell-Based Therapy

Traditional antigen-targeted immunotherapies have focused on tumor associated antigens (TAAs), antigens including cancer testes antigens (typically germ line restricted gene products which are aberrantly expressed in tumors) or antigens derived from genes which show tissue specific expression. However, tumors also display protein products of mutated genes which are called neoantigens. The number and type of mutations can be readily defined using next generation sequencing approaches and include single amino acid missense mutations, fusion protein, and novel open reading frames (neoORFs) varying in length from one up to one hundred or more amino acids. Neoantigens are antigens that comprise a non-silent mutation in an epitope, and the same antigen is not expressed in a non-cancer cell within the same human body. Mutation-based antigens are particularly valuable as these have bypassed central tolerance (the process which occurs during normal thymic development of removing self-reactive T cells) and demonstrate exquisite tumor specificity. Each nonsynonymous (i.e., protein coding) mutation has the potential to generate a neoantigen that can be recognized by the patient's T cells. T cells recognizing these neoantigens can function both to kill tumor cells directly and to catalyze a broader immune response against the tumor. The methods described herein aim to induce and expand such neoantigen-reactive T cells in a patient-specific fashion and utilize these cells for adoptive cell therapy.

In some embodiments, the neoantigens used herein comprises a point mutation.

In some embodiments, the neoantigens used herein comprises a frameshift mutation.

In some embodiments, the neoantigens used herein comprises a crossover mutation.

In some embodiments, the neoantigens used herein comprises an insertion mutation, caused by the insertion of one or more than one nucleotides.

In some embodiments, the neoantigens used herein comprises a deletion mutation, caused by the deletion of one or more than one nucleotides.

In some embodiments, the neoantigens may be caused by a insertion-deletion (in-del) mutation.

In some embodiments, an antigen or neoantigen peptide binds an HLA protein (e.g., HLA class I or HLA class II). In specific embodiments, an antigen or neoantigen peptide binds an HLA protein with greater affinity than a corresponding wild-type peptide. In specific embodiments, an antigen or neoantigen peptide has an IC₅₀ or K_{D} of at least less than 5000 nM, at least less than 500 nM, at least less than 100 nM, at least less than 50 nM or less.

In some embodiments, an antigen or neoantigen peptide can be from about 8 and about 50 amino acid residues in length, or from about 8 and about 30, from about 8 and about 20, from about 8 and about 18, from about 8 and about 15, or from about 8 and about 12 amino acid residues in length. In some embodiments, an antigen or neoantigen peptide can be from about 8 and about 500 amino acid residues in length, or from about 8 and about 450, from about 8 and about 400, from about 8 and about 350, from about 8 and about 300, from about 8 and about 250, from about 8 and about 200, from about 8 and about 150, from about 8 and about 100, from about 8 and about 50, or from about 8 and about 30 amino acid residues in length.

In some embodiments, an antigen or neoantigen peptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the neoantigen peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500 or more amino acid residues in length. In some embodiments, an antigen or neoantigen peptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, an antigen or neoantigen peptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

In some embodiments, an antigen or neoantigen peptide has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids.

In some embodiments, an antigen or neoantigen peptide has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids.

In some embodiments, the neoantigen peptides can have a pI value of about 0.5 and about 12, about 2 and about 10, or about 4 and about 8. In some embodiments, the neoantigen peptides can have a pI value of at least 4.5, 5, 5.5, 6, 6.5, 7, 7.5, or more. In some embodiments, the neoantigen peptides can have a pI value of at most 4.5, 5, 5.5, 6, 6.5, 7, 7.5, or less.

In some embodiments, an antigen or neoantigen peptide can have an HLA binding affinity of from about 1 pM and about 1 mM, about 100 pM and about 500 μM, about 500 pM and about 10 μM, about 1 nM and about 1 μM, or about 10 nM and about 1 μM. In some embodiments, an antigen or neoantigen peptide can have an HLA binding affinity of at least 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900 μM, or more. In some embodiments, an antigen or neoantigen peptide can have an HLA binding affinity of at most 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900 μM.

In some embodiments, an antigen or neoantigen peptide described herein can comprise carriers such as those well known in the art, e.g., thyroglobulin, albumins such as human serum albumin, tetanus toxoid, polyamino acid residues such as poly L-lysine, poly L-glutamic acid, influenza virus proteins, hepatitis B virus core protein, and the like.

In some embodiments, an antigen or neoantigen peptide described herein can be modified by terminal-NH₂ acylation, e.g., by alkanoyl (C₁-C₂₀) or thioglycolyl acetylation, terminal-carboxyl amidation, e.g., ammonia, methylamine, etc. In some embodiments these modifications can provide sites for linking to a support or other molecule.

In some embodiments, an antigen or neoantigen peptide described herein can contain modifications such as but not limited to glycosylation, side chain oxidation, biotinylation, phosphorylation, addition of a surface active material, e.g. a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

In some embodiments, an antigen or neoantigen peptide described herein can contain substitutions to modify a physical property (e.g., stability or solubility) of the resulting peptide. For example, an antigen or neoantigen peptide can be modified by the substitution of a cysteine (C) with α-amino butyric acid (“B”). Due to its chemical nature, cysteine has the propensity to form disulfide bridges and sufficiently alter the peptide structurally so as to reduce binding capacity. Substituting α-amino butyric acid for C not only alleviates this problem, but actually improves binding and crossbinding capability in certain instances. Substitution of cysteine with α-amino butyric acid can occur at any residue of an antigen or neoantigen peptide, e.g., at either anchor or non-anchor positions of an epitope or analog within a peptide, or at other positions of a peptide.

In some embodiments, an antigen peptide or neoantigen peptide described herein can comprise amino acid mimetics or unnatural amino acid residues, e.g. D- or L-naphtylalanine; D- or L-phenylglycine; D- or L-2-thieneylalanine; D- or L-1, 2, 3, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoro-methyl)-phenylalanine; D-p-fluorophenylalanine; D- or L-p-biphenyl-phenylalanine; D- or L-p-methoxybiphenylphenylalanine; D- or L-2-indole(allyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acid residues. Aromatic rings of a non-natural amino acid include, e.g., thiazolyl, thiophenyl, pyrazolyl, benzimidazolyl, naphthyl, furanyl, pyrrolyl, and pyridyl aromatic rings. Modified peptides that have various amino acid mimetics or unnatural amino acid residues are particularly useful, as they tend to manifest increased stability in vivo. Such peptides can also possess improved shelf-life or manufacturing properties.

In some embodiments, the peptides are contacted to immune cells to activate the cells and make them antigen responsive.

In some embodiments, the peptides are contacted to immune cells ex vivo.

In some embodiments, the peptides are contacted to immune cells in the living system, e.g., a human being.

In some embodiments, the immune cells are antigen presenting cells.

In some embodiments, the immune cells are T cells.

The present disclosure relates to methods for manufacturing T cells which are specific to immunogenic antigens.

The present disclosure also relates to compositions comprising antigen specific T cells stimulated with APCs. In some embodiments, one or more antigen peptides are loaded on to APCs, wherein the peptide loaded APCs are then used to stimulate T cells to produce antigen specific T cells. In some embodiments, the antigens are neoantigens. In some embodiments, the APCs used for peptide loading are dendritic cells.

In some embodiments, a peptide sequence comprises a mutation that is not present in non-cancer cells of a subject. In In some embodiments, a peptide is encoded by a gene or an expressed gene of a subject's cancer cells. In some embodiments, a peptide sequence has a length of at least 8; 9; 10; 11; 12; 13; 14; 15; 16; 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 or more naturally occurring amino acids.

In some embodiments, a peptide sequence binds to a protein encoded by a class I HLA allele and has a length of from 8-12 naturally occurring amino acids. In some embodiments, a peptide sequence binds to a protein encoded by a class II HLA allele and has a length of from 16-25 naturally occurring amino acids. In some embodiments, a peptide sequence comprises a plurality of peptide antigen sequences. In some embodiments, the plurality of peptide antigen sequences comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, or 500 peptide antigen sequences.

In some embodiments, the antigens described herein are neoantigens. Candidate immunogenic neoantigen sequences can be identified by any suitable method known in the art. The methods of the present disclosure can be useful, for example, to produce therapies specific to a subject's disease or to produce vaccines to a disease. Candidate immunogenic neoantigens can be neoantigens previously identified. In some embodiments, candidate immunogenic neoantigens may not be previously identified. Candidate immunogenic neoantigens for use in the methods and compositions described herein can be specific to a subject. In some embodiments, candidate neoantigens for use in the methods and compositions described herein can be specific to a plurality of subjects.

In both animals and humans, mutated epitopes can be potentially effective in inducing an immune response or activating T cells. In one embodiment, the potentially immunogenic epitopes of an infectious agent in a subject, such as a virus, can be determined. In one embodiment, the potentially immunogenic mutated epitopes of a subject with a disease, such as cancer, can be determined. In some embodiments, a potentially immunogenic antigen or neoantigen for use in the methods described herein can be a differentiation antigen expressed in a tumor and cells of the type of tissue from which they are generated. In some embodiments, a potentially immunogenic antigen or neoantigen for use in the methods described herein can be a cancer/germ line antigens not expressed in another differentiated tissue. In some embodiments, a potentially immunogenic antigen or neoantigen for use in the methods described herein can be a mutated antigen. For example, a candidate immunogenic antigen or neoantigen peptide for use in the methods described herein can comprise a missense point mutation or an antigen or neoantigen of a fusion protein generated through tumor specific translocation of a gene segment. In some embodiments, a potentially immunogenic antigen or neoantigen for use in the methods described herein can be an overexpressed antigen. In some embodiments, a potentially immunogenic antigen or neoantigen can be found in tumors. For example, a potentially immunogenic antigen or neoantigen for use in the methods described herein can include a protein whose expression is strictly regulated in cells of differentiated normal tissue.

Potentially immunogenic mutated epitopes can be determined by genomic or exomic sequencing of tumor tissue and healthy tissue from a cancer patient using next generation sequencing technologies. For example, genes selected based on their mutation frequency and ability to act as an antigen or neoantigen can be sequenced using next generation sequencing technology. In one embodiment, sequencing data can be analyzed to identify potentially immunogenic mutated peptides that can bind to HLA molecules of the subject. In one embodiment, the data can be analyzed using a computer. In another embodiment the sequence data can be analyzed for the presence of antigen or neoantigen peptides. In one embodiment, potentially immunogenic antigen or neoantigen peptides can be determined by their affinity to MHC molecules.

Potentially immunogenic antigen or neoantigen peptides can be determined by direct protein sequencing. For example, protein sequencing of enzymatic protein digests using multidimensional mass spectrometry techniques (e.g., tandem mass spectrometry (MS/MS)) can be used to identify potentially immunogenic antigen or neoantigen peptides for use in the methods described herein.

High-throughput methods for de novo sequencing of unknown proteins may be used to identify potentially immunogenic antigen or neoantigen peptides. For example, high-throughput methods for de novo sequencing of unknown proteins, such as meta-shotgun protein sequencing, may be used to analyze the proteome of a subject's tumor to identify potentially immunogenic expressed neoantigens.

Potentially immunogenic antigen or neoantigen peptides may also be identified using MHC multimers to identify antigen-specific T cell responses. For example, high-throughput analysis of antigen-specific T cell responses in patient samples may be performed using MHC tetramer-based screening techniques. Tetramer-based screening techniques may be used for the initial identification of potentially immunogenic tumor specific antigens, or alternatively as a secondary screening protocol to assess what potentially immunogenic antigens a patient may have already been exposed to, thereby facilitating the selection of potentially immunogenic antigens for use in the methods described herein.

In some embodiments, specific neoantigens are targeted for immunotherapy. In some embodiments, neoantigenic peptides are synthesized. The neoantigenic peptides used herein are designed such that each peptide is specific for an HLA antigen and can bind to the HLA antigen with a high binding affinity and specificity. In some embodiments, the peptides used herein are designed based on a high performance HLA binding prediction model generated by the inventors, and have been described in, for example the following patent applications/publications: WO2011143656, WO2017184590, and U.S. provisional application Nos. 62/783,914 and 62/826,827; all of which are incorporated by reference herein. NetMHCIIpan may be the current prediction standard, but it may not be regarded as accurate. Of the three Class II loci (DR, DP, and DQ), data may only exist for certain common alleles of HLA-DR. Briefly, the newly generated prediction model helps identify immunogenic antigen peptides and can be used to develop drugs, such as personalized medicine drugs, and isolation and characterization of antigen-specific T cells, wherein the machine-learning HLA-peptide presentation prediction model comprises, a plurality of predictor variables identified at least based on training data wherein the training data comprises: sequence information of sequences of peptides presented by a HLA protein expressed in cells and identified by mass spectrometry; training peptide sequence information comprising amino acid position information, wherein the training peptide sequence information is associated with the HLA protein expressed in cells; and a function representing a relation between the amino acid position information received as input and the presentation likelihood generated as output based on the amino acid position information and the predictor variables. CD4+ T cell responses may have anti-tumor activity. In existing prediction methods high rate of CD4+ T cell responses may be shown without using Class II prediction (e.g., 60% of SLP epitopes in NeoVax study (49% in NT-001), and 48% of mRNA epitopes in BioNTech study). It may not be clear whether these epitopes are typically presented natively (by tumor or by phagocytic DCs). It was therefore desirable to translate high CD4+T response rates into therapeutic efficacy by improving identification of naturally presented Class II epitopes. The roles of gene expression, enzymatic cleavage, and pathway/localization bias may have not been robustly quantified. It may be unclear whether autophagy (Class II presentation by tumor cells) or phagocytosis (Class II presentation of tumor epitopes by APCs) is the more relevant pathway, although most existing MS data may be presumed to derive from autophagy. There may be different data generation approaches for learning the rules of Class II presentation, including the field standard and the proposed approach. The field standard may comprise affinity measurements, which may be the basis for the NetMHCIIpan predictor, providing low throughput and requiring radioactive reagents, and it misses the role of processing. The new approach comprises mass spectrometry, where data from cell lines/tissues/tumors may help determine processing rules for autophagy (much of this data is already published) and Mono-allelic MS may enable determination of allele-specific binding rules (multi-allelic MS data is presumed overly complex for efficient learning. The newly generated prediction method comprises training a machine-learning HLA-peptide presentation prediction model, wherein training comprises inputting amino acid position information sequences of HLA-peptides isolated from one or more HLA-peptide complexes from a cell expressing a HLA class II allele into the HLA-peptide presentation prediction model using a computer processor; the machine-learning HLA-peptide presentation prediction model comprising: a plurality of predictor variables identified at least based on training data that comprises: sequence information of sequences of peptides presented by a HLA protein expressed in cells and identified by mass spectrometry; training peptide sequence information comprising amino acid position information of training peptides, wherein the training peptide sequence information is associated with the HLA protein expressed in cells; and a function representing a relation between the amino acid position information received as input and a presentation likelihood generated as output based on the amino acid position information and the predictor variables. In some embodiments, the presentation model has a positive predictive value of at least 0.25 at a recall rate of from 0.1%-10%. In some embodiments, the presentation model has a positive predictive value of at least 0.4 at a recall rate of from 0.1%-10%. In some embodiments, the presentation model has a positive predictive value of at least 0.6 at a recall rate of from 0.1%-10%. In some embodiments, the mass spectrometry is mono-allelic mass spectrometry. In some embodiments, the peptides are presented by a HLA protein expressed in cells through autophagy. In some embodiments, the peptides are presented by a HLA protein expressed in cells through phagocytosis. In some embodiments, the quality of the training data is increased by using a plurality of quality metrics. In some embodiments, the plurality of quality metrics comprises common contaminant peptide removal, high scored peak intensity, high score, and high mass accuracy. In some embodiments, the scored peak intensity is at least 50%. In some embodiments, the scored peak intensity is at least 70%. In some embodiments, the peptides presented by a HLA protein expressed in cells are peptides presented by a single immunoprecipitated HLA protein expressed in cells. In some embodiments, the plurality of predictor variables comprises a peptide-HLA affinity predictor variable. In some embodiments, the plurality of predictor variables comprises a source protein expression level predictor variable. In some embodiments, the plurality of predictor variables comprises a peptide cleavability predictor variable. In some embodiments, the peptides presented by the HLA protein comprise peptides identified by searching a peptide database using a reversed-database search strategy. In some embodiments, the HLA protein is an HLA-DR, and HLA-DP or an HLA-DQ protein. In some embodiments, the HLA protein is an HLA-DR protein selected from the group consisting of an HLA-DR, and HLA-DP or an HLA-DQ protein. In some embodiments, the HLA protein is an HLA-DR protein selected from the group consisting of: HLA-DPB1*01:01/HLA-DPA1*01:03, HLA-DPB1*02:01/HLA-DPA1*01:03, HLA-DPB1*03:01/HLA-DPA1*01:03, HLA-DPB1*04:01/HLA-DPA1*01:03, HLA-DPB1*04:02/HLA-DPA1*01:03, HLA-DPB1*06:01/HLA-DPA1*01:03, HLA-DQB1*02:01/HLA-DQA1*05:01, HLA-DQB1*02:02/HLA-DQA1*02:01, HLA-DQB1*06:02/HLA-DQA1*01:02, HLA-DQB1*06:04/HLA-DQA1*01:02, HLA-DRB1*01:01, HLA-DRB1*01:02, HLA-DRB1*03:01, HLA-DRB1*03:02, HLA-DRB1*04:01, HLA-DRB1*04:02, HLA-DRB1*04:03, HLA-DRB1*04:04, HLA-DRB1*04:05, HLA-DRB1*04:07, HLA-DRB1*07:01, HLA-DRB1*08:01, HLA-DRB1*08:02, HLA-DRB1*08:03, HLA-DRB1*08:04, HLA-DRB1*09:01, HLA-DRB1*10:01, HLA-DRB1*11:01, HLA-DRB1*11:02, HLA-DRB1*11:04, HLA-DRB1*12:01, HLA-DRB1*12:02, HLA-DRB1*13:01, HLA-DRB1*13:02, HLA-DRB1*13:03, HLA-DRB1*14:01, HLA-DRB1*15:01, HLA-DRB1*15:02, HLA-DRB1*15:03, HLA-DRB1*16:01, HLA-DRB3*01:01, HLA-DRB3*02:02, HLA-DRB3*03:01, HLA-DRB4*01:01, and HLA-DRB5*01:01. In some embodiments, the peptides presented by the HLA protein comprise peptides identified by comparing MS/MS spectra of the HLA-peptides with MS/MS spectra of one or more HLA-peptides in a peptide database.

In some embodiments, the mutation is selected from the group consisting of a point mutation, a splice site mutation, a frameshift mutation, a read-through mutation, and a gene fusion mutation.

In some embodiments, the peptides presented by the HLA protein have a length of 15-40 amino acids. In some embodiments, the peptides presented by the HLA protein comprise peptides identified by (a) isolating one or more HLA complexes from a cell line expressing a single HLA class II allele; (b) isolating one or more HLA-peptides from the one or more isolated HLA complexes; (c) obtaining MS/MS spectra for the one or more isolated HLA-peptides; and (d) obtaining a peptide sequence that corresponds to the MS/MS spectra of the one or more isolated HLA-peptides from a peptide database; wherein one or more sequences obtained from step (d) identifies the sequence of the one or more isolated HLA-peptides.

Various antigen peptides can be used to induce or expand T cells. Various antigen peptides can be used to activate antigen presenting cells (APCs), which in turn activate the T cells by contacting the T cells with antigen loaded APCs.

In some embodiments, a peptide comprises a mutation selected from (A) a point mutation, (B) a splice-site mutation, (C) a frameshift mutation, (D) a read-through mutation, (E) a gene-fusion mutation, and combinations thereof. In some embodiments, a peptide comprises a point mutation and binds to the HLA protein of a subject with a greater affinity than a corresponding wild-type peptide.

In some embodiments, a peptide binds to the HLA protein of a subject with an IC₅₀ of less than 500 nM, 250 nM, 150 nM, 100 nM, 50 nM, 25 nM or 10 nM. In some embodiments, a peptide binds to the HLA protein of a subject with an IC₅₀ or a K_{D} of less than 500 nM, 250 nM, 150 nM, 100 nM, 50 nM, 25 nM or 10 nM. In some embodiments, each peptide binds to a protein encoded by an HLA allele expressed by a subject. In some embodiments, a TCR of an antigen specific T cell induced or expanded binds to a peptide-HLA complex with an IC₅₀ or a K_{D} of less than 500 nM, 250 nM, 150 nM, 100 nM, 50 nM, 25 nM or 10 nM. In some embodiments, the TCR binds to an peptide-HLA complex with an IC₅₀ or a K_{D} of less than 500 nM, 250 nM, 150 nM, 100 nM, 50 nM, 25 nM or 10 nM. In some embodiments, each of the at least one peptide antigen sequences comprises a mutation that is not present in non-cancer cells of a subject. In some embodiments, each of the at least one peptide antigen sequences is encoded by gene or an expressed gene of a subject's cancer cells.

In some embodiments, a peptide has a length of at least 8; 9; 10; 11; 12; 13; 14; 15; 16; 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 or more naturally occurring amino acids. In some embodiments, a peptide binds to a protein encoded by a class I HLA allele and has a length of from 8-12 naturally occurring amino acids. In some embodiments, a peptide binds to a protein encoded by a class II HLA allele and has a length of from 16-25 naturally occurring amino acids. In some embodiments, a peptide comprises a plurality of peptides. In some embodiments, the plurality of peptides comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, or 500 or more antigen peptides.

In some aspects, the present disclosure provides peptides or polynucleotides encoding peptides identified using the methods described briefly above herein (e.g., a peptide with a tumor specific mutation, a viral peptide, or peptide associated with a non-cancerous disease).

In some embodiments, an optical method is used to select or identify immunogenic antigens. In some embodiments, a barcoded probe is used to select or identify immunogenic antigens. In some embodiments, a barcoded probe comprising a target specific region and a barcoded region is used to select or identify immunogenic antigens. In some embodiments the target specific region comprises a nucleic acid sequence that hybridizes to or has at least about 90%, 95% or 100% sequence complementarity to a nucleic acid sequence of a target polynucleotide.

### Preparing Activated, Antigen-Specific T Cells

Provided herein are methods for stimulating T cells. For example, the methods provided herein can be used to stimulate antigen specific T cells. The methods provided herein can be used to induce or activate T cells. For example, the methods provided herein can be used to expand activated T cells. For example, the methods provided herein can be used to induce naïve T cells. For example, the methods provided herein can be used to expand antigen specific CD8⁺ T cells. For example, the methods provided herein can be used to expand antigen specific CD4⁺ T cells. For example, the methods provided herein can be used to expand antigen specific CD8⁺ T cells having memory phenotype. For example, the therapeutic compositions can comprise antigen specific CD8+ T cells. For example, the therapeutic compositions can comprise antigen specific memory T cells.

T cells can be activated ex vivo with a composition comprising neoantigenic peptides or polynucleotides encoding the neoantigenic peptides.

T cells can be activated ex vivo with a composition comprising antigen loaded antigen presenting cells.

In some embodiments, the APCs and/or T cells are derived from a biological sample which is obtained from a subject.

In some embodiments, the APCs and/or T cells are derived from a biological sample which is peripheral blood mononuclear cells (PBMC).

In some embodiments, the subject is administered FLT3L prior to obtaining the biological sample for preparing the APCs and/or T cells.

In some embodiments, the APCs and/or T cells are derived from a biological sample which is a leukapheresis sample.

In some embodiments antigen presenting cells are first loaded with neoantigenic peptides ex vivo and used to prepare neoantigen activated T cells. In some embodiments, the compositions provided herein comprise T cells that are stimulated by APCs, such as APCs pre-loaded with antigen peptides. The compositions can comprise a population of immune cells comprising T cells from a sample (e.g., a biological sample), wherein the T cells comprise APC-stimulated T cells. In some embodiments, mRNA encoding one or more neoantigenic peptides are introduced into APCs for expression of the neoantigenic peptides. Such APCs are used for stimulating or activating T cells.

In some embodiments, the biological sample comprises a percentage of the at least one antigen specific T cell in the composition that is at least about 0.00001%, 0.00002%, 0.00005%, 0.0001%, 0.0005%, 0.001%, 0.005%, 0.01%, 0.05%, 0.1%, 0.5% of the total cell number. In some embodiments, the biological sample comprises less than 0.0001%, 0.0005%, 0.001%, 0.005%, 0.01%, 0.05%, 0.1%, 0.5%. 1%, 2%, 3%, 4%, 5%, or less than 10% antigen activated T cells of the total cell count in the biological sample that is derived from peripheral blood or leukapheresis. In some embodiments, the biological sample comprises less than 1%, 2%, 3%, 4%, 5%, 6%, 7%, 8%, 9%, 10%, 11%, 12%, 13%, 14%, 15%, 16%, 17%, 18%, 19%, 20%, 25%, 30% antigen activated T cells of the total cell count in the biological sample that is derived from peripheral blood or leukapheresis.

In some embodiments, the biological sample comprises antigen naive T cells. In some embodiments, the biological sample comprises greater than about 0.00001%, 0.00002%, 0.00005%, 0.0001%, 0.0005%, 0.001%, 0.005%, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% antigen naive cells of the total cell count in the biological sample that is derived from peripheral blood or leukapheresis.

In some embodiments, a percentage of at least one antigen specific CD8+ T cell in the composition is less than about 0.00001%, 0.00002%, 0.00005%, 0.0001%, 0.0005%, 0.001%, 0.005%, 0.01%, 0.05%, 0.1%, 0.5%, 1%, 2%, 3%, 4%, 5% in the biological sample derived from peripheral blood or leukapheresis. In some embodiments, a percentage of at least one antigen specific CD4+ T cell in the composition is at least about 0.00001%, 0.00002%, 0.00005%, 0.0001%, 0.0005%, 0.001%, 0.005%, 0.01%, 0.05%, 0.1%, 0.5%, 1%, 2%, 3%, 4%, 5%, 6%, 7%, 8%, 9%, 10%, of in the biological sample derived from peripheral blood or leukapheresis.

In some embodiments, a percentage of the at least one antigen specific T cell in the biological sample is at most about 0.00001%, 0.00005%, 0.0001%, 0.0005%, 0.001%, 0.005%, 0.01%, 0.05%, 0.1% or 0.5% of the total immune cells. In some embodiments, a percentage of at least one antigen specific CD8⁺ T cell in the biological sample is at most about 0.00001%, 0.00005%, 0.0001%, 0.0005%, 0.001%, 0.005%, 0.01%, 0.05%, 0.1% or 0.5% of the total immune cells. In some embodiments, a percentage of at least one antigen specific CD4⁺ T cell in the biological sample is at most about 0.00001%, 0.00005%, 0.0001%, 0.0005%, 0.001%, 0.005%, 0.01%, 0.05%, 0.1% or 0.5% of the total immune cells. In some embodiments, a percentage of antigen specific T cells in the biological sample is at most about 0.5%. In some embodiments, a percentage of neoantigen specific CD8⁺ T cells in the biological sample is at most about 0.5%. In some embodiments, a percentage of antigen specific CD4⁺ T cells in the biological sample is at most about 0.5% in the biological sample.

### Preparing Neoantigen Loaded APCs

In some embodiments, a composition comprises a population of immune cells that has been incubated with one or more cytokines, growth factors or ligands, such as a ligand that binds to a cell surface receptor of an APC or a T cell. Non-limiting examples of such cytokines, growth factors and ligands include, but are not limited to, GM-CSF, IL-4, IL-7, FLT3L, TNF-α, IL-1β, IL-15, PGE1, IL-6, IFN-α, IFN-γ, R848, LPS, ss-rna40, and polyI:C. In some embodiments, a composition comprises a population of immune cells that has been incubated with one or more APCs or APC preparations. For example, a composition can comprise a population of immune cells that has been incubated with one or more cytokine, growth factor and/or ligand stimulated APCs or cytokine, growth factor and/or ligand stimulated APC preparations. For example, a composition can comprise a population of immune cells that has been incubated with one or more cytokine stimulated APCs or cytokine stimulated APC preparations. For example, a composition can comprise a population of immune cells that have been incubated with one or more growth factor stimulated APCs or growth factor stimulated APC preparations. For example, a composition can comprise a population of immune cells that has been incubated with one or more ligand stimulated APCs or ligand stimulated APC preparations.

In some embodiments, the APC is an autologous APC, an allogenic APC, or an artificial APC.

Immune cells are characterized by cell surface molecules. In some embodiments the immune cells are preferably selected based on the cell surface markers, for example, from the biological sample, by using antibodies that can bind to the cell surface receptors. In some embodiments some cells are negatively selected to enrich one or more cell types that do not express the cell surface molecule that they are negatively selected for.

In some embodiments, antigen presenting cells (APCs) are prepared from the biological sample by selecting from APCs or precursor cells that can be cultured in presence of neoantigenic peptides to generate neoantigen-loaded APCs, which are used for activating T cells. Some of the related cell surface markers for selecting and/or enriching for a set of cells is described below.

CD1 (cluster of differentiation 1) is a family of glycoproteins expressed on the surface of various human antigen-presenting cells. They are related to the class I MHC molecules, and are involved in the presentation of lipid antigens to T cells.

CD11b or Integrin alpha M (ITGAM) is one protein subunit that forms heterodimeric integrin alpha-M beta-2 (α_{M}β₂) molecule, also known as macrophage-1 antigen (Mac-1) or complement receptor 3(CR3). ITGAM is also known as CR3A, and cluster of differentiation molecule 11b (CD11b). The second chain of α_{M}β₂ is the common integrin β₂ subunit known as CD18, and integrin α_{M}β₂ thus belongs to the β₂ subfamily (or leukocyte) integrins. α_{M}β₂ is expressed on the surface of many leukocytes involved in the innate immune system, including monocytes, granulocytes, macrophages, and natural killer cells. It mediates inflammation by regulating leukocyte adhesion and migration and has been implicated in several immune processes such as phagocytosis, cell-mediated cytotoxicity, chemotaxis and cellular activation. It is involved in the complement system due to its capacity to bind inactivated complement component 3b (iC3b). The ITGAM (alpha) subunit of integrin α_{M}β₂ is directly involved in causing the adhesion and spreading of cells but cannot mediate cellular migration without the presence of the β₂ (CD18) subunit.

CD11c, also known as Integrin, alpha X (complement component 3 receptor 4 subunit) (ITGAX), is a gene that encodes for CD11c. CD11c is an integrin alpha X chain protein. Integrins are heterodimeric integral membrane proteins composed of an alpha chain and a beta chain. This protein combines with the beta 2 chain (ITGB2) to form a leukocyte-specific integrin referred to as inactivated-C3b (iC3b) receptor 4 (CR4). The alpha X beta 2 complex seems to overlap the properties of the alpha M beta 2 integrin in the adherence of neutrophils and monocytes to stimulated endothelium cells, and in the phagocytosis of complement coated particles. CD11c is a type I transmembrane protein found at high levels on most human dendritic cells, but also on monocytes, macrophages, neutrophils, and some B cells that induces cellular activation and helps trigger neutrophil respiratory burst; expressed in hairy cell leukemias, acute nonlymphocytic leukemias, and some B-cell chronic lymphocytic leukemias.

CD14 is a surface antigen that is preferentially expressed on monocytes/macrophages. It cooperates with other proteins to mediate the innate immune response to bacterial lipopolysaccharide. Alternative splicing results in multiple transcript variants encoding the same protein. CD14 exists in two forms, one anchored to the membrane by a glycosylphosphatidylinositol tail (mCD14), the other a soluble form (sCD14). Soluble CD14 either appears after shedding of mCD14 (48 kDa) or is directly secreted from intracellular vesicles (56 kDa). CD14 acts as a co-receptor (along with the Toll-like receptor TLR 4 and MD-2) for the detection of bacterial lipopolysaccharide (LPS). CD14 can bind LPS only in the presence of lipopolysaccharide-binding protein (LBP). Although LPS is considered its main ligand, CD14 also recognizes other pathogen-associated molecular patterns such as lipoteichoic acid.

CD25 is expressed by conventional T cells after stimulation, and it has been shown that in human peripheral blood, only the CD4⁺ CD25^{hi} T cells are ‘suppressors’.

In some embodiments, the APC comprises a dendritic cell (DC). In some embodiments, the APC is derived from a CD14⁺ monocyte. In some embodiments, the APCs can be obtained from skin, spleen, bone marrow, thymus, lymph nodes, peripheral blood, or cord blood. In some embodiments, the CD14⁺ monocyte is from a biological sample from a subject comprising PBMCs. For example, a CD14⁺ monocyte can be isolated from, enriched from, or purified from a biological sample from a subject comprising PBMCs. In some embodiments, the CD14⁺ monocyte is stimulated with one or more cytokines or growth factors. In some embodiments, the one or more cytokines or growth factors comprise GM-CSF, IL-4, FLT3L, TNF-α, IL-1β, PGE1, IL-6, IL-7, IL-15, IFNγ, IFN-α, R848, LPS, ss-rna40, poly I:C, or a combination thereof. In some embodiments, the CD14⁺ monocyte is from a second biological sample comprising PBMCs.

In some embodiments, an isolated population of APCs can be enriched or substantially enriched. In some embodiments, the isolated population of APCs is at least 30%, at least 50%, at least 75%, or at least 90% homogeneous. In some embodiments, the isolated population of APCs is at least 60%, at least 75%, or at least 90% homogeneous. APCs, such as APCs can include, for example, APCs derived in culture from monocytic dendritic precursors as well as endogenously-derived APCs present in tissues such as, for example, peripheral blood, cord blood, skin, spleen, bone marrow, thymus, and lymph nodes.

APCs and cell populations substantially enriched for APCs can be isolated by methods also provided by the present invention. The methods generally include obtaining a population of cells that includes APC precursors, differentiation of the APC precursors into immature or mature APCs, and can also include the isolation of APCs from the population of differentiated immature or mature APCs.

APC precursor cells can be obtained by methods known in the art. APC precursors can be isolated, for example, by density gradient separation, fluorescence activated cell sorting (FACS), immunological cell separation techniques such as panning, complement lysis, rosetting, magnetic cell separation techniques, nylon wool separation, and combinations of such methods. Methods for immuno-selecting APCs include, for example, using antibodies to cell surface markers associated with APC precursors, such as anti-CD34 and/or anti-CD14 antibodies coupled to a substrate.

Enriched populations of APC precursors can also be obtained. For example, enriched populations of APC precursors can be isolated from a tissue source by selective removal of cells that adhere to a substrate. Using a tissue source such as, e.g., bone marrow or peripheral blood, adherent monocytes can be removed from cell preparations using a commercially-treated plastic substrate (e.g., beads or magnetic beads) to obtain a population enriched for nonadherent APC precursors.

Monocyte APC precursors can also be obtained from a tissue source by using an APC precursor-adhering substrate. For example, peripheral blood leukocytes isolated by, e.g., leukapheresis, are contacted with a monocytic APC precursor-adhering substrate having a high surface area to volume ratio and the adherent monocytic APC precursors are separated. In additional embodiments, the substrate coupled can be a particulate or fibrous substrate having a high surface-to-volume ratio, such as, for example, microbeads, microcarrier beads, pellets, granules, powder, capillary tubes, microvillous membrane, and the like. Further, the particulate or fibrous substrate can be glass, polystyrene, plastic, glass-coated polystyrene microbeads, and the like. In some embodiments, APCs are enriched in a cell population from a biological sample by depleting the cell population of CD14+ cells, CD25+ cells and/or CD56+ cells.

The APC precursors can also be cultured in vitro for differentiation and/or expansion. Methods for differentiation/expansion of APC precursors are known in the art. Generally, expansion can be achieved by culturing the precursors in the presence of at least one cytokine that induces APC (e.g., dendritic cell) differentiation/proliferation. Typically, these cytokines are granulocyte colony stimulating factor (G-CSF) or granulocyte/macrophage colony stimulating factor (GM-CSF). In addition, other agents can be used to inhibit proliferation and/or maturation of non-APC cell types in the culture, thereby further enriching the population of APC precursors. Typically, such agents include cytokines such as, e.g., IL-13, IL-4, or IL-15, and the like.

The isolated populations of APC precursors are cultured and differentiated to obtain immature or mature APCs. Suitable tissue culture media include, for example, but not limited to, AIM-V®, RPMI 1640, DMEM, X-VIVO, and the like. The tissue culture media is typically supplemented with amino acids, vitamins, divalent cations, and cytokines to promote differentiation of the precursors toward the APC phenotype. Typically, the differentiation-promoting cytokines are GM-CSF and/or IL-4.

Further, cultures of APC precursors during expansion, differentiation, and maturation to the APC phenotype can include plasma to promote the development of APCs. A typical plasma concentration is about 5%. In addition, where, for example, APC precursors are isolated by adherence to a substrate, plasma can be included in the culture media during the adherence step to promote the CD14⁺ phenotype early in culture. A typical plasma concentration during adherence is about 1% or more.

The monocytic APC precursors can be cultured for any suitable time. In certain embodiments, suitable culture times for the differentiation of precursors to immature APCs can be about 1 to about 10 days, e.g., about 4 to about 7 days. The differentiation of immature APCs from the precursors can be monitored by methods known to those skilled in the art, such as by the presence or absence of cell surface markers (e.g., CD11c⁺, CD83^{low}, CD86^{-/low}, HLA-DR⁺). Immature APCs can also be cultured in appropriate tissue culture medium to maintain the immature APCs in a state for further differentiation or antigen uptake, processing and presentation. For example, immature APCs can be maintained in the presence of GM-CSF and IL-4.

In some embodiments, APC precursors may be isolated prior to differentiation. In some embodiments, the isolated population may be enriched or substantially enriched for APC precursors. In some embodiments, APC precursors are isolated with a CD14 specific probe. In one exemplary embodiment, CD14 expressing cells are detected by FACS using a CD14 specific probe either directly conjugated to a fluorescent molecule (e.g., FITC or PE) or with a unlabeled antibody specific for CD14 and a labeled second antibody specific for the first antibody. CD14⁺ cells can also be separated from CD14^{low} and CD14⁻ cells by FACS sorting. Gating for CD14^{high} positivity can be determined in reference to CD14 staining on, e.g., PBMC-derived monocytes. Typically, the CD14 specific binding agent is, for example, an anti-CD14 antibody (e.g., monoclonal or antigen binding fragments thereof). A number of anti-CD14 antibodies suitable for use in the present invention are well known to the skilled artisan and many can be purchased commercially. Differentiation into immature APCs (CD14 negative) can take place following isolation.

In another embodiment, a CD14 specific probe is coupled to a substrate and the CD14⁺ cells are isolated by affinity selection. A population of cells that includes CD14⁺ cells is exposed to the coupled substrate and the CD14⁺ cells are allowed to specifically adhere. Non-adhering CD14⁻ cells are then washed from the substrate, and the adherent cells are then eluted to obtain an isolated cell population substantially enriched in APC precursors. The CD14 specific probe can be, for example, an anti-CD14 antibody. The substrate can be, for example, commercially available tissue culture plates or beads (e.g., glass or magnetic beads). Methods for affinity isolation of cell populations using substrate-coupled antibodies specific for surface markers are generally known.

During culture, immature APCs can optionally be exposed to a predetermined antigen. Suitable predetermined antigens can include any antigen for which T-cell modulation is desired. In one embodiment, immature APCs are cultured in the presence of prostate specific membrane antigen (PSMA) for cancer immunotherapy and/or tumor growth inhibition. Other antigens can include, for example, bacterial cells, viruses, partially purified or purified bacterial or viral antigens, tumor cells, tumor specific or tumor associated antigens (e.g., tumor cell lysate, tumor cell membrane preparations, isolated antigens from tumors, fusion proteins, liposomes, and the like), recombinant cells expressing an antigen on its surface, autoantigens, and any other antigen. Any of the antigens can also be presented as a peptide or recombinantly produced protein or portion thereof. Following contact with antigen, the cells can be cultured for any suitable time to allow antigen uptake and processing, to expand the population of antigen-specific APCs, and the like.

For example, in one embodiment, the immature APCs can be cultured following antigen uptake to promote maturation of the immature APCs into mature APCs that present antigen in the context of MHC molecules. Methods for APC maturation are known. Such maturation can be performed, for example, by culture in the presence of known maturation factors, such as cytokines (e.g., TNF-α, IL-1β, or CD40 ligand), bacterial products (e.g., LPS or BCG), and the like. The maturation of immature APCs to mature APCs can be monitored by methods known in the art, such as, for example by measuring the presence or absence of cell surface markers (e.g., upregulation of CD83, CD86, and MHC molecules) or testing for the expression of mature APC specific mRNA or proteins using, for example, an oligonucleotide array.

Optionally, the immature APCs can be cultured in an appropriate tissue culture medium to expand the cell population and/or maintain the immature APCs in state for further differentiation or antigen uptake. For example, immature APCs can be maintained and/or expanded in the presence of GM-CSF and IL-4. Also, the immature APCs can be cultured in the presence of anti-inflammatory molecules such as, for example, anti-inflammatory cytokines (e.g., IL-10 and TGF-β) to inhibit immature APC maturation.

In another aspect, the isolated population of APCs is enriched for mature APCs. The isolated population of mature APCs can be obtained by culturing a differentiated population of immature APCs in the presence of maturation factors as described above (e.g., bacterial products, and/or proinflammatory cytokines), thereby inducing maturation. Immature APCs can be isolated by removing CD14+ cells.

According to yet another aspect of the invention, APCs can be preserved, e.g., by cryopreservation either before exposure or following exposure to a suitable antigen. Cryopreservation agents which can be used include but are not limited to dimethyl sulfoxide (DMSO), glycerol, polyvinylpyrrolidone, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts. A controlled slow cooling rate can be critical. Different cryoprotective agents and different cell types typically have different optimal cooling rates. The heat of fusion phase where water turns to ice typically should be minimal. The cooling procedure can be carried out by use of, e.g., a programmable freezing device or a methanol bath procedure. Programmable freezing apparatuses allow determination of optimal cooling rates and facilitate standard reproducible cooling. Programmable controlled-rate freezers such as Cryomed or Planar permit tuning of the freezing regimen to the desired cooling rate curve.

After thorough freezing, APCs can be rapidly transferred to a long-term cryogenic storage vessel. In a typical embodiment, samples can be cryogenically stored in liquid nitrogen (−196° C.) or its vapor (−165° C.). Considerations and procedures for the manipulation, cryopreservation, and long term storage of hematopoietic stem cells, particularly from bone marrow or peripheral blood, is largely applicable to the APCs of the invention.

Frozen cells are preferably thawed quickly (e.g., in a water bath maintained at 37-41° C.) and chilled immediately upon thawing. It may be desirable to treat the cells in order to prevent cellular clumping upon thawing. To prevent clumping, various procedures can be used, including but not limited to the addition before and/or after freezing of DNAse, low molecular weight dextran and citrate, hydroxyethyl starch, and the like. The cryoprotective agent, if toxic in humans, should be removed prior to therapeutic use of the thawed APCs. One way in which to remove the cryoprotective agent is by dilution to an insignificant concentration. Once frozen APCs have been thawed and recovered, they can be used to activate T cells as described herein with respect to non-frozen APCs.

In one aspect, a composition for T cell activation comprises a population of immune cells that has been depleted of one or more types of immune cells. For example, a composition can comprise a population of immune cells that has been depleted of one or more types of immune cells that express one or more proteins, such as one or more cell surface receptors. In some embodiments, a composition comprises a population of immune cells from a biological sample comprising at least one antigen specific T cells comprising a T cell receptor (TCR) specific to at least one peptide antigen sequence, wherein an amount of CD14 and/or CD25 expressing immune cells in the population is proportionally different from an amount of immune cells expressing CD14 and/or CD25 in the biological sample. For example, a composition can comprise a population of immune cells from a biological sample comprising at least one antigen specific T cells comprising a T cell receptor (TCR) specific to at least one peptide antigen sequence, wherein an amount of CD14 expressing immune cells in the population is proportionally different from an amount of immune cells expressing CD14 in the biological sample. For example, a composition can comprise a population of immune cells from a biological sample comprising at least one antigen specific T cells comprising a T cell receptor (TCR) specific to at least one peptide antigen sequence, wherein an amount of CD25 expressing immune cells in the population is proportionally different from an amount of immune cells expressing CD25 in the biological sample. For example, a composition can comprise a population of immune cells from a biological sample comprising at least one antigen specific T cells comprising a T cell receptor (TCR) specific to at least one peptide antigen sequence, wherein an amount of CD14 and CD25 expressing immune cells in the population is proportionally different from an amount of immune cells expressing CD14 and CD25 in the biological sample. For example, a composition can comprise a population of immune cells from a biological sample, wherein an amount of immune cells expressing CD14 and CD25 in the population is proportionally less than an amount of immune cells expressing CD14 and CD25 in the biological sample.

Provided herein is a method for preparing a cellular composition for cancer immunotherapy, comprising: I. preparing antigen loaded antigen presenting cells (APC), comprising: (a) obtaining peripheral blood mononuclear cells (PBMC) from a subject pretreated with fins-like tyrosine kinase 3 ligand (FLT3L); (b) contacting the PBMCs ex vivo with: (i) a plurality of cancer neoantigen peptides, or one or more polynucleotides encoding the plurality of cancer neoantigen peptides, and wherein, each of the cancer neoantigen peptides or a portion thereof binds to a protein encoded by an HLA allele expressed in the subject, (ii) a stimulant for activating the cells, (iii) an agent promoting cell growth and maintenance ex vivo, thereby obtaining a cell population, and (iv) an agent for reducing or depleting CD11b+ cells from the cell population to obtain a CD11b^{low} or CD11b depleted antigen loaded APC; II. contacting isolated T cells with the CD11b^{low} or CD11b depleted antigen loaded APCs ex vivo; III. preparing antigen primed T cells for a cellular composition for cancer immunotherapy.

In some embodiments, the subject is pretreated with FLT3L at least about 1 hour, 2 hours, 3 hours, 4 hours, 5 hours, 6 hours, 12 hours, 1 day, 2 days, 3 days, 4 days, 5 days, 6 days, or 1 week before isolation of PBMC or leukapheresis. In some embodiments, the subject is pretreated with FLT3L at least about 1 week, 2 weeks, 3 weeks, 4 weeks, or 5 weeks before isolation of PBMC or leukapheresis.

In some embodiments, the cell population is enriched for CD11c+ cells. In some embodiments, the antigen loaded APC comprises dendritic cells (DCs). In some embodiments, the antigen loaded APC comprises plasmacytoid dendritic cells (pDCs). In some embodiments, the antigen loaded APC comprises CD1c+ DCs. In some embodiments, the antigen loaded APC comprises CD141+ DCs. In some embodiments, the cell population comprises macrophages. In some embodiments, the method further comprises reducing or depleting CD19+ cells from the cell population for activating or enriching neoantigen activated T cells. In some embodiments, the method further comprises reducing or depleting both CD11b+ and CD19+ cells from the cell population for activating or enriching neoantigen activated T cells.

In some embodiments, the method further comprises reducing or depleting CD14+ cells from the cell population for preparing and enriching antigen activated T cells. In some embodiments, the method further comprises reducing or depleting CD25+ cells from the cell population for preparing and enriching antigen activated T cells. In some embodiments, the method further comprises reducing or depleting one or more of CD19+, CD14+, CD25+ or CD11b+ cells from the cell population for activating or enriching neoantigen activated T cells.

In some embodiments the stimulant for activating the cells comprises FL3TL.

In some embodiments the agent promoting cell growth and maintenance ex vivo comprises a growth factor, a cytokine, an amino acid, a supplement or a combination thereof.

In some embodiments the antigen loaded APCs can stimulate T cells for 2, 3, 4, 5, 6, or 7 days.

In some embodiments, each of the plurality of cancer neoantigen peptides is 8-30 amino acids long.

In some embodiments, each of the plurality of neoantigenic peptide comprises a neoantigenic epitope. In some embodiments the plurality of cancer neoantigen peptides comprises 2, 3, 4, 5, 6, 7 or 8 neoantigenic peptides; and each of the plurality of neoantigenic peptides have the neoantigenic peptide characteristics as described in the previous section.

In some embodiments, the neoantigenic peptides used to prepare antigen loaded APCs are long peptides comprising at least 20 amino acids, or at least 30 amino acids or at least 40 amino acids or at least 50 amino acids, or any number of amino acids in between. In some embodiments, the neoantigenic peptides used to prepare antigen loaded APCs comprise the amino acids flanking on either side of the mutation that facilitate endogenous processing of the neoantigenic peptide for increased rate of presentation to a T cell.

A longer immunogenic peptide can be designed in several ways. In some embodiments, when HLA-binding peptides are predicted or known, a longer immunogenic peptide could consist of (1) individual binding peptides with extensions of 2-5 amino acids toward the N- and C-terminus of each corresponding gene product; or (2) a concatenation of some or all of the binding peptides with extended sequences for each. In other embodiments, when sequencing reveals a long (>10 residues) epitope sequence, e.g., a neoepitope present in a tumor (e.g. due to a frameshift, read-through or intron inclusion that leads to a novel peptide sequence), a longer neoantigen peptide could consist of the entire stretch of novel tumor-specific amino acids as either a single longer peptide or several overlapping longer peptides. In some embodiments, use of a longer peptide is presumed to allow for endogenous processing by patient cells and can lead to more effective antigen presentation and induction of T cell responses. In some embodiments, two or more peptides can be used, where the peptides overlap and are tiled over the long neoantigen peptide.

In some embodiments, each of the plurality of neoantigenic peptide comprises the same neoantigenic epitope. In some embodiments the plurality of neoantigenic peptide comprises more than one neoantigenic epitope.

In some embodiments the one or more polynucleotides encoding the plurality of cancer neoantigen peptides is DNA.

In some embodiments the one or more polynucleotides encoding the plurality of cancer neoantigen peptides is inserted in one or more mammalian expression vectors.

In some embodiments the one or more polynucleotides encoding the plurality of cancer neoantigen peptides is messenger RNA.

In some embodiments, the invention provides RNA, oligoribonucleotide, and polyribonucleotide molecules comprising a modified nucleoside.

In some embodiments, the invention provides gene therapy vectors comprising the RNA, oligoribonucleotide, and polyribonucleotide.

In some embodiments, the invention provides gene therapy methods and gene transcription silencing methods comprising same.

In some embodiments the polynucleotide encodes a single neoantigenic peptide.

In some embodiments the one polynucleotide encodes more than one neoantigenic peptide.

In some embodiments, the polynucleotide is messenger RNA. In some embodiments, each messenger RNA comprises coding sequence for two or more neoantigenic peptides in tandem.

In some embodiments each messenger RNA comprises a coding sequence for two, three, four, five, six, seven, eight, nine or ten or more neoantigenic peptides in tandem. Typically, an mRNA comprises a 5′-UTR, a protein coding region, and a 3′-UTR. mRNA only possesses limited half-life in cells and in vitro. In some embodiments, the mRNA is self-amplifying mRNA. In the context of the present invention, mRNA may be generated by in vitro transcription from a DNA template. The in vitro transcription methodology is known to the skilled person. For example, there is a variety of in vitro transcription kits commercially available.

The stability and translation efficiency of RNA may be modified. For example, RNA may be stabilized and its translation increased by one or more modifications having a stabilizing effects and/or increasing translation efficiency of RNA. Such modifications are described, for example, in PCT/EP2006/009448 incorporated herein by reference. In order to increase expression of the RNA used according to the present invention, it may be modified within the coding region, i.e. the sequence encoding the expressed peptide or protein, without altering the sequence of the expressed peptide or protein, so as to increase the GC-content to increase mRNA stability and to perform a codon optimization and, thus, enhance translation in cells.

In some embodiments, an mRNA can include multiple neoantigenic epitopes. In some embodiment, long polyribonucleotide sequences can be used, that can encode neo-ORFs, for example, mutated GATA3 sequences, encoding neo-ORFs. In some a mRNA of a large portion of, or even the entire coding region of a gene comprising sequences encoding neoantigenic peptides are delivered into an immune cell for endogenous processing and presentation of antigens.

In some embodiments, the coding sequence for each neoantigenic peptide is 24-120 nucleotides long.

In some embodiments, the mRNA is 50-10,000 nucleotides long. In some embodiments, the mRNA is 100-10,000 nucleotides long. In some embodiments, the mRNA is 200-10,000 nucleotides long. In some embodiments, the mRNA is 50-5,000 nucleotides long. In some embodiments, the mRNA is 100-5,000 nucleotides long. In some embodiments, the mRNA is 100-1,000 nucleotides long. In some embodiments, the mRNA is 300-800 nucleotides long. In some embodiments, the mRNA is 400-700 nucleotides long. In some embodiments, the mRNA is 450-600 nucleotides long. In some embodiments, the mRNA is at least 200 nucleotides long. In some embodiments the mRNA is greater than 250 nucleotides, greater than 300 nucleotides, greater than 350 nucleotides, greater than 400 nucleotides, greater than 450 nucleotides, greater than 500 nucleotides, greater than 550 nucleotides, greater than 600 nucleotides, greater than 650 nucleotides, greater than 700 nucleotides, greater than 750 nucleotides, greater than 800 nucleotides, greater than 850 nucleotides long, greater than 900 nucleotides long greater than 950 nucleotides long, greater than 1000 nucleotides long, greater than 2000 nucleotides long, greater than 3000 nucleotides long, greater than 4000 nucleotides long or greater than 5000 nucleotides long.

In some embodiments, mRNA encoding one or more neoantigenic peptide is modified, wherein the modification relates to the 5′-UTR. In some embodiments, the modification relates to providing an RNA with a 5′-cap or 5′-cap analog in the 5′-UTR. The term “5′-cap” refers to a cap structure found on the 5′-end of an mRNA molecule and generally consists of a guanosine nucleotide connected to the mRNA via an unusual 5′ to 5′ triphosphate linkage. In some embodiments, this guanosine is methylated at the 7-position. The term “conventional 5′-cap” refers to a naturally occurring RNA 5′-cap, to the 7-methylguanosine cap (m G). In the context of the present invention, the term “5′-cap” includes a 5′-cap analog that resembles the RNA cap structure and is modified to possess the ability to stabilize RNA and/or enhance translation of RNA if attached thereto, in vivo and/or in a cell. In some embodiments, mRNA is capped cotranscriptionally.

In some embodiments, the mRNA encoding one or more neoantigenic peptides comprise a 3′-UTR comprising a poly A tail. In some embodiments, the poly A tail is 100-200 bp long (SEQ ID NO: 184). In some embodiments, the poly A tail is longer than 20 nucleotides. In some embodiments, the poly A tail is longer than 50 nucleotides. In some embodiments, the poly A tail is longer than 60 nucleotides. In some embodiments, the poly A tail is longer than 70 nucleotides. In some embodiments, the poly A tail is longer than 80 nucleotides. In some embodiments, the poly A tail is longer than 90 nucleotides. In some embodiments, the poly A tail is longer than 100 nucleotides. In some embodiments, the poly A tail is longer than 110 nucleotides. In some embodiments, the poly A tail is longer than 120 nucleotides. In some embodiments, the poly A tail is longer than 130 nucleotides. In some embodiments, the poly A tail is longer than 140 nucleotides. In some embodiments, the poly A tail is longer than 150 nucleotides. In some embodiments, the poly A tail is longer than 160 nucleotides. In some embodiments, the poly A tail is longer than 170 nucleotides. In some embodiments, the poly A tail is longer than 180 nucleotides. In some embodiments, the poly A tail is longer than 190 nucleotides. In some embodiments, the poly A tail is longer than 200 nucleotides. In some embodiments, the poly A tail is longer than 210 nucleotides. In some embodiments, the poly A tail is longer than 220 nucleotides. In some embodiments, the poly A tail is longer than 230 nucleotides. In some embodiments, the poly A tail is longer than 100 nucleotides. In some embodiments, the poly A tail is longer than 240 nucleotides. In some embodiments, the poly A tail is longer than 100 nucleotides. In some embodiments, the poly A tail is about 250 nucleotides.

In some embodiments, the poly A tail comprises 100-250 adenosine units (SEQ ID NO: 185). In some embodiments, the poly A tail comprises 120-130 adenine units (SEQ ID NO: 186). In some embodiments, the poly A tail comprises 120 adenine units (SEQ ID NO: 187). In some embodiments, the poly A tail comprises 121 adenine units (SEQ ID NO: 188). In some embodiments, the poly A tail comprises 122 adenine units (SEQ ID NO: 189). In some embodiments, the poly A tail comprises 123 adenine units (SEQ ID NO: 190). In some embodiments, the poly A tail comprises 124 adenine units (SEQ ID NO: 191). In some embodiments, the poly A tail comprises 125 adenine units (SEQ ID NO: 192). In some embodiments, the poly A tail as 129 bases (SEQ ID NO: 193).

In some embodiments, the coding sequence for two consecutive neoantigenic peptides are separated by a spacer or linker.

In some embodiments, the spacer or linker comprises up to 5000 nucleotide residues. An exemplary spacer sequence is GGCGGCAGCGGCGGCGGCGGCAGCGGCGGC (SEQ ID NO: 1). Another exemplary spacer sequence is GGCGGCAGCCTGGGCGGCGGCGGCAGCGGC (SEQ ID NO: 2). Another exemplary spacer sequence is GGCGTCGGCACC (SEQ ID NO: 3). Another exemplary spacer sequence is CAGCTGGGCCTG (SEQ ID NO: 4). Another exemplary spacer is a sequence that encodes a lysine, such as AAA or AAG. Another exemplary spacer sequence is CAACTGGGATTG (SEQ ID NO: 5).

In some embodiments, the mRNA comprises one or more additional structures to enhance antigen epitope processing and presentation by APCs.

In some embodiments, the linker or spacer region may contain cleavage sites. The cleavage sites ensure cleavage of the protein product comprising strings of epitope sequences into separate epitope sequences for presentation. The preferred cleavage sites are placed adjacent to certain epitopes in order to avoid inadvertent cleavage of the epitopes within the sequences. In some embodiments, the design of epitopes and cleavage regions on the mRNA encoding strings of epitopes are non-random.

In certain embodiments, an mRNA encoding a neoantigen peptide of the invention is administered to a subject in need thereof. In some embodiments, the mRNA to be administered comprises at least one modified nucleoside-phosphate.

In some embodiments, T cells are activated with neoantigenic peptides by artificial antigen presenting cells. In some embodiments, artificial scaffolds are used to activate a T cells with neoantigenic peptides, the artificial scaffolds are loaded with neoantigenic peptides couples with an MHC antigen to which the neoantigenic peptide can bind with high affinity.

In some embodiments, the additional structures comprise encoding specific domains from the proteins selected from a group MITD, SP1, and 10th Fibronectin Domain: 10FnIII.

In some embodiments, the cells derived from peripheral blood or from leukapheresis are contacted with the plurality of cancer neoantigen peptides, or one or more polynucleotides encoding the plurality of cancer neoantigen peptides once or more than once to prepare the antigen loaded APCs.

In some embodiments, the method comprises incubating the APC or one or more of the APC preparations with a first medium comprising at least one cytokine or growth factor for a first time period.

In some embodiments, the method comprises incubating one or more of the APC preparations with at least one peptide for a second time period.

In some embodiments, the enriched cells further comprise CD1c+ cells.

In some embodiments, the cell population is enriched for CD11c+ and CD141+ cells.

In some embodiments, the cell population comprising the antigen loaded APCs comprises greater than 1%, 2%, 3%, 4%, 5%, 6, 7%, 8%, 9%, 10%, 15%, 20%, 25%, 30% 35%, 40%, 45%, 50%, 55%, 60%, 65%, 70%, 75%, 80%, 85%, 90%, or 95% or more CD11c+ cells.

In some embodiments, the cell population comprising the antigen loaded APCs comprises less than 95%, 90%, 85%, 80%, 75%, 70%, 65%, 60%, 55%, 50%, 45%, 40%, 35%, 30%, 20%, 10%, 8%, 7%, 6%, 5%, 4% or lower CD11b+ expressing cells.

In some embodiments, the cell population comprising the antigen loaded APCs comprises greater than 1%, 2%, 3%, 4%, 5%, 6, 7%, 8%, 9%, 10%, 15%, 20%, 25%, 30% 35%, 40%, 45%, 50%, 55%, 60%, 65%, 70%, 75%, 80%, 85%, 90%, or 95% neoantigenic peptide expressing cells that are CD11c+.

In some embodiments, the cell population comprising the antigen loaded APCs comprises greater than 1%, 2%, 3%, 4%, 5%, 6, 7%, 8%, 9%, 10%, 15%, 20%, 25%, 30% 35%, 40%, 45%, 50%, 55%, 60%, 65%, 70%, 75%, 80%, 85%, 90%, or 95% neoantigenic peptide expressing cells that are CD11c+CD1c+, or CD141+ cells.

In some embodiments, the neoantigen loaded APCs comprise mature APCs.

In some embodiments, the method comprises obtaining a biological sample from a subject comprising at least one APC and at least one PBMC or at least on T cell.

In some embodiments, the method comprises depleting cells expressing CD14 and/or CD25 and/or CD19 from a biological sample, thereby obtaining a CD14 and/or CD25 and/or CD19 cell depleted sample.

In some embodiments, the method comprises incubating a CD14 and/or CD25 and/or CD19 cell depleted sample with FLT3L for a first time period.

In some embodiments, the method comprises incubating at least one peptide with a CD14 and/or CD25 and/or CD19 cell depleted sample for a second time period, thereby obtaining a first matured APC peptide loaded sample.

### Preparing Neoantigen Activated T Cells Using Neoantigen Loaded APCs (NEOSTIM)

In some embodiments, the neoantigen loaded APC (APC) prepared by the methods described above is incubated with T cells to obtain antigen activated T cells. The method can comprise generating at least one antigen specific T cell where the antigen is a neoantigen. In some embodiments, the generating at least one antigen specific T cell comprises generating a plurality of antigen specific T cells.

In some embodiments, the T cells are obtained from a biological sample from a subject.

In some embodiments, the T cells are obtained from a biological sample from the same subject from whom the APCs are derived. In some embodiments, the T cells are obtained from a biological sample from a different subject than the subject from whom the APCs are derived.

In some embodiments, the APCs and/or T cells are derived from a biological sample which is peripheral blood mononuclear cells (PBMC). In some embodiments, the APCs and/or T cells are derived from a biological sample which is a leukapheresis sample.

In some embodiments, the APC comprises a dendritic cell (DC).

In some embodiments, the APC is derived from a CD14+ monocyte, or is a CD14 enriched APC, or is a CD141 enriched APC.

In some embodiments, the CD14+ monocyte is enriched from a biological sample from a subject comprising peripheral blood mononuclear cells (PBMCs).

In some embodiments, the APC is PBMC. In some embodiments, the PBMC is freshly isolated PBMC. In some embodiments the PBMC is frozen PBMC. In some embodiments, the PBMC is autologous PBMC isolated from the subject or the patient.

In some embodiments, the PBMC is loaded with antigens, where the antigens may be peptides or polypeptides or polynucleotides, such as mRNA, that encode the peptides and polypeptides. PBMCs (monocytes, DCs phagocytic cells) can take up antigens by phagocytosis and process and present them on the surface for T cell activation. Peptides or polypeptides loaded on the PBMCs may be supplemented with adjuvants to increase immunogenicity. In some embodiments, the PBMC is loaded with nucleic acid antigens. Nucleic acid antigens may be in the form of mRNA, comprising sequences encoding one or more antigens. In some embodiments, mRNA antigen loading does not require adjuvant supplementation, because, for example, RNA can act as a self-adjuvant.

In some embodiments, the CD14+ monocyte is stimulated with one or more cytokines or growth factors.

In some embodiments, one or more cytokines or growth factors comprise GM-CSF, IL-4, FLT3L, TNF-α, IL-1β, PGE1, IL-6, IL12, IL-7, IL-15, IFNγ, IFN-α, R848, LPS, ss-rna40, poly I:C, or a combination thereof.

In some embodiments, the CD14+ monocyte is from a second biological sample comprising PBMCs.

In some embodiments, the second biological sample is from the same subject.

In some embodiments, the biological sample comprises peripheral blood mononuclear cells (PBMCs).

In some embodiments, the at least one antigen-specific T cell is stimulated in a medium comprising IL-7, IL-15, an indoleamine 2,3-dioxygenase-1 (IDO) inhibitor, an anti-PD-1 antibody, IL-12, or a combination thereof.

In some embodiments, the IDO inhibitor is epacadostat, navoximod, 1-methyltryptophan, or a combination thereof.

In some embodiments, the subject is administered FLT3L prior to obtaining the biological sample for preparing the APCs and/or T cells.

In some embodiments, the T cells are obtained from a biological sample from a subject as described in the previous sections of this disclosure.

In some embodiments, the biological sample is freshly obtained from a subject or is a frozen sample.

In some embodiments, the incubating is in presence of at least one cytokine or growth factor, which comprises GM-CSF, IL-4, FLT3L, TNF-α, IL-1β, PGE1, IL-6, IL-7, IL-12, IL-15, IFN-γ, IFN-α, IL-15, R848, LPS, ss-rna40, poly I:C, or any combination thereof.

In some embodiments, a method comprises stimulating T cells with IL-7, IL-15, or a combination thereof. In some embodiments, a method comprises stimulating T cells with IL-7, IL-15, or a combination thereof, in the presence of an IDO inhibitor, a PD-1 antibody or IL-12. In some embodiments, the method further comprises administering the antigen specific T cells to a subject.

In some embodiments, the method comprises incubating the APC prepared as described in the previous sections with T cells in presence of a medium comprising the at least one cytokines or growth factor to generate neoantigen activated T cells.

In some embodiments, the incubating comprises incubating a first APC preparation of the APC preparations to the T cells for more than 7 days.

In some embodiments, the incubating comprises incubating a first APC preparation of the APC preparations to the T cells for more than 7, 8, 9, 10, 11, 12, 13, 14, 15, 16, 17, 18, 19, or 20 days.

In some embodiments, the first time period of the one or more time periods is about 1, 2 3, 4, 5, 6, 7, 8, or 9 days.

In some embodiments, a total time period of the separate time periods is less than 28 days. In some embodiments, a total time period of the separate time periods is from 20-27 days. In some embodiments, a total time period of the separate time periods is 15, 16, 17, 18, 19, 20, 21, 22, 23, 24, 25, 26, 27, 28, 29, 30, 31, 32, 33, 34, 35, 36, 37, 38, or 39 days.

In some embodiments, a method comprises incubating a first APC preparation of the APC preparations with the T cells for more than 7 days. In some embodiments, a method comprises incubating a first APC preparation of the APC preparations with the T cells for more than 7, 8, 9, 10, 11, 12, 13, 14, 15, 16, 17, 18, 19, or 20 days. In some embodiments, a method comprises incubating a first APC preparation of the APC preparations with the T cells for from 7-20, 8-20, 9-20, 10-20, 11-20, or 12-20 days. In some embodiments, a method comprises incubating a first APC preparation of the APC preparations with the T cells for about 10-15 days.

In some embodiments, a method comprises incubating a second APC preparation of the APC preparations to the T cells for 5-9 days. In some embodiments, a method comprises incubating a second APC preparation of the APC preparations to the T cells for 5, 6, 7, 8, or 9 days. In some embodiments, the method further comprises removing the one or more cytokines or growth factors of the second medium after the third time period and before a start of the fourth time period.

In some embodiments, a method comprises incubating a third APC preparation of the APC preparations to the T cells for 5-9 days. In some embodiments, the method comprises incubating a third APC preparation of the APC preparations to the T cells for 5, 6, 7, 8, or 9 days.

In some embodiments, the method comprises incubating a first APC preparation of the APC preparations with the T cells for about 1, 2, 3, 4, 5, 6, 7, 8, 9, 10, 11, 12, 13, 14, 15, 16, 17, 18, 19, 20, 21, or 22 days, incubating a second APC preparation of the APC preparations to the T cells for about 1, 2, 3, 4, 5, 6, 7, 8, 9, 10, 11, 12, 13, 14, 15, 16, 17, 18, 19, 20, 21, or 22 days, and incubating a third APC preparation of the APC preparations to the T cells for about 1, 2, 3, 4, 5, 6, 7, 8, 9, 10, 11, 12, 13, 14, 15, 16, 17, 18, 19, 20, 21, or 22 days.

In some embodiments, a manufacturing process from isolation of mononuclear cells from a subject to obtaining the drug product comprising activated T cell population is about 24-36 calendar days. In some embodiments, the manufacturing process may be completed by less than 36 calendar days, less than 35 calendar days, less than 34 calendar days, less than 33 calendar days, less than 32 calendar days, less than 31 calendar days, less than 30 calendar days, less than 29 calendar days or less than 28 calendar days. In some embodiments, the manufacturing process may be completed in 28 days. In some embodiments, the manufacturing process may be completed in 27 days. In some embodiments, the manufacturing process may be completed in 26 days. In some embodiments the drug product specific to a target may have an overall turnaround time of 5 to 6 weeks, starting with the receipt of the manufacturing order and ending with product release and package for shipment.

In some embodiments, the manufacturing process may have the capability to run at least 2-3 manufacturing runs from 2-3 different patients per month. In some embodiments, the manufacturing process may have the capability to run at least 4-6 manufacturing runs from 4-6 different patients per month. In some embodiments, the manufacturing process may have the capability to run at least 5-7 manufacturing runs from 5-7 different patients per month. In some embodiments, the manufacturing process may have the capability to run at least 6-8 manufacturing runs from 6-8 different patients per month. In some embodiments, the manufacturing process may have the capability to run at least 7-9 manufacturing runs from 7-9 different patients per month. In some embodiments, the manufacturing process may have the capability to run at least 8-10 manufacturing runs from 8-10 different patients per month.

In some embodiments, manufacturing is done to generate T cells for clinical application following standardized GMP (good manufacturing practice). The protocol and a GMP facility has to be approved by the respective regulatory authorities. The GMP involves preparing the cells under aseptic conditions, or at least as approved by the respective authority. In some embodiments, the aseptic conditions may be validated by a respective authorized personnel or body. In some embodiments, the T cell manufacturing protocol may be required to follow acceptance criteria for the product specifications.

In some embodiments, the method is performed ex vivo. In some embodiments, the T cells are cultured in a medium containing a cytokine. In some embodiments, an example of cytokines includes IL-7. In some embodiments, an example of cytokines includes IL-15. In some embodiments, an example of cytokines includes IL-7 and IL-15. In some embodiments, the T cells are cultured in a medium comprising IL-7, and/or IL-15. In some embodiments, the cytokine in a T cell culture or a medium has a final concentration of at least 0.05 ng/mL, 0.1 ng/mL, 0.2 ng/mL, 0.3 ng/mL, 0.4 ng/mL, 0.5 ng/mL, 0.8 ng/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 12 ng/mL, 15 ng/mL, 18 ng/mL, or 20 ng/mL. In some embodiments, the IL-7 in a T cell culture or a medium has a final concentration of at least 0.05 ng/mL, 0.1 ng/mL, 0.2 ng/mL, 0.3 ng/mL, 0.4 ng/mL, 0.5 ng/mL, 0.8 ng/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 12 ng/mL, 15 ng/mL, 18 ng/mL, or 20 ng/mL. In some embodiments, the IL-15 in a T cell culture or a medium has a final concentration of at least 0.05 ng/mL, 0.1 ng/mL, 0.2 ng/mL, 0.3 ng/mL, 0.4 ng/mL, 0.5 ng/mL, 0.8 ng/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 12 ng/mL, 15 ng/mL, 18 ng/mL, or 20 ng/mL. In some embodiments, the T cells are cultured in a medium further containing FLT3L. In some embodiments, the FLT3L in a T cell culture or a medium has a final concentration of in a T cell culture or a medium has a final concentration of at least 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 12 ng/mL, 15 ng/mL, 18 ng/mL, 20 ng/mL, 30 ng/mL, 40 ng/mL, 50 ng/mL, 60 ng/mL, 70 ng/mL, 80 ng/mL, 90 ng/mL, 100 ng/mL, or 200 ng/mL. In some embodiments, the T cells are incubated, induced, or stimulated in a medium containing FLT3L for a first period time. In some embodiments, the T cells are incubated, induced, or stimulated in a medium containing additionally added FLT3L for a second period time. In some embodiments, the T cells are incubated, induced, or stimulated in a medium containing additional added FLT3L for a third period time. In some embodiments, the T cells are incubated, induced, or stimulated in a medium containing additional added FLT3L for a fourth, a fifth, or a sixth period time, with freshly added FLT3L in each time period.

In some embodiments, the T cells are cultured in presence a neoantigen, e.g. a neoantigen presented by an APC, wherein the media comprises high potassium [K]⁺ content. In some embodiments, the T cells are cultured in presence of high [K]⁺ content in the media for at least a period of time during the incubation with APCs or T cells. In some embodiments, the [K]⁺ content in the media is altered for at least a period of time during the incubation with APCs or T cells. In some embodiments, the content in the media is kept constant over the period of T cell ex vivo culture. In some embodiments, the [K]⁺ content in the T cell culture medium is ≥5 mM. In some embodiments, the [K]⁺ content in the T cell culture medium is ≥6 mM. In some embodiments, the [K]⁺ content in the T cell culture medium is ≥7 mM. In some embodiments, the [K]⁺ content in the T cell culture medium is ≥8 mM. In some embodiments, the [K]⁺ content in the T cell culture medium is ≥9 mM. In some embodiments, the [K]⁺ content in the T cell culture medium is ≥10 mM. In some embodiments, the [K]⁺ content in the T cell culture medium is ≥11 mM. In some embodiments, the [K]⁺ content in the T cell culture medium is ≥12 mM. In some embodiments, the [K]⁺ content in the T cell culture medium is ≥13 mM. In some embodiments, the [K]⁺ content in the T cell culture medium is ≥14 mM. In some embodiments, the [K]⁺ content in the T cell culture medium is ≥15 mM. In some embodiments, the [K]⁺ content in the T cell culture medium is ≥16 mM. In some embodiments, the [K]⁺ content in the T cell culture medium is ≥17 mM. In some embodiments, the [K]⁺ content in the T cell culture medium is ≥18 mM. In some embodiments, the [K]⁺ content in the T cell culture medium is ≥19 mM. In some embodiments, the [K]⁺ content in the T cell culture medium is ≥20 mM. In some embodiments, the [K]⁺ content in the T cell culture medium is ≥22 mM. In some embodiments, the [K]⁺ content in the T cell culture medium is ≥25 mM. In some embodiments, the [K]⁺ content in the T cell culture medium is ≥30 mM. In some embodiments, the [K]⁺ content in the T cell culture medium is ≥35 mM. In some embodiments, the [K]⁺ content in the T cell culture medium is ≥40 mM. In some embodiments, the [K]⁺ content in the T cell culture medium is about 40 mM.

In some embodiments, the [K]⁺ content in the T cell culture medium is about 40 mM for at least a period of time during the incubation of T cells with neoantigen. In some embodiments, the neoantigen may be presented by the neoantigen loaded APCs. In some embodiments, the T cells in the presence of [K]⁺ are tested for T effector functions, CD8⁺ cytotoxicity, cytokine production, and for memory phenotype. In some embodiments, T cells are grown in the presence of high [K]⁺ express effector T cell phenotype. In some embodiments, T cells grown in presence of high [K]⁺ express memory cell marker. In some embodiments, T cells grown in presence of high [K]⁺ do not express T cell exhaustion markers.

In one embodiment, provided herein is a method for producing a therapeutic population of T cells comprising: (a) culturing T cells from a biological sample from a subject in a first cell culture medium comprising antigen presenting cells (APCs) to produce a first population of T cells, wherein the APCs present an epitope of a peptide antigen in complex with an MHC protein; (b) optionally, culturing the first population of T cells in a second cell culture medium to produce a second population of T cells; (c) enriching CD137 (4-1BB)-expressing T cells and/or CD69 expressing T cells from the first or second population of T cells to produce a third population of T cells; and (d) expanding the third population of T cells in a third cell culture medium to obtain a therapeutic population of T cells comprising antigen-specific T cells.

In some embodiments, the method for producing a therapeutic population of T cells comprising: (a) culturing T cells from a biological sample from a subject in a first cell culture medium comprising antigen presenting cells (APCs), wherein the APCs present an epitope of a peptide antigen in complex with an MHC protein; (b) culturing the first population of T cells in a second cell culture medium to produce a second population of T cells; (c) optionally, enriching CD137 (4-1BB)-expressing T cells and/or CD69 expressing T cells from the second population of T cells to produce a third population of T cells; and (d) expanding the second or third population of T cells in a third cell culture medium to obtain a therapeutic population of T cells comprising antigen-specific T cells; wherein the concentration of the peptide antigen in the third culture medium is at least 2-fold lower than the concentration of the peptide antigen in the first culture medium and/or second culture medium.

In some embodiments, the method comprises enriching CD137 (4-1BB)-expressing T cells and/or CD69 expressing T cells from the second population of T cells to produce a third population of T cells.

In some embodiments, enriching CD137 (4-1BB)-expressing T cells and/or CD69 expressing T cells from the second population of T cells begins 10 days after beginning the culturing of the T cells from a biological sample from a subject in a first cell culture medium. In some embodiments, enriching CD137 (4-1BB)-expressing T cells and/or CD69 expressing T cells from the second population of T cells begins 11 days after beginning the culturing of the T cells from a biological sample from a subject in a first cell culture medium. In some embodiments, enriching CD137 (4-1BB)-expressing T cells and/or CD69 expressing T cells from the second population of T cells begins 12 days after beginning the culturing of the T cells from a biological sample from a subject in a first cell culture medium. In some embodiments, enriching CD137 (4-1BB)-expressing T cells and/or CD69 expressing T cells from the second population of T cells begins 13 days after beginning the culturing of the T cells from a biological sample from a subject in a first cell culture medium. In some embodiments, enriching CD137 (4-1BB)-expressing T cells and/or CD69 expressing T cells from the second population of T cells begins 14 days after beginning the culturing of the T cells from a biological sample from a subject in a first cell culture medium. In some embodiments, enriching CD137 (4-1BB)-expressing T cells and/or CD69 expressing T cells from the second population of T cells begins 15 days after beginning the culturing of the T cells from a biological sample from a subject in a first cell culture medium. In some embodiments, enriching CD137 (4-1BB)-expressing T cells and/or CD69 expressing T cells from the second population of T cells begins 16 days after beginning the culturing of the T cells from a biological sample from a subject in a first cell culture medium. In some embodiments, enriching CD137 (4-1BB)-expressing T cells and/or CD69 expressing T cells from the second population of T cells begins 17 days after beginning the culturing of the T cells from a biological sample from a subject in a first cell culture medium. In some embodiments, enriching CD137 (4-1BB)-expressing T cells and/or CD69 expressing T cells from the second population of T cells begins 18 days after beginning the culturing of the T cells from a biological sample from a subject in a first cell culture medium.

In some embodiments, the APCs (i) comprise a polynucleotide sequence encoding the peptide antigen, or (ii) are loaded with the epitope of a peptide antigen. In some embodiments, the peptide antigen is directly added to the first cell culture medium.

In some embodiments, the first cell culture medium comprises a first concentration of the peptide antigen In some embodiments, the method further comprises supplementing the first cell culture medium with an amount of the peptide antigen such that the first cell culture medium comprises a first concentration of the peptide antigen.

In some embodiments, the first concentration of the peptide antigen is from 1 nM to 100 μM. In some embodiments, the first concentration of the peptide antigen is from 100 nM to 10 μM. In some embodiments, the first concentration of the peptide is about 1 μM to about 5 μM.

In some embodiments, the first concentration of the peptide antigen is 1 μM. In some embodiments, the first concentration of the peptide antigen is 2 μM. In some embodiments, the first concentration of the peptide antigen is 3 μM. In some embodiments, the first concentration of the peptide antigen is 4 μM. In some embodiments, the first concentration of the peptide antigen is 5 μM.

In some embodiments, the second cell culture medium comprises a second concentration of the peptide antigen. In some embodiments, the method further comprises supplementing the second cell culture medium with an amount of the peptide antigen such that the second cell culture medium comprises a second concentration of the peptide antigen. In some embodiments, the first concentration of the peptide antigen is from 1 nM to 100 μM. In some embodiments, the first concentration of the peptide antigen is from 100 nM to 10 μM. In some embodiments, the first concentration of the peptide is about 1 μM to about 5 μM. In some embodiments, the first concentration of the peptide antigen is 1 μM. In some embodiments, the first concentration of the peptide antigen is 2 μM. In some embodiments, the first concentration of the peptide antigen is 3 μM. In some embodiments, the first concentration of the peptide antigen is 4 μM. In some embodiments, the first concentration of the peptide antigen is 5 μM.

In some embodiments, culturing the first population of T cells in the second cell culture medium begins at about 9 days after beginning the culturing of the T cells from a biological sample from a subject in a first cell culture medium. In some embodiments, culturing the first population of T cells in the second cell culture medium begins at about 10 days after beginning the culturing of the T cells from a biological sample from a subject in a first cell culture medium. In some embodiments, culturing the first population of T cells in the second cell culture medium begins at about 11 days after beginning the culturing of the T cells from a biological sample from a subject in a first cell culture medium. In some embodiments, culturing the first population of T cells in the second cell culture medium begins at about 12 days after beginning the culturing of the T cells from a biological sample from a subject in a first cell culture medium. In some embodiments, culturing the first population of T cells in the second cell culture medium begins at about 13 days after beginning the culturing of the T cells from a biological sample from a subject in a first cell culture medium. In some embodiments, culturing the first population of T cells in the second cell culture medium begins at about 14 days after beginning the culturing of the T cells from a biological sample from a subject in a first cell culture medium. In some embodiments, culturing the first population of T cells in the second cell culture medium begins at about 15 days after beginning the culturing of the T cells from a biological sample from a subject in a first cell culture medium. In some embodiments, culturing the first population of T cells in the second cell culture medium begins at about 16 days after beginning the culturing of the T cells from a biological sample from a subject in a first cell culture medium. In some embodiments, culturing the first population of T cells in the second cell culture medium begins at about 17 days after beginning the culturing of the T cells from a biological sample from a subject in a first cell culture medium.

In some embodiments, the third cell culture medium comprises a third concentration of the peptide antigen. In some embodiments, the method further comprises supplementing the third cell culture medium with an amount of the peptide antigen such that the third cell culture medium comprises a third concentration of the peptide antigen.

In some embodiments, the third concentration of the peptide antigen is at least 2-fold lower than the first concentration of the peptide antigen.

In some embodiments, the third concentration of the peptide antigen is at least 2-fold lower than the second concentration of the peptide antigen.

In some embodiments, the third concentration of the peptide antigen is at least 3-fold lower than the first concentration of the peptide antigen. In some embodiments, the third concentration of the peptide antigen is at least 4-fold lower than the first concentration of the peptide antigen. In some embodiments, the third concentration of the peptide antigen is at least 5-fold lower than the first concentration of the peptide antigen. In some embodiments, the third concentration of the peptide antigen is at least 6-fold lower than the first concentration of the peptide antigen. In some embodiments, the third concentration of the peptide antigen is at least 7-fold lower than the first concentration of the peptide antigen. In some embodiments, the third concentration of the peptide antigen is at least 8-fold lower than the first concentration of the peptide antigen. In some embodiments, the third concentration of the peptide antigen is at least 9-fold lower than the first concentration of the peptide antigen. In some embodiments, the third concentration of the peptide antigen is at least 10-fold lower than the first concentration of the peptide antigen.

In some embodiments, the third concentration of the peptide antigen is at least 3, 4, 5, 6, 7, 8, 9 or 10-fold lower than the second concentration of the peptide antigen. In some embodiments, the third concentration of the peptide antigen is at least 3-fold lower than the second concentration of the peptide antigen. In some embodiments, the third concentration of the peptide antigen is at least 4-fold lower than the second concentration of the peptide antigen. In some embodiments, the third concentration of the peptide antigen is at least 5-fold lower than the second concentration of the peptide antigen. In some embodiments, the third concentration of the peptide antigen is at least 6-fold lower than the second concentration of the peptide antigen. In some embodiments, the third concentration of the peptide antigen is at least 7-fold lower than the second concentration of the peptide antigen. In some embodiments, the third concentration of the peptide antigen is at least 8-fold lower than the second concentration of the peptide antigen. In some embodiments, the third concentration of the peptide antigen is at least 9-fold lower than the second concentration of the peptide antigen. In some embodiments, the third concentration of the peptide antigen is at least 10-fold lower than the second concentration of the peptide antigen.

In some embodiments, the third concentration of the peptide antigen is from 0.1 nM to 10 μM.

In some embodiments, the third concentration of the peptide antigen is about 0.1 nM, 0.5, nM, 1 nM, 10 nM, 25 nM, 50 nM, 100 nM, 150 nM, 200 nM, 300 nM, 400 nM, 500 nM, 1 μM or 10 μM.

In some embodiments, expanding the second or third population of T cells in the third cell culture medium begins at 11 days after beginning the culturing of the T cells from a biological sample from a subject in a first cell culture medium. In some embodiments, expanding the second or third population of T cells in the third cell culture medium begins at 12 days after beginning the culturing of the T cells from a biological sample from a subject in a first cell culture medium. In some embodiments, expanding the second or third population of T cells in the third cell culture medium begins at 13 days after beginning the culturing of the T cells from a biological sample from a subject in a first cell culture medium. In some embodiments, expanding the second or third population of T cells in the third cell culture medium begins at 14 days after beginning the culturing of the T cells from a biological sample from a subject in a first cell culture medium. In some embodiments, expanding the second or third population of T cells in the third cell culture medium begins at 15 days after beginning the culturing of the T cells from a biological sample from a subject in a first cell culture medium. In some embodiments, expanding the second or third population of T cells in the third cell culture medium begins at 16 days after beginning the culturing of the T cells from a biological sample from a subject in a first cell culture medium. In some embodiments, expanding the second or third population of T cells in the third cell culture medium begins at 17 days after beginning the culturing of the T cells from a biological sample from a subject in a first cell culture medium.

In some embodiments, expanding the second or third population of T cells in the third cell culture medium begins 1, 2, 3 4 or 5 days after enriching CD137 (4-1BB)-expressing T cells and/or CD69 expressing T cells from the second population of T cells.

In some embodiments, expanding the second or third population of T cells in a third cell culture medium comprises expanding the second or third population of T cells in the presence of an increasing concentration of the peptide antigen.

In some embodiments, expanding the second or third population of T cells in the presence of an increasing concentration of the peptide antigen comprises expanding the second or third population of T cells in a fourth cell culture medium comprising a fourth concentration of the peptide antigen

In some embodiments, expanding the second or third population of T cells in the presence of an increasing concentration of the peptide antigen comprises supplementing the third cell culture medium with an amount of the peptide antigen such that the third cell culture medium comprises a fourth concentration of the peptide antigen, wherein the fourth concentration of the peptide antigen is at least 1.1-fold higher than the third concentration of the peptide antigen. In some embodiments, the fourth concentration of the peptide antigen is at least 3-fold higher than the third concentration of the peptide antigen. In some embodiments, the fourth concentration of the peptide antigen is at least 2-fold higher than the third concentration of the peptide antigen. In some embodiments, the fourth concentration of the peptide antigen is at least 4-fold higher than the third concentration of the peptide antigen. In some embodiments, the fourth concentration of the peptide antigen is at least 5-fold higher than the third concentration of the peptide antigen. In some embodiments, the fourth concentration of the peptide antigen is at least 6-fold higher than the third concentration of the peptide antigen. In some embodiments, the fourth concentration of the peptide antigen is at least 7-fold higher than the third concentration of the peptide antigen. In some embodiments, the fourth concentration of the peptide antigen is at least 8-fold higher than the third concentration of the peptide antigen. In some embodiments, the fourth concentration of the peptide antigen is at least 9-fold higher than the third concentration of the peptide antigen. In some embodiments, the fourth concentration of the peptide antigen is at least 10-fold higher than the third concentration of the peptide antigen.

In some embodiments, the fourth concentration of the peptide antigen is from 1 nM to 50 μM.

In some embodiments, the fourth concentration of the peptide antigen is about 1 nM. In some embodiments, the fourth concentration of the peptide antigen is about 10 nM. In some embodiments, the fourth concentration of the peptide antigen is about 25 nM. In some embodiments, the fourth concentration of the peptide antigen is about 50 nM. In some embodiments, the fourth concentration of the peptide antigen is about 100 nM. In some embodiments, the fourth concentration of the peptide antigen is about 150 nM. In some embodiments, the fourth concentration of the peptide antigen is about 200 nM. In some embodiments, the fourth concentration of the peptide antigen is about 300 nM. In some embodiments, the fourth concentration of the peptide antigen is about 400 nM. In some embodiments, the fourth concentration of the peptide antigen is about 500 nM. In some embodiments, the fourth concentration of the peptide antigen is about 600 nM. In some embodiments, the fourth concentration of the peptide antigen is about 700 nM. In some embodiments, the fourth concentration of the peptide antigen is about 800 nM. In some embodiments, the fourth concentration of the peptide antigen is about 900 nM. In some embodiments, the fourth concentration of the peptide antigen is about 1 μM. In some embodiments, the fourth concentration of the peptide antigen is about 10 μM. In some embodiments, the fourth concentration of the peptide antigen is about 25 μM. In some embodiments, the fourth concentration of the peptide antigen is about 50 μM.

In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 12, 13, 14, 15, 16, 17, 18, 19 or 20 days after beginning the culturing of the T cells from a biological sample from a subject in a first cell culture medium.

In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 1 day after beginning expanding the second or third population of T cells in the third cell culture medium. In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 2 days after beginning expanding the second or third population of T cells in the third cell culture medium. In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 3 days after beginning expanding the second or third population of T cells in the third cell culture medium. In some embodiments, expanding the second or third population of T cells in a fourth cell culture medium comprising a fourth concentration of the peptide antigen or supplementing the third cell culture medium with an amount of the peptide antigen such that the third cell culture medium comprises a fourth concentration of the peptide antigen begins 4 days after beginning expanding the second or third population of T cells in the third cell culture medium.

In some embodiments, expanding the second or third population of T cells in the presence of an increasing concentration of the peptide antigen comprises supplementing the fourth cell culture medium with an amount of the peptide antigen such that the fourth cell culture medium comprises a fifth concentration of the peptide antigen, wherein the fifth concentration of the peptide antigen is at least 1.1-fold higher than the fourth concentration of the peptide antigen.

In some embodiments, the fifth concentration of the peptide antigen is at least 2-fold higher than the third concentration of the peptide antigen and/or the fourth concentration of the peptide antigen. In some embodiments, the fifth concentration of the peptide antigen is at least 3-fold higher than the third concentration of the peptide antigen and/or the fourth concentration of the peptide antigen. In some embodiments, the fifth concentration of the peptide antigen is at least 4-fold higher than the third concentration of the peptide antigen and/or the fourth concentration of the peptide antigen. In some embodiments, the fifth concentration of the peptide antigen is at least 5-fold higher than the third concentration of the peptide antigen and/or the fourth concentration of the peptide antigen. In some embodiments, the fifth concentration of the peptide antigen is at least 6-fold higher than the third concentration of the peptide antigen and/or the fourth concentration of the peptide antigen. In some embodiments, the fifth concentration of the peptide antigen is at least 7-fold higher than the third concentration of the peptide antigen and/or the fourth concentration of the peptide antigen. In some embodiments, the fifth concentration of the peptide antigen is at least 8-fold higher than the third concentration of the peptide antigen and/or the fourth concentration of the peptide antigen. In some embodiments, the fifth concentration of the peptide antigen is at least 9-fold higher than the third concentration of the peptide antigen and/or the fourth concentration of the peptide antigen. In some embodiments, the fifth concentration of the peptide antigen is at least 10-fold higher than the third concentration of the peptide antigen and/or the fourth concentration of the peptide antigen.

In some embodiments, the fifth concentration of the peptide antigen is from 10 nM to 100 μM.

In some embodiments, the fifth concentration of the peptide antigen is about 10 nM. In some embodiments, the fifth concentration of the peptide antigen is about 25 nM. In some embodiments, the fifth concentration of the peptide antigen is 50 nM. In some embodiments, the fifth concentration of the peptide antigen is 100 nM. In some embodiments, the fifth concentration of the peptide antigen is 150 nM. In some embodiments, the fifth concentration of the peptide antigen is 200 nM, In some embodiments, the fifth concentration of the peptide antigen is 300 nM. In some embodiments, the fifth concentration of the peptide antigen is 400 nM. In some embodiments, the fifth concentration of the peptide antigen is 500 nM. In some embodiments, the fifth concentration of the peptide antigen is 600 nM. In some embodiments, the fifth concentration of the peptide antigen is 700 nM. In some embodiments, the fifth concentration of the peptide antigen is 800 nM. In some embodiments, the fifth concentration of the peptide antigen is 900 nM. In some embodiments, the fifth concentration of the peptide antigen is 1 μM. In some embodiments, the fifth concentration of the peptide antigen is 10 μM, In some embodiments, the fifth concentration of the peptide antigen is 25 μM, In some embodiments, the fifth concentration of the peptide antigen is 50 μM. In some embodiments, the fifth concentration of the peptide antigen is 75 μM. In some embodiments, the fifth concentration of the peptide antigen is 100 μM.

In some embodiments, expanding the second or third population of T cells in a fifth cell culture medium comprising a fifth concentration of the peptide antigen or supplementing the fourth cell culture medium with an amount of the peptide antigen such that the fourth cell culture medium comprises a fifth concentration of the peptide antigen. In some embodiments, the expanding the T cells in a fifth concentration of the peptide antigen begins 13 days after beginning the culturing of the T cells from a biological sample from a subject in a first cell culture medium. In some embodiments, the expanding the T cells in a fifth concentration of the peptide antigen begins 14 days after beginning the culturing of the T cells from a biological sample. In some embodiments, the expanding the T cells in a fifth concentration of the peptide antigen begins 15 days after beginning the culturing of the T cells from a biological sample. In some embodiments, the expanding the T cells in a fifth concentration of the peptide antigen begins 16 days after beginning the culturing of the T cells from a biological sample. In some embodiments, the expanding the T cells in a fifth concentration of the peptide antigen begins 17 days after beginning the culturing of the T cells from a biological sample. In some embodiments, the expanding the T cells in a fifth concentration of the peptide antigen begins 18 days after beginning the culturing of the T cells from a biological sample. In some embodiments, the expanding the T cells in a fifth concentration of the peptide antigen begins 19 days after beginning the culturing of the T cells from a biological sample. In some embodiments, the expanding the T cells in a fifth concentration of the peptide antigen begins 20 days after beginning the culturing of the T cells from a biological sample. In some embodiments, the expanding the T cells in a fifth concentration of the peptide antigen begins 21 days after beginning the culturing of the T cells from a biological sample.

In some embodiments, expanding the second or third population of T cells in a fifth cell culture medium comprising a fifth concentration of the peptide antigen begins 1, 2, 3, 4 or 5 days after expanding the second or third population of T cells in the fourth cell culture medium comprising the fourth concentration of the peptide antigen. In some embodiments, supplementing the fourth cell culture medium with an amount of the peptide antigen such that the fourth cell culture medium comprises a fifth concentration of the peptide antigen 1, 2, 3, 4 or 5 days after supplementing the third cell culture medium with an amount of the peptide antigen such that the third cell culture medium comprises a fourth concentration of the peptide antigen.

In some embodiments, expanding the second or third population of T cells in a fifth cell culture medium comprising a fifth concentration of the peptide antigen or supplementing the fourth cell culture medium with an amount of the peptide antigen such that the fourth cell culture medium comprises a fifth concentration of the peptide antigen begins 2, 3, 4, 5 or 6 days after expanding the second or third population of T cells in a third cell culture medium comprising a third concentration of the peptide antigen or 2, 3, 4, 5 or 6 days after supplementing the third cell culture medium with an amount of the peptide antigen such the third cell culture medium comprises a third concentration of the peptide antigen.

In some embodiments, the number of antigen-specific T cells in the second or third population of T cells is greater than the number of antigen-specific T cells in the first population of T cells.

In some embodiments, the frequency of antigen-specific T cells in the second or third population of T cells is greater than the frequency of antigen-specific T cells in the first population of T cells, wherein the frequency of antigen-specific T cells in a population of T cells is the [number of antigen-specific T cells in the population]/[total number of T cells in the population]×100.

In some embodiments, the frequency of antigen-specific T cells in the therapeutic population of T cells is greater than the frequency of antigen-specific T cells in the first population of T cells, wherein the frequency of antigen-specific T cells in a population of T cells is the [number of antigen-specific T cells in the population]/[total number of T cells in the population]×100.

In some embodiments, the frequency of antigen-specific T cells in the therapeutic population of T cells is greater than the frequency of antigen-specific T cells in the second population of T cells, wherein the frequency of antigen-specific T cells in a population of T cells is the [number of antigen-specific T cells in the population]/[total number of T cells in the population]×100.

In some embodiments, the frequency of antigen-specific T cells in the therapeutic population of T cells is greater than the frequency of antigen-specific T cells in the third population of T cells, wherein the frequency of antigen-specific T cells in a population of T cells is the [number of antigen-specific T cells in the population]/[total number of T cells in the population]×100.

In some embodiments, culturing of the first population of T cells is performed for a period of from 5 to 25 days. In some embodiments, culturing of the first population of T cells is performed for a period of from 7 to 16 days. In some embodiments, culturing of the first population of T cells is performed for a period of from 13 to 15 days. In some embodiments, culturing of the first population of T cells is performed for a period of about 13 or 14 days.

In some embodiments, culturing of the second population of T cells is performed for a period of 1 day. In some embodiments, culturing of the second population of T cells is performed for a period of 2 days. In some embodiments, culturing of the second population of T cells is performed for a period of 3 days. In some embodiments, culturing of the second population of T cells is performed for a period of 4 days.

In some embodiments, culturing of the second population of T cells is performed for a period of from 5 to 25 days. In some embodiments, culturing of the second population of T cells is performed for a period of from 7 to 14 days. In some embodiments, culturing of the second population of T cells is performed for a period of from 11 to 13 days. In some embodiments, culturing of the second population of T cells is performed for a period of 21 days or less. In some embodiments, culturing of the second population of T cells is performed for a period of about 12 days.

In some embodiments, expanding the second or third population of T cells is performed for a period of from 5 to 25 days. In some embodiments, expanding the second or third population of T cells is performed for a period of from 7 to 14 days. In some embodiments, expanding the second or third population of T cells is performed for a period of from 11 to 13 days. In some embodiments, expanding the second or third population of T cells is performed for a period of 21 days or less. In some embodiments, expanding the second or third population of T cells is performed for a period of about 12 days.

In some embodiments, expanding the second or third population of T cells is performed for a period of from 4 to 24 days. In some embodiments, expanding the second or third population of T cells is performed for a period of from 6 to 13 days. In some embodiments, expanding the second or third population of T cells is performed for a period of from 10 to 12 days. In some embodiments, expanding the second or third population of T cells is performed for a period of 20 days or less. In some embodiments, expanding the second or third population of T cells is performed for a period of about 11 days.

In some embodiments, the method expands antigen-specific T cells.

In some embodiments, the method expands naive T cells from the first population of T cells. In some embodiments, the method expands naive T cells from the first population of T cells that have become antigen-specific T cells.

In some embodiments, the method comprises expanding antigen-specific T cells. In some embodiments, culturing T cells from a biological sample from a subject in a first cell culture medium expands antigen-specific T cells. In some embodiments, culturing the first population of T cells in a second cell culture medium expands antigen-specific T cells. In some embodiments, expanding the second or third population of T cells in a third cell culture medium expands antigen-specific T cells. In some embodiments, the first population of T cells is not obtained from a tumor infiltrating lymphocyte (TIL) sample.

In some embodiments, the first and second culture mediums are the same.

In some embodiments, the first and second culture mediums are different.

In some embodiments, the first culture medium comprises GM-CSF, IL-4, FLT3L, TNF-α, IL-1β, PGE1, IL-6, IL-7, IL-12, IFN-α, R848, LPS, ss-rna40, poly I:C, or any combination thereof. In some embodiments, the second culture medium comprises a soluble anti-CD3 antibody, an anti-CD3 antibody conjugated to a bead, soluble anti-CD28 antibody, an anti-CD28 antibody conjugated to a bead, insulin, one or more non-essential amino acids, glucose, glutamine, IL-2, IL-7, IL-15, IL-12, a CD137 agonist, an AKT inhibitor, a MEM vitamin solution, sodium pyruvate or any combination thereof. In some embodiments, the first culture medium comprises FMS-like tyrosine kinase 3 receptor ligand (FLT3L). In some embodiments, the second culture medium comprises FLT3L. In some embodiments, the second culture medium does not comprise additional APCs. In some embodiments, a number of APCs present in the second or the third culture medium is less than the number of APCs present in the first cell culture medium. In some embodiments, supplementing does not comprise supplementing with APCs. In some embodiments, the method comprises enriching CD137-expressing T cells from the second population of T cells after (a) and before (b). In some embodiments, enriching comprises enriching with an enriching reagent comprising an anti-CD137 reagent.

In some embodiments, the stimulated T cell is a population of immune cells comprising the activated T cells stimulated with APCs comprising a neoantigenic peptide-MHC complex. In some embodiments, a method can comprise incubating a population of immune cells from a biological sample with APCs comprising a peptide-MHC complex, thereby obtaining a stimulated immune cell sample; determining expression of one or more cell markers of at least one immune cell of the stimulated immune cell sample; and determining binding of the at least one immune cell of the stimulated immune cell sample to a peptide-MHC complex; wherein determining expression of certain cell surface markers or other determinant markers, such as intracellular factors, or released agents, such as cytokines etc., and determining binding to the neoantigen-MHC complex are performed simultaneously. In some embodiments, the one or more cell markers comprise TNF-α, IFN-γ, LAMP-1, CD137, IL-2, IL-17A, Granzyme B, PD-1, CD25, CD69, TIM3, LAG3, CTLA-4, CD62L, CD45RA, CD45RO, FoxP3, or any combination thereof. In some embodiments, the one or more cell markers comprise a cytokine. In some embodiments, the one or more cell markers comprise a degranulation marker. In some embodiments, the one or more cell markers comprise a cell-surface marker. In some embodiments, the one or more cell markers comprise a protein. In some embodiments, determining binding of the at least one immune cell of the stimulated immune cell sample to the peptide-MHC complex comprises determining binding of the at least one immune cell of the stimulated immune cell sample to a MHC tetramer comprising the peptide and the MHC of the peptide-MHC complex. In some embodiments, the MHC is a class I MHC or a class II MHC. In some embodiments, the peptide-MHC complex comprises one or more labels.

In some embodiments, activation of T cell is verified by detecting the release of a cytokine by the activated T cell. In some embodiments, the cytokine is one or more of: TNF-α, IFN-γ, or IL-2. In some embodiments the activation of T cell is verified by its specific antigen binding and cytokine release. In some embodiments, the activation of T cells is verified by its ability to kill tumor cells in vitro. A sample of activated T cells may be used to verify the activation status of the T cells. In some embodiments, a sample from the T cells is withdrawn from the T cell culture to determine the cellular composition and activation state by flow cytometry.

In some embodiments, a percentage of the at least one antigen specific T cell in the composition is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total T cells or total immune cells. In some embodiments, the percentage of the at least one antigen specific T cells in the composition is about 5%. In some embodiments, the percentage of the at least one antigen specific T cells in the composition is about 7%. In some embodiments, the percentage of the at least one antigen specific T cells in the composition is about 10%. In some embodiments, the percentage of the at least one antigen specific T cells in the composition is about 12%. In some embodiments, the percentage of the at least one antigen specific T cells in the composition is about 15%. In some embodiments, the percentage of the at least one antigen specific T cells in the composition is about 20%. In some embodiments, the percentage of the at least one antigen specific T cells in the composition is about 25%. In some embodiments, the percentage of the at least one antigen specific T cells in the composition is about 30%. In some embodiments, the percentage of the at least one antigen specific T cells in the composition is about 40%. In some embodiments, the percentage of the at least one antigen specific T cells in the composition is about 50%. In some embodiments, the percentage of the at least one antigen specific T cells in the composition is about 60%. In some embodiments, the percentage of the at least one antigen specific T cells in the composition is about 70%. In some embodiments, the percentage of the at least one antigen specific T cells in the composition is about 80%. In some embodiments, the percentage of the at least one antigen specific T cells in the composition is about 90%.

In some embodiments, a percentage of at least one antigen specific CD8+ T cell in the composition is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total CD4+ T cells, total CD8+ T cells, total T cells or total immune cells. In some embodiments, the percentage of the at least one antigen specific CD8+ T cells in the composition is about 5%. In some embodiments, the percentage of the at least one antigen specific CD8+ T cells in the composition is about 7%. In some embodiments, the percentage of the at least one antigen specific CD8+ T cells in the composition is about 10%. In some embodiments, the percentage of the at least one antigen specific CD8+ T cells in the composition is about 12%. In some embodiments, the percentage of the at least one antigen specific CD8+ T cells in the composition is about 15%. In some embodiments, the percentage of the at least one antigen specific CD8+ T cells in the composition is about 20%. In some embodiments, the percentage of the at least one antigen specific CD8+ T cells in the composition is about 25%. In some embodiments, the percentage of the at least one antigen specific CD8+ T cells in the composition is about 30%. In some embodiments, the percentage of the at least one antigen specific CD8+ T cells in the composition is about 40%. In some embodiments, the percentage of the at least one antigen specific CD8+ T cells in the composition is about 50%. In some embodiments, the percentage of the at least one antigen specific CD8+ T cells in the composition is about 60%. In some embodiments, the percentage of the at least one antigen specific CD8+ T cells in the composition is about 70% of total CD4+ T cells, total CD8+ T cells, total T cells or total immune cells.

In some embodiments, a percentage of at least one antigen specific CD4+ T cell in the composition is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total CD4+ T cells, total CD8+ T cells, total T cells or total immune cells.

In some embodiments, a percentage of the at least one antigen specific T cell in the biological sample is at most about 0.00001%, 0.00005%, 0.0001%, 0.0005%, 0.001%, 0.005%, 0.01%, 0.05%, 0.1% or 0.5% of total CD4+ T cells, total CD8+ T cells, total T cells or total immune cells.

In some embodiments, a percentage of at least one antigen specific CD8+ T cell in the biological sample is at most about 0.00001%, 0.00005%, 0.0001%, 0.0005%, 0.001%, 0.005%, 0.01%, 0.05%, 0.1% or 0.5% of total CD4+ T cells, total CD8+ T cells, total T cells or total immune cells.

In some embodiments, a percentage of at least one antigen specific CD4+ T cell in the biological sample is at most about 0.00001%, 0.00005%, 0.0001%, 0.0005%, 0.001%, 0.005%, 0.01%, 0.05%, 0.1% or 0.5% of total CD4+ T cells, total CD8+ T cells, total T cells or total immune cells.

In some embodiments, the antigen is a neoantigen, a tumor associated antigen, an overexpressed antigen, a viral antigen, a minor histocompatibility antigen or a combination thereof.

In some embodiments, the number of at least one antigen specific CD8+ T cell in the composition is at least about 1×10\hat{ }6, 2×10\hat{ }6, 5×10\hat{ }6, 1×10\hat{ }7, 2×10\hat{ }7, 5×10\hat{ }7, 1×10\hat{ }8, 2×10\hat{ }8, or 5×10\hat{ }8, antigen specific CD8+ T cells.

In some embodiments, a number of at least one antigen specific CD4+ T cell in the composition is at least about 1×10\hat{ }6, 2×10\hat{ }6, 5×10\hat{ }6, 1×10\hat{ }7, 2×10″7, 5×10\hat{ }7, 1×10\hat{ }8, 2×10\hat{ }8, or 5×10\hat{ }8, antigen specific CD4+ T cells.

### Pharmaceutical Compositions

Provided herein are compositions (e.g., pharmaceutical compositions) comprising a population of immune cells. The compositions can comprise at least one antigen specific T cells comprising a T cell receptor (TCR). The compositions can comprise at least one antigen specific T cells comprising a T cell receptor (TCR) specific to at least one peptide antigen sequence.

Pharmaceutical compositions can be formulated using one or more physiologically acceptable carriers including excipients and auxiliaries which facilitate processing of the active agents into preparations which can be used pharmaceutically. Proper formulation can be dependent upon the route of administration chosen. Any of the well-known techniques, carriers, and excipients can be used as suitable and as understood in the art. In some cases, a pharmaceutical composition is formulated as cell based therapeutic, e.g., a T cell therapeutic. In some embodiments, the pharmaceutical composition comprises a peptide-based therapy, a nucleic acid-based therapy, an antibody based therapy, and/or a cell based therapy. In some embodiments, a pharmaceutical composition comprises a peptide-based therapeutic, or nucleic acid based therapeutic in which the nucleic acid encodes the polypeptides. In some embodiments, a pharmaceutical composition comprises a peptide-based therapeutic, or nucleic acid based therapeutic in which the nucleic acid encodes the polypeptides; wherein the peptide-based therapeutic, or nucleic acid based therapeutic are comprised in a cell, wherein the cell is a T cell. In some embodiments, a pharmaceutical composition comprises as an antibody based therapeutic. A composition can comprise T cells specific for two or more immunogenic antigen or neoantigen peptides.

In one aspect, provided herein is a pharmaceutical composition comprising (a) a population of immune cells comprising T cells from a biological sample, wherein the T cells comprise at least one antigen specific T cell that is an APC-stimulated T cell and comprises a T cell receptor (TCR) specific to at least one peptide antigen sequence, wherein the APC is a FLT3L-stimulated APC; and (b) a pharmaceutically acceptable excipient.

In one aspect, provided herein is a pharmaceutical composition comprising: (a) a population of immune cells from a biological sample comprising at least one antigen specific T cell comprising a T cell receptor (TCR) specific to at least one peptide antigen sequence, and (b) a pharmaceutically acceptable excipient; wherein an amount of immune cells expressing CD14 and/or CD25 in the population is proportionally different from an amount of immune cells expressing CD14 and/or CD25 in the biological sample. In some embodiments, the at least one antigen specific T cell comprises at least one APC-stimulated T cell. In some embodiments, the amount of immune cells expressing CD14 and/or CD25 in the population is proportionally less than the amount of immune cells expressing CD14 and/or CD25 in the biological sample. In some embodiments, the amount of immune cells expressing CD14 and/or CD25 in the population is proportionally more than the amount of immune cells expressing CD14 and/or CD25 in the biological sample. In some embodiments, the at least one antigen specific T cell comprises at least one CD4+ T cell. In some embodiments, the at least one antigen specific T cell comprises at least one CD8+ T cell. In some embodiments, the at least one antigen specific T cell comprises at least one CD4 enriched T cell. In some embodiments, the at least one antigen specific T cell comprises at least one CD8 enriched T cell. In some embodiments, the at least one antigen specific T cell comprises a memory T cell. In some embodiments, the at least one antigen specific T cell comprises a memory CD4+ T cell. In some embodiments, the at least one antigen specific T cell comprises a memory CD8+ T cell. In some embodiments, a percentage of the at least one antigen specific T cell in the composition is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total T cells or total immune cells. In some embodiments, a percentage of at least one antigen specific CD8+ T cell in the composition is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total CD4+ T cells, total CD8+ T cells, total T cells or total immune cells.

Pharmaceutical compositions can include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Acceptable carriers are physiologically acceptable to the administered patient and retain the therapeutic properties of the compounds with/in which it is administered. Acceptable carriers and their formulations are generally described in, for example, Remington' pharmaceutical Sciences (18^{th} ed. A. Gennaro, Mack Publishing Co., Easton, P A 1990). One example of carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. Nor should an acceptable carrier alter the specific activity of the neoantigens.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes.

In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

When the compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that acts as a stabilizing agent, increases or delays absorption, or increases or delays clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (e.g., Fix (1996) Pharm Res. 13:1760 1764; Samanen (1996) J. Pharm. Pharmacol. 48:119 135; and U.S. Pat. No. 5,391,377).

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations in the blood are contemplated.

In some embodiments, the present invention is directed to an immunogenic composition, e.g., a pharmaceutical composition capable of raising a neoantigen-specific response (e.g., a humoral or cell-mediated immune response). In some embodiments, the immunogenic composition comprises neoantigen therapeutics (e.g., peptides, polynucleotides, TCR, CAR, cells containing TCR or CAR, dendritic cell containing polypeptide, dendritic cell containing polynucleotide, antibody, etc.) described herein corresponding to a tumor specific antigen or neoantigen.

In some embodiments, a pharmaceutical composition described herein is capable of raising a specific cytotoxic T cells response, specific helper T cell response, or a B cell response.

In some embodiments, antigen polypeptides or polynucleotides can be provided as antigen presenting cells (e.g., dendritic cells) containing such polypeptides or polynucleotides. In other embodiments, such antigen presenting cells are used to stimulate T cells for use in patients. In some embodiments, the antigen presenting cells are dendritic cells. In related embodiments, the dendritic cells are autologous dendritic cells that are pulsed with the neoantigen peptide or nucleic acid. The neoantigen peptide can be any suitable peptide that gives rise to an appropriate T cell response. In some embodiments, the T cell is a CTL. In some embodiments, the T cell is a HTL. Thus, one embodiment of the present disclosure is an immunogenic composition containing at least one antigen presenting cell (e.g., a dendritic cell) that is pulsed or loaded with one or more neoantigen polypeptides or polynucleotides described herein. In some embodiments, such APCs are autologous (e.g., autologous dendritic cells). Alternatively, peripheral blood mononuclear cells (PBMCs) isolated from a patient can be loaded with neoantigen peptides or polynucleotides ex vivo. In related embodiments, such APCs or PBMCs are injected back into the patient. The polynucleotide can be any suitable polynucleotide that is capable of transducing the dendritic cell, thus resulting in the presentation of a neoantigen peptide and induction of immunity. In some embodiments, such antigen presenting cells (APCs) (e.g., dendritic cells) or peripheral blood mononuclear cells (PBMCs) are used to stimulate a T cell (e.g., an autologous T cell). In related embodiments, the T cell is a CTL. In other related embodiments, the T cell is an HTL. In some embodiments, the T cells are CD8⁺ T cells. In some embodiments, the T cells are CD4⁺ T cells. Such T cells are then injected into the patient.

In some embodiments, CTL is injected into the patient. In some embodiments, HTL is injected into the patient. In some embodiments, both CTL and HTL are injected into the patient. Administration of either therapeutic can be performed simultaneously or sequentially and in any order.

In some embodiments, a pharmaceutical composition (e.g., immunogenic compositions) described herein for therapeutic treatment can be formulated for parenteral, topical, nasal, oral or local administration. In some embodiments, the pharmaceutical compositions described herein are administered parenterally, e.g., intravenously, subcutaneously, intradermally, or intramuscularly. In some embodiments, the composition can be administered intratumorally. The compositions can be administered at the site of surgical excision to induce a local immune response to the tumor. In some embodiments, described herein are compositions for parenteral administration which comprise a solution of the neoantigen peptides and immunogenic compositions are dissolved or suspended in an acceptable carrier, for example, an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The ability of an adjuvant to increase the immune response to an antigen is typically manifested by a significant increase in immune-mediated reaction, or reduction in disease symptoms. For example, an increase in humoral immunity can be manifested by a significant increase in the titer of antibodies raised to the antigen, and an increase in T cell activity can be manifested in increased cell proliferation, or cellular cytotoxicity, or cytokine secretion. An adjuvant can also alter an immune response, for example, by changing a primarily humoral or T helper 2 response into a primarily cellular, or T helper 1 response.

Suitable adjuvants are known in the art (see, WO 2015/095811) and include, but are not limited to poly(I:C), poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel® vector system, PLG microparticles, resiquimod, SRL172, virosomes and other virus-like particles, YF-17D, VEGF trap, R848, β-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described (Dupuis M, et al., Cell Immunol. 1998; 186(1):18-27; Allison A C; Dev Biol Stand. 1998; 92:3-11) (Mosca et al. Frontiers in Bioscience, 2007; 12:4050-4060) (Gamvrellis et al. Immunol & Cell Biol. 2004; 82: 506-516). Also, cytokines can be used. Several cytokines have been directly linked to influencing dendritic cell migration to lymphoid tissues (e.g., TNF-α), accelerating the maturation of dendritic cells into efficient antigen-presenting cells for T-lymphocytes (e.g., GM-CSF, PGE1, PGE2, IL-1, IL-1β, IL-4, IL-6 and CD40L) (U.S. Pat. No. 5,849,589 incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a therapeutic setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell immunogenic pharmaceutical compositions, autologous cellular immunogenic pharmaceutical compositions and polysaccharide conjugates in both prophylactic and therapeutic immunogenic pharmaceutical compositions. Importantly, it enhances dendritic cell maturation and differentiation, resulting in enhanced activation of TH1 cells and strong cytotoxic T-lymphocyte (CTL) generation, even in the absence of CD4⁺ T cell help. The TH1 bias induced by TLR9 stimulation is maintained even in the presence of adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially useful for inducing a strong response when the antigen is relatively weak. They can also accelerate the immune response and enabled the antigen doses to be reduced with comparable antibody responses to the full-dose immunogenic pharmaceutical composition without CpG in some experiments (Arthur M. Krieg, Nature Reviews, Drug Discovery, 5, June 2006, 471-484). U.S. Pat. No. 6,406,705 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, DE), which is a component of the pharmaceutical composition described herein. Other TLR binding molecules such as RNA binding TLR7, TLR8 and/or TLR9 can also be used.

Other examples of useful adjuvants include, but are not limited to, chemically modified CpGs (e.g. CpR, Idera), Poly(I and/or poly C)(e.g., polyI:CI2U), non-CpG bacterial DNA or RNA, ssRNA40 for TLR8, as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which can act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

In some embodiments, an immunogenic composition according to the present disclosure can comprise more than one different adjuvant. Furthermore, the invention encompasses a pharmaceutical composition comprising any adjuvant substance including any of the above or combinations thereof. In some embodiments, the immunogenic composition comprises neoantigen therapeutics (e.g., peptides, polynucleotides, TCR, CAR, cells containing TCR or CAR, dendritic cell containing polypeptide, dendritic cell containing polynucleotide, antibody, etc.) and the adjuvant can be administered separately in any appropriate sequence.

Lipidation can be classified into several different types, such as N-myristoylation, palmitoylation, GPI-anchor addition, prenylation, and several additional types of modifications. N-myristoylation is the covalent attachment of myristate, a C14 saturated acid, to a glycine residue. Palmitoylation is thioester linkage of long-chain fatty acids (C16) to cysteine residues. GPI-anchor addition is glycosyl-phosphatidylinositol (GPI) linkage via amide bond. Prenylation is the thioether linkage of an isoprenoid lipid (e.g. farnesyl (C-15), geranylgeranyl (C-20)) to cysteine residues. Additional types of modifications can include attachment of S-diacylglycerol by a sulfur atom of cysteines, 0-octanoyl conjugation via serine or threonine residues, S-archaeol conjugation to cysteine residues, and cholesterol attachment.

Fatty acids for generating lipidated peptides can include C2 to C30 saturated, monounsaturated, or polyunsaturated fatty acyl groups. Exemplary fatty acids can include palmitoyl, myristoyl, stearoyl and decanoyl groups. In some instances, a lipid moiety that has adjuvant property is attached to a polypeptide of interest to elicit or enhance immunogenicity in the absence of an extrinsic adjuvant. A lipidated peptide or lipopeptide can be referred to as a self-adjuvant lipopeptide. Any of the fatty acids described above and elsewhere herein can elicit or enhance immunogenicity of a polypeptide of interest. A fatty acid that can elicit or enhance immunogenicity can include palmitoyl, myristoyl, stearoyl, lauroyl, octanoyl, and decanoyl groups.

Polypeptides such as naked peptides or lipidated peptides can be incorporated into a liposome. Sometimes, lipidated peptides can be incorporated into a liposome. For example, the lipid portion of the lipidated peptide can spontaneously integrate into the lipid bilayer of a liposome. Thus, a lipopeptide can be presented on the “surface” of a liposome. Exemplary liposomes suitable for incorporation in the formulations include, and are not limited to, multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

Depending on the method of preparation, liposomes can be unilamellar or multilamellar, and can vary in size with diameters ranging from about 0.02 μM to greater than about 10 μm. Liposomes can adsorb many types of cells and then release an incorporated agent (e.g., a peptide described herein). In some cases, the liposomes fuse with the target cell, whereby the contents of the liposome then empty into the target cell. A liposome can be endocytosed by cells that are phagocytic. Endocytosis can be followed by intralysosomal degradation of liposomal lipids and release of the encapsulated agents.

The liposomes provided herein can also comprise carrier lipids. In some embodiments the carrier lipids are phospholipids. Carrier lipids capable of forming liposomes include, but are not limited to dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS). Other suitable phospholipids further include distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or combinations thereof. In some embodiments, the liposomes further comprise a sterol (e.g., cholesterol) which modulates liposome formation. The carrier lipids can be any known non-phosphate polar lipids.

A pharmaceutical composition can be encapsulated within liposomes using well-known technology. Biodegradable microspheres can also be employed as carriers for the pharmaceutical compositions of this invention.

The pharmaceutical composition can be administered in liposomes or microspheres (or microparticles). Methods for preparing liposomes and microspheres for administration to a patient are well known to those of skill in the art. Essentially, material is dissolved in an aqueous solution, the appropriate phospholipids and lipids added, along with surfactants if required, and the material dialyzed or sonicated, as necessary.

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months.

Cell-based immunogenic pharmaceutical compositions can also be administered to a subject. For example, an antigen presenting cell (APC) based immunogenic pharmaceutical composition can be formulated using any of the well-known techniques, carriers, and excipients as suitable and as understood in the art. APCs include monocytes, monocyte-derived cells, macrophages, and dendritic cells. Sometimes, an APC based immunogenic pharmaceutical composition can be a dendritic cell-based immunogenic pharmaceutical composition.

A dendritic cell-based immunogenic pharmaceutical composition can be prepared by any methods well known in the art. In some cases, dendritic cell-based immunogenic pharmaceutical compositions can be prepared through an ex vivo or in vivo method. The ex vivo method can comprise the use of autologous DCs pulsed ex vivo with the polypeptides described herein, to activate or load the DCs prior to administration into the patient. The in vivo method can comprise targeting specific DC receptors using antibodies coupled with the polypeptides described herein. The DC-based immunogenic pharmaceutical composition can further comprise DC activators such as TLR3, TLR-7-8, and CD40 agonists. The DC-based immunogenic pharmaceutical composition can further comprise adjuvants, and a pharmaceutically acceptable carrier.

An adjuvant can be used to enhance the immune response (humoral and/or cellular) elicited in a patient receiving the immunogenic pharmaceutical composition. Sometimes, adjuvants can elicit a Th1-type response. Other times, adjuvants can elicit a Th2-type response. A Th1-type response can be characterized by the production of cytokines such as IFN-γ as opposed to a Th2-type response which can be characterized by the production of cytokines such as IL-4, IL-5 and IL-10.

In some aspects, lipid-based adjuvants, such as MPLA and MDP, can be used with the immunogenic pharmaceutical compositions disclosed herein. Monophosphoryl lipid A (MPLA), for example, is an adjuvant that causes increased presentation of liposomal antigen to specific T Lymphocytes. In addition, a muramyl dipeptide (MDP) can also be used as a suitable adjuvant in conjunction with the immunogenic pharmaceutical formulations described herein.

Adjuvant can also comprise stimulatory molecules such as cytokines. Non-limiting examples of cytokines include: CCL20, α-interferon (IFNα), β-interferon (IFNβ), γ-interferon (IFNγ), platelet derived growth factor (PDGF), TNFα, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-la, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-I, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include: MCP-1, MIP-la, MIP-lp, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-1, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll like receptor. Examples of modulators of toll-like receptors include TLR9 agonists and are not limited to small molecule modulators of toll-like receptors such as Imiquimod. Sometimes, an adjuvant is selected from bacteria toxoids, polyoxypropylene-polyoxyethylene block polymers, aluminum salts, liposomes, CpG polymers, oil-in-water emulsions, or a combination thereof. Sometimes, an adjuvant is an oil-in-water emulsion. The oil-in-water emulsion can include at least one oil and at least one surfactant, with the oil(s) and surfactant(s) being biodegradable (metabolizable) and biocompatible. The oil droplets in the emulsion can be less than 5 μm in diameter, and can even have a sub-micron diameter, with these small sizes being achieved with a microfluidiser to provide stable emulsions. Droplets with a size less than 220 nm can be subjected to filter sterilization.

In some instances, an immunogenic pharmaceutical composition can include carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In another instances, the pharmaceutical preparation is substantially free of preservatives. In other instances, the pharmaceutical preparation can contain at least one preservative. It will be recognized that, while any suitable carrier known to those of ordinary skill in the art can be employed to administer the pharmaceutical compositions described herein, the type of carrier will vary depending on the mode of administration.

An immunogenic pharmaceutical composition can include preservatives such as thiomersal or 2-phenoxyethanol. In some instances, the immunogenic pharmaceutical composition is substantially free from (e.g., <10 μg/mL) mercurial material e.g. thiomersal-free. α-Tocopherol succinate may be used as an alternative to mercurial compounds.

For controlling the tonicity, a physiological salt such as sodium salt can be included in the immunogenic pharmaceutical composition. Other salts can include potassium chloride, potassium dihydrogen phosphate, disodium phosphate, and/or magnesium chloride, or the like.

An immunogenic pharmaceutical composition can have an osmolality of between 200 mOsm/kg and 400 mOsm/kg, between 240-360 mOsm/kg, or within the range of 290-310 mOsm/kg.

An immunogenic pharmaceutical composition can comprise one or more buffers, such as a Tris buffer; a borate buffer; a succinate buffer; a histidine buffer (particularly with an aluminum hydroxide adjuvant); or a citrate buffer. Buffers, in some cases, are included in the 5-20 or 10-50 mM range.

The pH of the immunogenic pharmaceutical composition can be between about 5.0 and about 8.5, between about 6.0 and about 8.0, between about 6.5 and about 7.5, or between about 7.0 and about 7.8.

An immunogenic pharmaceutical composition can be sterile. The immunogenic pharmaceutical composition can be non-pyrogenic e.g. containing <1 EU (endotoxin unit, a standard measure) per dose, and can be <0.1 EU per dose. The composition can be gluten free.

An immunogenic pharmaceutical composition can include detergent e.g. a polyoxyethylene sorbitan ester surfactant (known as ‘Tweens’), or an octoxynol (such as octoxynol-9 (Triton X-100) or t-octylphenoxypolyethoxyethanol). The detergent can be present only at trace amounts. The immunogenic pharmaceutical composition can include less than 1 mg/mL of each of octoxynol-10 and polysorbate 80. Other residual components in trace amounts can be antibiotics (e.g. neomycin, kanamycin, polymyxin B).

An immunogenic pharmaceutical composition can be formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration.

Pharmaceutical compositions comprising, for example, an active agent such as immune cells disclosed herein, in combination with one or more adjuvants can be formulated to comprise certain molar ratios. For example, molar ratios of about 99:1 to about 1:99 of an active agent such as an immune cell described herein, in combination with one or more adjuvants can be used. In some instances, the range of molar ratios of an active agent such as an immune cell described herein, in combination with one or more adjuvants can be selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of an active agent such as an immune cell described herein, in combination with one or more adjuvants can be about 1:9, and in some cases can be about 1:1. The active agent such as an immune cell described herein, in combination with one or more adjuvants can be formulated together, in the same dosage unit e.g., in one vial, suppository, tablet, capsule, an aerosol spray; or each agent, form, and/or compound can be formulated in separate units, e.g., two vials, suppositories, tablets, two capsules, a tablet and a vial, an aerosol spray, and the like.

In some instances, an immunogenic pharmaceutical composition can be administered with an additional agent. The choice of the additional agent can depend, at least in part, on the condition being treated. The additional agent can include, for example, a checkpoint inhibitor agent such as an anti-PD1, anti-CTLA4, anti-PD-L1, anti CD40, or anti-TIM3 agent (e.g., an anti-PD1, anti-CTLA4, anti-PD-L1, anti CD40, or anti-TIM3 antibody); or any agents having a therapeutic effect for a pathogen infection (e.g. viral infection), including, e.g., drugs used to treat inflammatory conditions such as an NSAID, e.g., ibuprofen, naproxen, acetaminophen, ketoprofen, or aspirin. For example, the checkpoint inhibitor can be a PD-1/PD-L1 antagonist selected from the group consisting of: nivolumab (ONO-4538/BMS-936558, MDX1 106, OPDIVO), pembrolizumab (MK-3475, KEYTRUDA), pidilizumab (CT-011), and MPDL328OA (ROCHE). As another example, formulations can additionally contain one or more supplements, such as vitamin C, E or other anti-oxidants.

A pharmaceutical composition comprising an active agent such as an immune cell described herein, in combination with one or more adjuvants can be formulated in conventional manner using one or more physiologically acceptable carriers, comprising excipients, diluents, and/or auxiliaries, e.g., which facilitate processing of the active agents into preparations that can be administered. Proper formulation can depend at least in part upon the route of administration chosen. The agent(s) described herein can be delivered to a patient using a number of routes or modes of administration, including oral, buccal, topical, rectal, transdermal, transmucosal, subcutaneous, intravenous, and intramuscular applications, as well as by inhalation.

The active agents can be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol.

In some embodiments, the pharmaceutical composition comprises a preservative or stabilizer. In some embodiments the preservative or stabilizer is selected from a cytokine, a growth factor or an adjuvant or a chemical substance. In some embodiments, the composition comprises at least one agent that helps preserve cell viability through at least one cycle of freeze-thaw. In some embodiments, the composition comprises at least one agent that helps preserve cell viability through at least more than one cycle of freeze-thaw.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible, biodegradable, such as poly(lactic-co-glycolic)acid. These materials can be made into micro or nanospheres, loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In some instances, pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

### Method of Manufacturing:

Provided herein are methods for antigen specific T cell manufacturing. Provided herein are methods of preparing T cell compositions, such as therapeutic T cell compositions. For example, a method can comprise expanding or inducing antigen specific T cells. Preparing (e.g., inducing or expanding) T cells can also refer to manufacturing T cells, and broadly encompasses procedures to isolate, stimulate, culture, induce, and/or expand any type of T cells (e.g., CD4⁺ T cells and CD8⁺ T cells). In one aspect, provided herein is a method of preparing at least one antigen specific T cell comprising a T cell receptor (TCR) specific to at least one peptide antigen sequence, the method comprising incubating an APC with a population of immune cells from a biological sample depleted of cells expressing CD14 and/or CD25. In some embodiments, the method comprises preparing at least one antigen specific T cell comprising a T cell receptor (TCR) specific to at least one peptide antigen sequence, the method comprising incubating an APC with a population of immune cells from a biological sample depleted of cells expressing CD11b and/or CD19. In some embodiments, the method comprises incubating an APC with a population of immune cells from a biological sample depleted of cells expressing any CD11b and/or CD19 and/or CD14 and/or CD25 or any combination thereof.

In a second aspect, provided here is a method of preparing at least one antigen specific T cell comprising a T cell receptor (TCR) specific to at least one peptide antigen sequence, the method comprising incubating a FMS-like tyrosine kinase 3 receptor ligand (FLT3L)-stimulated APC with a population of immune cells from a biological sample.

In a third aspect, provided herein is a method of preparing a pharmaceutical composition comprising at least one antigen specific T cell comprising a T cell receptor (TCR) specific to at least one peptide antigen sequence, the method comprising: incubating FMS-like tyrosine kinase 3 receptor ligand (FLT3L) with a population of immune cells from a biological sample for a first time period; and thereafter incubating at least one T cell of the biological sample with an APC.

In a fourth aspect, provided herein is a method of preparing at least one antigen specific T cell comprising a T cell receptor (TCR) specific to at least one peptide antigen sequence, the method comprising incubating a population of immune cells from a biological sample with one or more APC preparations for one or more separate time periods of less than 28 days from incubating the population of immune cells with a first APC preparation of the one or more APC preparations, wherein at least one antigen specific memory T cell is expanded, or at least one antigen specific naïve T cell is induced.

In a fifth aspect, provided herein is a method of preparing at least one antigen specific T cell comprising a T cell receptor (TCR) specific to at least one peptide antigen sequence, the method comprising incubating a population of immune cells from a biological sample with 3 or less APC preparations for 3 or less separate time periods, wherein at least one antigen specific memory T cell is expanded or at least one antigen specific naïve T cell is induced.

In some embodiments, a method of preparing antigen specific T cells comprises a T cell receptor (TCR) specific to at least one peptide antigen sequence comprises incubating a population of immune cells from a biological sample with one or more APC preparations for one or more separate time periods, thereby stimulating T cells to become antigen specific T cells, wherein a percentage of antigen specific T cells is at least about 0.00001%, 0.00002%, 0.00005%, 0.0001%, 0.0005%, 0.001%, 0.005%, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total CD4⁺ T cells, total CD8⁺ T cells, total T cells or total immune cells. In some embodiments, a method of preparing antigen specific T cells comprises a T cell receptor (TCR) specific to at least one peptide antigen sequence comprises incubating a population of immune cells from a biological sample with 3 or less APC preparations for 3 or less separate time periods, thereby stimulating T cells to become antigen specific T cells. In some embodiments, a method of preparing antigen specific T cells comprises a T cell receptor (TCR) specific to at least one peptide antigen sequence comprises incubating a population of immune cells from a biological sample with 2 or less APC preparations for 2 or less separate time periods, thereby stimulating T cells to become antigen specific T cells.

In some embodiments, provided herein is a method that comprises incubating a population of immune cells from a biological sample with one or more APC preparations for one or more separate time periods, thereby stimulating T cells to become antigen specific T cells, wherein the APC preparation is a PBMC cell population from which cells expressing one or more cell surface markers are depleted prior to antigen loading of the APC population. In some embodiments, CD14+ cells are depleted prior to antigen loading of an APC population. In some embodiments, CD25+ cells are depleted prior to antigen loading of an APC population. In some embodiments, CD11b+ cells are depleted prior to antigen loading of an APC population. In some embodiments, CD19+ cells are depleted prior to antigen loading of an APC population. In some embodiments, CD3+ cells are depleted prior to antigen loading of an APC population. In some embodiments, CD56+ cells are depleted prior to antigen loading of an APC population. In some embodiments, CD25+ cells and CD14+ cells are depleted prior to antigen loading of an APC population. In some embodiments, CD11b+ and CD25+ cells are depleted prior to antigen loading of an APC population. In some embodiments, CD11b+ and CD14+ cells are depleted prior to antigen loading of an APC population. In some embodiments, CD11b+, CD14+ and CD25+ cells are depleted prior to antigen loading of an APC population. In some embodiments, CD11b+, and CD19+ cells are depleted prior to antigen loading of an APC population. In some embodiments, CD11b+, CD19+ and CD25+ cells are depleted prior to antigen loading of an APC population. In some embodiments, CD11b+, CD14+, CD19+ and CD25+ cells are depleted prior to antigen loading of an APC population. In some embodiments, the method comprises adding to any of the depleted APC population described above, an APC enriched cell PBMC-derived population that are depleted of CD3+ cell. In some embodiments, the APC enriched cell PBMC-derived population is depleted of CD3+ and cells depleted of any one or more of CD11b+, CD14+, CD19+, or CD25+.

In some embodiments, a biological sample comprises peripheral blood mononuclear cells (PBMCs). In some embodiments, the method comprises adding to a PBMC sample, a composition comprising one or more antigenic peptides or nucleic acids encoding the same, thereby loading the APCs within the PBMCs with antigens for antigen presentation to T cells in the PBMC.

In some embodiments, a method comprises: (a) obtaining a biological sample from a subject comprising at least one antigen presenting cell (APC); (b) enriching cells expressing CD11c from the biological sample, thereby obtaining a CD11c⁺ cell enriched sample; (c) incubating the CD11c⁺ cell enriched sample with at least one cytokine or growth factor for a first time period; (d) incubating at least one peptide with the CD11c⁺ enriched sample of (c) for a second time period, thereby obtaining an APC peptide loaded sample; (e) incubating the APC peptide loaded sample with one or more cytokines or growth factors for a third time period, thereby obtaining a matured APC sample; (f) incubating APCs of the matured APC sample with a CD11b and/or CD14 and/or CD25 depleted sample comprising PBMCs for a fourth time period; (g) incubating the PBMCs with APCs of a matured APC sample for a fifth time period; (h) incubating the PBMCs with APCs of a matured APC sample for a sixth time period; and (i) administering at least one T cell of the PBMCs to a subject in need thereof.

In some embodiments, a method comprises: (a) obtaining a biological sample from a subject comprising at least one antigen presenting cell (APC); (b) enriching cells expressing CD14 from the biological sample, thereby obtaining a CD14⁺ cell enriched sample; (c) incubating the CD14⁺ cell enriched sample with at least one cytokine or growth factor for a first time period; (d) incubating at least one peptide with the CD14⁺ enriched sample of (c) for a second time period, thereby obtaining an APC peptide loaded sample; (e) incubating the APC peptide loaded sample with one or more cytokines or growth factors for a third time period, thereby obtaining a matured APC sample; (f) incubating APCs of the matured APC sample with a CD14 and/or CD25 depleted sample comprising PBMCs for a fourth time period; (g) incubating the PBMCs with APCs of a matured APC sample for a fifth time period; (h) incubating the PBMCs with APCs of a matured APC sample for a sixth time period; and (i) administering at least one T cell of the PBMCs to a subject in need thereof.

In some embodiments, a method comprises: (a) obtaining a biological sample from a subject comprising at least one APC and at least one PBMC; (b) depleting cells expressing CD11b and/or CD19 from the biological sample, thereby obtaining a CD11b and/or CD19 cell depleted sample; (c) incubating the CD11b and/or CD19 cell depleted sample with FLT3L for a first time period; (d) incubating at least one peptide with the CD11b and/or CD19 cell depleted sample of (c) for a second time period, thereby obtaining an APC peptide loaded sample; (e) incubating the APC peptide loaded sample with the at least one PBMC for a third time period, thereby obtaining a first stimulated PBMC sample; (f) incubating a PBMC of the first stimulated PBMC sample with an APC of a matured APC sample for a fourth time period, thereby obtaining a second stimulated PBMC sample; (g) incubating a PBMC of the second stimulated PBMC sample with an APC of a matured APC sample for a fifth time period, thereby obtaining a third stimulated PBMC sample; (h) administering at least one T cell of the third stimulated PBMC sample to a subject in need thereof.

In some embodiments, a method comprises: (a) obtaining a biological sample from a subject comprising at least one APC and at least one PBMC; (b) depleting cells expressing CD11b and/or CD19 and/or CD14 and/or CD25 from the biological sample, thereby obtaining a CD11b and/or CD19 cell depleted sample; (c) incubating the CD11b and/or CD19 and/or CD14 and/or CD25 cell depleted sample with FLT3L for a first time period; (d) incubating at least one peptide with the CD11b and/or CD19 and/or CD14 and/or CD25 cell depleted sample of (c) for a second time period, thereby obtaining an APC peptide loaded sample; (e) incubating the APC peptide loaded sample with the at least one PBMC for a third time period, thereby obtaining a first stimulated PBMC sample; (f) incubating a PBMC of the first stimulated PBMC sample with an APC of a matured APC sample for a fourth time period, thereby obtaining a second stimulated PBMC sample; (g) incubating a PBMC of the second stimulated PBMC sample with an APC of a matured APC sample for a fifth time period, thereby obtaining a third stimulated PBMC sample; (h) administering at least one T cell of the third stimulated PBMC sample to a subject in need thereof.

In some embodiments, a method comprises: (a) obtaining a biological sample from a subject comprising at least one APC and at least one PBMC; (b) depleting cells expressing CD14 and/or CD25 from the biological sample, thereby obtaining a CD14 and/or CD25 cell depleted sample; (c) incubating the CD14 and/or CD25 cell depleted sample with FLT3L for a first time period; (d) incubating at least one peptide with the CD14 and/or CD25 cell depleted sample of (c) for a second time period, thereby obtaining an APC peptide loaded sample; (e) incubating the APC peptide loaded sample with the at least one PBMC for a third time period, thereby obtaining a first stimulated PBMC sample; (f) incubating a PBMC of the first stimulated PBMC sample with an APC of a matured APC sample for a fourth time period, thereby obtaining a second stimulated PBMC sample; (g) incubating a PBMC of the second stimulated PBMC sample with an APC of a matured APC sample for a fifth time period, thereby obtaining a third stimulated PBMC sample; (h) administering at least one T cell of the third stimulated PBMC sample to a subject in need thereof.

In some embodiments, a method of preparing at least one antigen specific T cell comprising a T cell receptor (TCR) specific to at least one peptide antigen sequence comprises incubating an APC with a population of immune cells from a biological sample depleted of cells expressing CD14 and/or CD25.

In some embodiments, a method of preparing at least one antigen specific T cell comprising a T cell receptor (TCR) specific to at least one peptide antigen sequence comprises incubating an APC with a population of immune cells from a biological sample depleted of cells expressing CD14, CD25 and/or CD56.

In some embodiments, provided herein is a method of preparing at least one antigen specific T cell comprising a T cell receptor (TCR) specific to at least one peptide antigen sequence, the method comprising incubating a population of immune cells from a biological sample with one or more APC preparations for one or more separate time periods of less than 28 days from incubating the population of immune cells with a first APC preparation of the one or more APC preparations, wherein at least one antigen specific memory T cell is expanded, or at least one antigen specific naïve T cell is induced. In some embodiments, provided herein is a method of preparing at least one antigen specific T cell comprising a T cell receptor (TCR) specific to at least one peptide antigen sequence, the method comprising incubating a population of immune cells from a biological sample with 3 or less APC preparations for 3 or less separate time periods, wherein at least one antigen specific memory T cell is expanded or at least one antigen specific naïve T cell is induced.

In some embodiments, a method of preparing antigen specific T cells comprises a T cell receptor (TCR) specific to at least one peptide antigen sequence comprises contacting a population of immune cells (e.g., PBMCs) to APCs. In some embodiments, a method of preparing antigen specific T cells comprises a T cell receptor (TCR) specific to at least one peptide antigen sequence comprises incubating a population of immune cells (e.g., PBMCs) with APCs for a time period. In some embodiments, the population of immune cells is from a biological sample. In some embodiments, the population of immune cells is from a sample (e.g., a biological sample) depleted of CD14 expressing cells. In some embodiments, the population of immune cells is from a sample (e.g., a biological sample) depleted of CD25 expressing cells. In some embodiments, the population of immune cells is from a sample (e.g., a biological sample) depleted of CD14 expressing cells and CD25 expressing cells.

In some embodiments, a method of preparing at least one antigen specific T cell comprising a T cell receptor (TCR) specific to at least one peptide antigen sequence comprises incubating a FMS-like tyrosine kinase 3 receptor ligand (FLT3L)-stimulated APC with a population of immune cells from a biological sample. In some embodiments, provided herein is a method of preparing a pharmaceutical composition comprising at least one antigen specific T cell comprising a T cell receptor (TCR) specific to at least one peptide antigen sequence, the method comprising: incubating FMS-like tyrosine kinase 3 receptor ligand (FLT3L) with a population of immune cells from a biological sample for a first time period; and thereafter incubating at least one T cell of the biological sample with an APC.

In some embodiments, a method of preparing at least one antigen specific T cell comprising a T cell receptor (TCR) specific to at least one peptide antigen sequence comprises contacting a population of immune cells from a sample (e.g., a biological sample) with FMS-like tyrosine kinase 3 receptor ligand (FLT3L). In some embodiments, a method of preparing at least one antigen specific T cells comprises a T cell receptor (TCR) specific to at least one peptide antigen sequence comprises contacting a population of immune cells from a sample (e.g., a biological sample) with FMS-like tyrosine kinase 3 receptor ligand (FLT3L)-stimulated APCs. In some embodiments, a method of preparing at least one antigen specific T cells comprises a T cell receptor (TCR) specific to at least one peptide antigen sequence comprises incubating a population of immune cells from a sample (e.g., a biological sample) with FMS-like tyrosine kinase 3 receptor ligand (FLT3L)-stimulated APCs. In some embodiments, a method of preparing a pharmaceutical composition comprising at least one antigen specific T cell comprising a T cell receptor (TCR) specific to at least one peptide antigen sequence comprises incubating FMS-like tyrosine kinase 3 receptor ligand (FLT3L) with a population of immune cells from a biological sample (e.g., for a time period); and then contacting T cells of the biological sample to APCs. In some embodiments, a method of preparing at least one antigen specific T cell comprising a T cell receptor (TCR) specific to at least one peptide antigen sequence comprises contacting a population of immune cells from a sample (e.g., a biological sample) to one or more APC preparations. In some embodiments, a method of preparing at least one antigen specific T cell comprising a T cell receptor (TCR) specific to at least one peptide antigen sequence comprises incubating a population of immune cells from a sample (e.g., a biological sample), for example, a PBMC sample to FLT3L for a time period. In some embodiments, the APC comprises APCs in the PBMC sample. In some embodiments, the APCs are separately prepared from a subject's cells from a biological sample to add to the immune cells from a biological sample comprising a T cell. In some embodiments, a method of preparing at least one antigen specific T cell comprising a T cell receptor (TCR) specific to at least one peptide antigen sequence comprises incubating a population of immune cells from a sample (e.g., a biological sample) to one or more APC preparations for one or more separate time periods. In some embodiments, a method of preparing at least one antigen specific T cell comprising a T cell receptor (TCR) specific to at least one peptide antigen sequence comprises incubating a population of immune cells from a sample (e.g., a biological sample) to one or more APC preparations for 1, 2, 3, 4, 5, 6, 7, 8, 9 or 10 separate time periods. In some embodiments, the one or more separate time periods is less than 28 days calculated from incubating the population of immune cells with a first APC preparation of the one or more APC preparations.

In some embodiments, a method of preparing antigen specific T cells comprises a T cell receptor (TCR) specific to at least one peptide antigen sequence comprises incubating a population of immune cells to APCs for a time period, wherein the population of immune cells is from a biological sample comprising PBMCs. In some embodiments, a method of preparing antigen specific T cells comprises a T cell receptor (TCR) specific to at least one peptide antigen sequence comprises incubating a population of immune cells to APCs for a time period, wherein the population of immune cells is from a biological sample depleted of CD14 and/or CD25 expressing cells.

In some embodiments, a method of preparing antigen specific T cells comprising a T cell receptor (TCR) specific to at least one peptide antigen sequence comprises incubating a population of immune cells from a biological sample with FMS-like tyrosine kinase 3 receptor ligand (FLT3L)-stimulated APCs for a time period.

In some embodiments, a method of preparing a pharmaceutical composition comprising antigen specific T cells comprising a T cell receptor (TCR) specific to at least one peptide antigen sequence comprises incubating FMS-like tyrosine kinase 3 receptor ligand (FLT3L) with a population of immune cells from a biological sample; and then contacting T cells of the biological sample with APCs.

In some embodiments, a method of preparing antigen specific T cells comprising a T cell receptor (TCR) specific to at least one peptide antigen sequence comprises incubating a population of immune cells from a biological sample with one or more APC preparations for one or more separate time periods, thereby inducing or expanding antigen specific T cells, wherein the one or more separate time periods is less than 28 days calculated from incubating the population of immune cells with a first APC preparation of the one or more APC preparations. In some embodiments, incubating a population of immune cells from a biological sample with one or more APC preparations for one or more separate time periods is performed in a medium containing IL-7, IL-15, or a combination thereof. In some embodiments, the medium further comprises an indoleamine 2,3-dioxygenase-1 (IDO) inhibitor, an anti-PD-1 antibody, IL-12, or a combination thereof. The IDO inhibitor can be epacadostat, navoximod, 1-Methyltryptophan, or a combination thereof. In some embodiments, the IDO inhibitor may increase the number of antigen-specific CD8⁺ cells. In some embodiments, the IDO inhibitor may maintain the functional profile of memory CD8⁺ T cell responses. The PD-1 antibody may increase the absolute number of antigen-specific memory CD8+ T cell responses. The PD-1 antibody may increase proliferation rate of the cells treated with such antibody. The additional of IL-12 can result in an increase of antigen-specific cells and/or an increase in the frequency of CD8⁺ T cells.

In some embodiments, a method of preparing antigen specific T cells comprising a T cell receptor (TCR) specific to at least one peptide antigen sequence comprises incubating a population of immune cells comprising from a biological sample with one or more APC preparations for one or more separate time periods, thereby expanding or inducing antigen specific T cells, wherein a percentage of antigen specific T cells, antigen specific CD4⁺ T cells, or antigen specific CD8⁺ T cells is at least about 0.00001%, 0.00002%, 0.00005%, 0.0001%, 0.0005%, 0.001%, 0.005%, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total T cells, total CD4⁺ T cells, total CD8⁺ T cells, total immune cells, or total cells.

In some embodiments, a method of preparing antigen specific T cells comprises a T cell receptor (TCR) specific to at least one peptide antigen sequence comprises incubating a population of immune cells from a biological sample with 3 or less APC preparations for 3 or less separate time periods, thereby stimulating T cells to become antigen specific T cells.

In some embodiments, the population of immune cells is from a biological sample depleted of CD14 and/or CD25 expressing cells. In some embodiments, the APCs are FMS-like tyrosine kinase 3 receptor ligand (FLT3L)-stimulated APCs. In some embodiments, the APCs comprise one or more APC preparations. In some embodiments, the APC preparations comprise 3 or less APC preparations. In some embodiments, the APC preparations are incubated with the immune cells sequentially within one or more separate time periods.

In some embodiments, the biological sample is from a subject. In some embodiments, the subject is a human. For example, the subject can be a patient or a donor. In some embodiments, the subject has a disease or disorder. In some embodiments, the disease or disorder is cancer. In some embodiments, the antigen specific T cells comprise CD4⁺ and/or CD8⁺ T cells. In some embodiments, the antigen specific T cells comprise CD4 enriched T cells and/or CD8 enriched T cells. For example, a CD4⁺ T cell and/or CD8⁺ T cell can be isolated from, enriched from, or purified from a biological sample from a subject comprising PBMCs. In some embodiments, the antigen specific T cells are naïve CD4⁺ and/or naïve CD8⁺ T cells. In some embodiments, the antigen specific T cells are memory CD4⁺ and/or memory CD8⁺ T cells.

In some embodiments, the at least one peptide antigen sequence comprises a mutation selected from (A) a point mutation and the cancer antigen peptide binds to the HLA protein of the subject with an IC₅₀ less than 500 nM and a greater affinity than a corresponding wild-type peptide, (B) a splice-site mutation, (C) a frameshift mutation, (D) a read-through mutation, (E) a gene-fusion mutation, and combinations thereof. In some embodiments, each of the at least one peptide antigen sequence binds to a protein encoded by an HLA allele expressed by the subject. In some embodiments, each of the at least one peptide antigen sequence comprises a mutation that is not present in non-cancer cells of the subject. In some embodiments, each of the at least one peptide antigen sequences is encoded by an expressed gene of the subject's cancer cells. In some embodiments, one or more of the at least one peptide antigen sequence has a length of from 8-50 naturally occurring amino acids. In some embodiments, the at least one peptide antigen sequence comprises a plurality of peptide antigen sequences. In some embodiments, the plurality of peptide antigen sequences comprises from 2-50, 3-50, 4-50, 5-5-, 6-50, 7-50, 8-50, 9-50, or 10-50 peptide antigen sequences.

In some embodiments, the APCs comprise APCs loaded with one or more antigen peptides comprising one or more of the at least one peptide antigen sequence. In some embodiments, the APCs are autologous APCs or allogenic APCs. In some embodiments, the APCs comprise dendritic cells (DCs).

In some embodiments, a method comprises depleting CD14 and/or CD25 expressing cells from the biological sample. In some embodiments, depleting CD14⁺ cells comprises contacting a CD14 binding agent to the APCs. In some embodiments, the APCs are derived from CD14⁺ monocytes. In some embodiments, the APCs are enriched from the biological sample. For example, an APC can be isolated from, enriched from, or purified from a biological sample from a subject comprising PBMCs.

In some embodiments, the APCs are stimulated with one or more cytokines or growth factors. In some embodiments, the one or more cytokines or growth factors comprise GM-CSF, IL-4, FLT3L, or a combination thereof. In some embodiments, the one or more cytokines or growth factors comprise IL-4, IFN-γ, LPS, GM-CSF, TNF-α, IL-1β, PGE1, IL-6, IL-7 or a combination thereof.

In some embodiments, the APCs are from a second biological sample. In some embodiments, the second biological sample is from the same subject.

In some embodiments, a percentage of antigen specific T cells generated using the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific T cells in the method is from about 0.1% to about 5%, from about 5% to 10%, from about 10% to 15%, from about 15% to 20%, from about 20% to 25%, from about 25% to 30%, from about 30% to 35%, from about 35% to about 40%, from about 40% to about 45%, from about 45% to about 50%, from about 50% to about 55%, from about 55% to about 60%, from about 60% to 65%, or from about 65% to about 70% of total T cells or total immune cells. In some embodiments, a percentage of antigen specific CD8⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific naïve CD8⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific memory CD8⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific CD4⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific CD4⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific CD8⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific naïve CD8⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific memory CD8⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific CD4⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%.

In some embodiments, a biological sample is freshly obtained from a subject or is a frozen sample.

In some embodiments, a method comprises incubating one or more of the APC preparations with a first medium comprising at least one cytokine or growth factor for a first time period. In some embodiments, the first time period is at lease 1, 2, 3, 4, 5, 6, 7, 8, 9, 10, 11, 12, 13, 14, 15, 16, or 17, or 18 days. In some embodiments, the first time period is no more than 2, 3, 4, 5, 6, 7, 8, 9, 10, 11, 12, 13, 14, 15, 16, 17, or 18 days. In some embodiments, the first time period is at least 1, 2 3, 4, 5, 6, 7, 8, or 9 days. In some embodiments, the first time period is no more than 3, 4, 5, 6, 7, 8, 9, or 10 days. In some embodiments, the at least one cytokine or growth factor comprises GM-CSF, IL-4, FLT3L, TNF-α, IL-1β, PGE1, IL-6, IL-7, IFN-γ, LPS, IFN-α, R848, LPS, ss-rna40, poly I:C, or any combination thereof.

In some embodiments, a method comprises incubating one or more of the APC preparations with at least one peptide for a second time period. In some embodiments, the second time period is no more than 1 hour.

In some embodiments, a method comprises incubating one or more of the APC preparations with a second medium comprising one or more cytokines or growth factors for a third time period, thereby obtaining matured APCs. In some embodiments, the one or more cytokines or growth factors comprises GM-CSF (granulocyte macrophage colony-stimulating factor), IL-4, FLT3L, IFN-γ, LPS, TNF-α, IL-1β, PGE1, IL-6, IL-7, IFN-α, R848 (resiquimod), LPS, ss-rna40, poly I:C, CpG, or a combination thereof. In some embodiments, the third time period is no more than 2, 3, 4, 5, 6, 7, 8, 9, 10, 11, 12, 13, 14, 15, 16, 17, or 18 days. In some embodiments, the third time period is at least 1, 2, 3, 4, 5, 6, 7, 8, 9, 10, 11, 12, 13, 14, 15, 16, or 17 days. In some embodiments, the third time period is no more than 2, 3, 4, or 5 days. In some embodiments, the third time period is at least 1, 2, 3, or 4 days.

In some embodiment, the method further comprises removing the one or more cytokines or growth factors of the second medium after the third time period and before a start of the fourth time period.

### Antigen Loaded PBMCs for T Cell Induction In Vitro

In some embodiments, the methods provided herein comprise isolating PBMCs from a human blood sample, and directly loading the PBMCs with antigens. PBMCs directly contacted with antigens can readily take up antigens by phagocytosis and present antigens to T cells that may be in the culture or added to the culture. In some embodiments, the methods provided herein comprise isolating PBMCs from a human blood sample, and nucleofecting or electroporating a polynucleotide, such as an mRNA, that encodes one or more antigens into the PBMCs. In some embodiments, antigens delivered to PBMCs, instead of antigen presenting cells maturing to DCs, provides a great advantage in terms of time and manufacturing efficiency. The PBMCs may be further depleted of one or more cell types. In some embodiments, the PBMCs may be depleted of CD3+ cells for an initial period of antigen loading and the CD3+ cells returned to the culture for the PBMCs to stimulate the CD3+ T cells. In some embodiments, the PBMCs may be depleted of CD25+ cells. In some embodiments, the PBMCs may be depleted of CD14+ cells. In some embodiments, the PBMCs may be depleted of CD19+ cells. In some embodiments, the PBMCs may be depleted of both CD14 and CD25 expressing cells. In some embodiments, CD11b+ cells are depleted from the PBMC sample before antigen loading. In some embodiments, CD11b+ and CD25+ cells are depleted from the PBMC sample before antigen loading.

In some embodiments, the PBMCs isolated from a human blood sample may be handled as minimally as possible prior to loading with antigens. Increased handling of PBMCs, for example freezing and thawing cells, multiple cell depletion steps, etc., may impair cell health and viability.

In some embodiments, the PBMCs are allogeneic to the subject of therapy. In some embodiments the PBMCs are allogeneic to the subject of adoptive cell therapy with antigen specific T cells.

In some embodiments, the PBMCs are HLA-matched for the subject of therapy. In some embodiments, the PBMCs are allogeneic, and matched for the subject's HLA subtypes, whereas the CD3+ T cells are autologous. The PBMCs are loaded with the respective antigens (e.g. derived from analysis of a peptide presentation analysis platform such as RECON), cocultured with subject's PBMC comprising T cells in order to stimulate antigen specific T cells.

In some embodiments, mRNA is used as the immunogen for uptake and antigen presenting. One advantage of using mRNA over peptide antigens to load PBMCs is that RNA is self adjuvanting, and does not require additional adjuvants. Another advantage of using mRNA is that the peptides are processed and presented endogenously. In some embodiments, the mRNA comprises shortmer constructs, encoding 9-10 amino acid peptides comprising an epitope. In some embodiments, the mRNA comprises longmer constructs, encoding bout 25 amino acid peptides. In some embodiments, the mRNA comprises a concatenation of multiple epitopes. In some embodiments, the concatemers may comprise one or more epitopes from the same antigenic protein. In some embodiments, the concatemers may comprise one or epitopes from several different antigenic proteins. Several embodiments are described in the Examples section. Antigen loading of PBMCs by antigen loading may comprise various mechanisms of delivery ad incorporation of nucleic acid into the PBMCs. In some embodiments, the delivery or mechanism of incorporation includes transfection, electroporation, nucleofection, chemical delivery, for example, lipid encapsulated or liposome mediated delivery.

Use of antigen loaded PBMCs to stimulate T cells saves the maturation time required in a method that generates DCs from a PBMC sample prior to T cell stimulation. In some embodiments, use of antigen loaded PBMCs, for example, mRNA loaded PBMCs as APCs reduces the total manufacturing time by 1, 2, 3, 4, 5, 6, 7, 8, 9, or 10 days. In some embodiments, use of antigen loaded PBMCs as APCs reduces the total manufacturing time by 3 days. In some embodiments, use of antigen loaded PBMCs as APCs reduces the total manufacturing time by 4 days. In some embodiments, use of antigen loaded PBMCs as APCs reduces the total manufacturing time by 5 days. In some embodiments, use of antigen loaded PBMCs as APCs reduces the total manufacturing time by 6 days. In some embodiments, use of antigen loaded PBMCs as APCs reduces the total manufacturing time by 7 days.

In some embodiments, use of mRNA as antigen may be preferred because it is easy to design and manufacture nucleic acids, and transfect the PBMCs. In some embodiments, the use of mRNA comprising the sequence encoding an antigen to express in an APC for antigen presentation may be preferred because the antigen is then endogenously processed and presentation efficiently on the surface pf the APCs. In some embodiments, mRNA loaded PBMCs can stimulate T cells and generate higher antigen specific T cells. In some embodiments, mRNA loaded PBMCs can stimulate T cells and generate higher yield of antigen specific T cells. In some embodiments, mRNA loaded PBMCs can stimulate T cells and generate antigen specific T cells that have higher representation of the input antigens, i.e., reactive to diverse antigens. In some embodiments, mRNA loaded PBMCs can stimulate T cells that have at least 1, 2, 3, 4, 5, 6, 7, 8, 9, 10 or more antigen reactivity in the pool of expanded cells. In some embodiments, the mRNA loaded PBMCs can stimulate T cells that have at least 1, 2, 3, 4, 5, 6, 7, 8, 9, 10 or more antigen reactivity than conventional antigen loaded APCs (such as peptide loaded DCs).

In some embodiments, the PBMC may be directly contacted with an antigen for the antigen presenting cells in the PBMCs to take up and present the antigens to the T cells in the PBMCs. In some embodiments, the PBMCs may be further contacted with APCs loaded with or expressing the antigen, to restimulate the cells with the antigen. In some embodiments, the PBMCs may be further contacted with APCs loaded with or expressing the antigen 1, 2, 3 or more additional times. In some embodiments, the APCs that are used for the restimulation are obtained from the same subject as was previously obtained. Provided herein is a method comprising, in some embodiments, stimulating a population of cells obtained from a subject with an antigen, wherein stimulating comprises culturing T cells from a biological sample (e.g., a PBMC sample, or a leukapheresis sample) in a culture medium comprising antigen presenting cells (APCs) to produce a first population of T cells, e.g. a population of T cells that are responsive to the antigen, and then restimulating the population of T cells with a peptide comprising the antigen for 1, 2, 3, or more times. In some embodiments, the population of T cells that are responsive to the antigen can be enriched before or after or during a restimulation phase, wherein the enrichment is for CD137 (4-1BB)-expressing T cells, thereby obtaining a second or third population or subsequent population of T cells. In some embodiments, the enrichment comprises contacting the population of T cells with an antibody that specifically binds to CD137. In some embodiments, the population of T cells that are responsive to the antigen can be enriched before or after or during a restimulation phase, wherein the enrichment is for CD69-expressing T cells, thereby obtaining a second or third population or subsequent population of T cells. In some embodiments, the enrichment comprises contacting the population of T cells with an antibody that specifically binds to CD69. In some embodiments, the enrichment comprises contacting the population of T cells with an antibody that specifically binds to CD137, or an antibody that specifically binds to CD69, and recovering the antibody-bound cells. In some embodiments, the enrichment comprises contacting the population of T cells with an antibody that specifically binds to CD137, and an antibody that specifically binds to CD69, and recovering the antibody-bound cells. In some embodiments, the restimulating the population of T cells can comprise culturing the T cells with a first concentration of a peptide comprising the antigen for a time period; and a second concentration of the peptide comprising the antigen for a time period, and third concentration of a peptide comprising the antigen for a time period, where in each time period for the stimulation with the first, second or third or subsequent concentration(s) of the peptide comprising the antigen may be the same. In some embodiments, the time periods for the stimulation with the first, second or third or subsequent concentration(s) of the peptide comprising the antigen may be different from each other. In some embodiments, the method comprises culturing T cells from biological sample from a subject in a first cell culture medium comprising antigen presenting cells (APCs) to produce a first population of T cells, wherein the APCs present an epitope of a peptide antigen in complex with an MHC protein, followed by culturing the culturing the first population of T cells in a second cell culture medium to produce a second population of T cells, wherein the second culture medium comprises a first concentration of the peptide comprising the antigen. In some embodiments, the second population of T cells are subjected to enriching CD137 (4-1BB)-expressing T cells to produce the second population of T cells. In some embodiments, the second population of T cells are subjected to enriching CD137 (4-1BB)-expressing T cells to produce a third population of T cells. In some embodiments, the first or the second population of T cells are subjected to enriching CD137 (4-1BB)-expressing T cells. In some embodiments, the first population of T cells are subjected to a stimulation with the peptide comprising the antigen, e.g., the antigen spike. In some embodiments, the antigen spike is administered into the culture medium of the cells comprising the first population of T cells, to generate a second population of T cells.

In some embodiments, the antigen spike is added to the cell culture for stimulating the population of T cells before CD137 enrichment.

In some embodiments, the CD137-expressing enriched cells are further subjected to a stimulation with the peptide comprising the antigen for one, two, three or more periods of time, which in some embodiments may be referred to as antigen pulse phase. In some embodiments, the peptide concentration for the stimulations varies each time. In some embodiments, the CD137-expressing enriched cells are subjected to an increasing concentration of the peptide comprising the antigen. In some embodiments, the CD137-expressing enriched cells are subjected to an exponentially increasing concentration of the peptide comprising the antigen. In some embodiments, the CD137-expressing enriched cells are subjected to an increasing concentration of the peptide comprising the antigen, wherein the concentration of the peptide is 2-200 fold higher each subsequent times over a previous concentration. In some embodiments, the peptide concentration may be 1.1-100 fold higher each subsequent times over a previous concentration. In some embodiments, the peptide concentration may be 1.1-90 fold higher each subsequent times over a previous concentration. In some embodiments, the peptide concentration may be 1.1-80 fold higher each subsequent times over a previous concentration. In some embodiments, the peptide concentration may be 1.1-70 fold higher each subsequent times over a previous concentration. In some embodiments, the peptide concentration may be 1.1-60 fold higher each subsequent times over a previous concentration. In some embodiments, the peptide concentration may be 1.1-50 fold higher each subsequent times over a previous concentration. In some embodiments, the peptide concentration may be 1.1-40 fold higher each subsequent times over a previous concentration. In some embodiments, the peptide concentration may be 2-30 fold higher each subsequent times over a previous concentration. In some embodiments, the peptide concentration may be 2-20 fold higher each subsequent times over a previous concentration. In some embodiments, the peptide concentration may be 2-10 fold higher each subsequent times over a previous concentration. In some embodiments, the increasing concentration of the peptide antigen in the culture medium comprises a concentration that increases by at least 1.1, 2, 3, 4, 5, 6, 7, 8, 9 or 10 fold over the beginning concentration that is at least 2 fold lower than the concentration of the peptide antigen in the first or the second culture medium

In some embodiments, the concentration of the peptide in the first stimulation with the peptide of the one, two, three or more periods of time in the pulse phase is lower than the concentration of the peptide for the antigen spike. In some embodiments, the concentration of the peptide in the first stimulation with the peptide in the antigen pulse phase is at least 1000-fold lower than that of the antigen spike phase. In some embodiments, the concentration of the peptide in the first stimulation in the antigen pulse phase is at least 500-fold lower than the concentration of the peptide in the antigen spike phase. In some embodiments, the concentration of the peptide in the first stimulation in the antigen pulse phase is at least 200-fold lower than the concentration of the peptide in the antigen spike phase In some embodiments, the concentration of the peptide in the first stimulation in the antigen pulse phase is at least 100-fold lower than the concentration of the peptide in the antigen spike phase. In some embodiments, the concentration of the peptide in the first stimulation in the antigen pulse phase is at least 20-fold lower than the concentration of the peptide in the antigen spike phase. In some embodiments, the concentration of the peptide in the first stimulation in the antigen pulse phase is at least 10-fold lower than the concentration of the peptide in the antigen spike phase.

In some embodiments the exponential peptide antigen pulse results in an increase in antigen specific T cell expansion. In some embodiments, the antigen-specific T cell expansion is about 2 fold, 3 fold, 4 fold, 5 fold, 6 fold, 7 fold, 8 fold, 9 fold, 10 fold, about 20 fold, about 50 fold or about 100, compared to cells that have not received an exponential peptide antigen pulse.

### T Cells Enrichment and Expansion In Vitro

One aspect of the present disclosure provides methods for enrichment of antigen specific T cells and expansion of the same in vitro. In some embodiments, one method of the disclosure comprises (a) subjecting cells from a biological sample (such as a leukapheresis bag) to CD14 and CD25 cell depletion, followed by culturing the CD14 and CD25 depleted cell population. In some embodiments, the CD14− CD25− cell population is expanded initially for about 4 days, about 5 days, about 6 days, about 7 days, about 8 days, about 9 days, about 10 days, about 11 days, about 12 days, about 13 days, about 14 days or about 15 days. In some embodiments, the cells are stimulated with APCs during the expansion for about 1 day, about 2 days, about 3 days, about 4 days, about 5 days, about 6 days, or about 7 days. In some embodiments, the CD14 and CD25− depleted cell population is cultured overnight or for 2-14, 2-18 or 2-24 hours prior to stimulating with antigen. In some embodiments, the CD14 and CD25− depleted PBMC cell population is subjected to culturing in presence of antigen presenting cells that can present a peptide antigen to the T cells in the cell population. In some embodiments, culturing PBMCs in the presence of an antigen results in antigen presenting monocytes or macrophages in the PBMC population to serve as antigen presenting cells that take up the peptide antigen and present to the T cells in the population. In some embodiments, CD14− and CD25− cell population are cultured in presence of monocytes or macrophages electroporated with an mRNA encoding one or more antigenic peptides, which are presented on the monocyte or macrophage surface for T cell activation in the cultured cell population. The monocytes or macrophages or any other antigen presenting cells express an HLA that is capable or presenting the antigen to the T cells in the cell culture. In some embodiments, the CD14−, CD25− cell population that has been stimulated with antigen is subjected to an enrichment for antigen-specific T cells using flow cytometry based sorting of cells and selecting the specific cells that express one or more specific cell surface markers that are likely to enrich activated T cells. In some embodiments the one or more cell surface markers are coexpressed in activated antigen-responsive T cells. In some embodiments the antigen responsive T cells are CD8+ T cells. In some embodiments the antigen presenting cells are loaded with multiple antigens or electroporated with a polynucleotide encoding multiple antigens. In some embodiments the T cells in the population are stimulated with multiple antigens at the same time in the same culture to generate a population of heterospecific T cells, e.g., the population of cells comprise T cells that have antigen specificity to multiple antigens.

In some embodiments, the selection marker is CD137 protein. In some embodiments, CD137+ T cells are enriched via selection using anti-CD137 antibody mediated sorting and expanding the enriched cells in culture to obtain enriched and expanded antigen-specific T cells. In some embodiments, the selection marker is CD69. In some embodiments, T cells are enriched for antigen-specific T cells by selection using anti-CD69 antibody mediated cell sorting, followed by expansion of the enriched cells in vitro. In some embodiments, In some embodiments, CD137+/CD69+ T cells are enriched via selection using anti-CD137 antibody- and anti-CD69 antibody-mediated sorting and the enriched cells are expanded in culture to obtain enriched antigen-specific T cells. The methods described herein related to the enrichment process arose in part from a surprising finding that the CD137+/CD69+ enrichment and expansion of T cell is capable of enriching de novo T cells responses. Thus, in some embodiments, the method preferentially or specifically expands antigen-specific T cells. In some embodiments, the method preferentially or specifically expands naive T cells. In some embodiments, the method preferentially or specifically expands naïve antigen-specific T cells.

In some embodiments, the method comprises selection and enrichment of cells expressing CD137. The method comprises, culturing T cells from biological sample from a subject in a first cell culture medium comprising antigen presenting cells (APCs) to produce a first population of T cells, wherein the APCs present an epitope of a peptide antigen in complex with an MHC protein; culturing the first population of T cells in a second cell culture medium to produce a second population of T cells; enriching CD137 (4-1BB)-expressing T cells from the first or second population of T cells to produce a third population of T cells; and then expanding the third population of T cells in a third cell culture medium to obtain a therapeutic population of antigen-specific T cells.

In one embodiment, the cells are stimulated with a peptide antigen prior to enrichment. In one embodiment, the method comprise stimulating the cell culture at day 13 with one or more peptide antigens (antigen spike). In some embodiments, the method comprises spiking with multiple antigens. In some embodiments, the antigen spike is added in the cell culture at 1, 2, 3, 4, 5, 6, 7, 8, 9, or 10 microMolar (uM) final concentration. In some embodiments, the peptide final concentration is 10 uM or less, 8 uM or less, 5 uM or less or about 2 uM. In some embodiments, wherein cells are stimulated with multiple peptides the concentration of each peptide in the peptide spike step is about 2 uM. In some embodiments, the concentration of each peptide is about 1 uM. In some embodiments, the concentration of each peptide is 0.1 uM. In some embodiments, the antigen spike induces cell surface markers. Adequate expression of a cell surface marker that specifically expresses in activated antigen specific T cells can be ideal for enrichment using antibody that binds to the cell surface marker. In some embodiments, antigen activated T cells are subjected to enrichment at 36 hours or less from the spike peptide dose. In some embodiments, antigen activated T cells are subjected to enrichment at 24 hours or less from the spike peptide dose. In some embodiments, antigen activated T cells are subjected to enrichment at 22 hours or less from the spike peptide dose. In some embodiments, antigen activated T cells are subjected to enrichment at 20 hours or less from the spike peptide dose. In some embodiments, antigen activated T cells are subjected to enrichment at 18 hours or less from the spike peptide dose.

In some embodiments, the CD137 enrichment is performed between 12 hours and 24 hours, following spike antigen dose. In some embodiments, the CD137 enrichment is performed at about 18 hours following spike antigen dose.

In some embodiments, low antibody concentration is used for enriching CD137+ or CD69+ cells. In some embodiments, the antibody concentration is titrated to 1/5^{th}, 1/10^{th} or 1/20^{th} or 1/25^{th} of the antibody concentration generally used for this purpose.

The total cell number reduce considerably with enrichment, but the proportion of antigen-specific T cells increase in the enriched population. In some embodiments, the enrichment is done in a buffer, such as AIM-V buffer.

In some embodiments the enriched and expanded cell population comprises CD8+ T cells. In some embodiments the enriched and expanded cell population are CD8+ T cells. In some embodiments the enriched and expanded cell population comprises at least 0.1%, 0.5%, 1%, 2%, 3%, 4% or 5% CD8+ T cells. For example, the enriched and expanded cell population can comprise at least 0.1%, 0.5%, 1%, 2%, 3%, 4%, 5% 10%, 15%, 20%, 25%, 30%, 35%, 40%, 45%, 50%, 55%, 60%, 65%, 70%, 75%, 80%, 85% or 90% or more CD8+ T cells.

In some embodiments the enriched and expanded cell population comprises CD4+ T cells. In some embodiments the enriched and expanded cell population are CD4+ T cells. In some embodiments the enriched and expanded cell population comprises at least 0.1%, 0.5%, 1%, 2%, 3%, 4% or 5% CD4+ T cells. For example, the enriched and expanded cell population can comprise at least 0.1%, 0.5%, 1%, 2%, 3%, 4%, 5% 10%, 15%, 20%, 25%, 30%, 35%, 40%, 45%, 50%, 55%, 60%, 65%, 70%, 75%, 80%, 85% or 90% or more CD4+ T cells.

In some embodiments the enriched and expanded cell population comprises at least 5% CD3+ T cells. For example, the enriched and expanded cell population can comprise at least 10%, 15%, 20%, 25%, 30%, 35%, 40%, 45%, 50%, 55%, 60%, 65%, 70%, 75%, 80%, 85% or 90% or more CD3+ T cells. In some embodiments the enriched and expanded cell population comprises from 5% to 100% CD3+ T cells. For example, the enriched and expanded cell population can comprise from 10% to 100%, 15% to 100%, 20% to 100%, 25% to 100%, 30% to 100%, 35% to 100%, 40% to 45%, 50% to 100%, 55% to 100%, 65% to 100%, 70% to 100%, 75% to 100%, 80% to 100%, 85% to 100%, 90% to 100%, or 95% to 100% CD3+ T cells. For example, the enriched and expanded cell population can comprise from 10% to 90%, 15% to 90%, 20% to 90%, 25% to 90%, 30% to 90%, 35% to 90%, 40% to 45%, 50% to 90%, 55% to 90%, 65% to 90%, 70% to 90%, 75% to 90%, 80% to 90% or 85% to 90% CD3+ T cells. For example, the enriched and expanded cell population can comprise from 10% to 80%, 15% to 80%, 20% to 80%, 25% to 80%, 30% to 80%, 35% to 80%, 40% to 45%, 50% to 80%, 55% to 80%, 65% to 80%, 70% to 80% or 75% to 80% CD3+ T cells. For example, the enriched and expanded cell population can comprise from 10% to 70%, 15% to 70%, 20% to 70%, 25% to 70%, 30% to 70%, 35% to 70%, 40% to 45%, 50% to 70%, 55% to 70% or 65% to 70% CD3+ T cells.

In some embodiments the enriched and expanded cell population comprises at least 0.1%, 0.5%, 1%, 2%, 3%, 4% or 5% antigen-specific T cells. For example, the enriched and expanded cell population can comprise at least 0.1%, 0.5%, 1%, 2%, 3%, 4%, 5% 10%, 15%, 20%, 25%, 30%, 35%, 40%, 45%, 50%, 55%, 60%, 65%, 70%, 75%, 80%, 85% or 90% or more antigen-specific T cells.

In some embodiments, the enriched cells are expanded in suitable media for about 7 days, about 8 days, about 9 days, about 10 days, about 11 days, about 12 days, about 13 days, about 14 days or about 15 days.

In some embodiments, expansion of enriched T cells is done in basal media comprising about 5% human serum (HS) and in the presence of IL-2. In some embodiments, expansion of enriched T cells is done in the presence of IL-7. In some embodiments, expansion of enriched T cells is done in the presence of IL-15. In some embodiments, the expansion of enriched T cells is done in a basal media, comprising about 5% HS, one or more cytokines selected from IL-2, IL-7, and IL-15 or in the presence of one or more activators, such as CD3 or a costimulator molecule. In some embodiments, the one or more activators comprise CD28. In some embodiments one or more activators comprise CD3 and CD28. In some embodiments the CD3 is soluble CD3. In some embodiments, the CD28 is soluble CD28. In some embodiments the cell culture is stimulated by adding beads coded with CD3, or CD28 or with CD3 and CD28.

In one aspect, T cells enriched for antigen specific cells are expanded in culture followed by one or more peptide pulses. In some embodiments, the peptide may be the same peptide that was used to load APCs for stimulation of the T cells. In some embodiments a pool of peptides may be used to pulse the antigen-specific cells. In a surprising observation, it was found that a peptide pulse with an exponentially increasing dose during the expansion phase greatly enhances the yield of antigen-specific T cells. The additional stimulation during expansion phase using peptides alone is termed exponential peptide pulse in this disclosure. In some embodiments, the peptide pulse comprises an increasing dose of peptide. In some embodiments, the method for expanding T cells from a subject into a therapeutic population of antigen-specific T cells comprises: (a) culturing a T cells from biological sample from a subject in a first cell culture medium comprising antigen presenting cells (APCs), wherein the APCs present an epitope of a peptide antigen in complex with an MHC protein; (b) culturing the first population of T cells in a second cell culture medium to produce a second population of T cells; (c) optionally, enriching CD137 (4-1BB)-expressing T cells from the second population of T cells to produce a third population of T cells; and (d) expanding the second or third population of T cells in a third cell culture medium to obtain a therapeutic population of antigen-specific T cells; wherein the second and the third cell culture medium are each supplemented with a concentration of the peptide antigen, wherein the concentration of the peptide antigen in the third culture medium is at least 2 fold lower the concentration of the peptide antigen in the first culture medium and/or second culture medium. In some embodiments, the first population of T cells cultured in a second cell culture medium comprises exposing the first population of T cells to a peptide antigen spike (peptide spike) in the second culture medium, followed by enrichment. In some embodiments, the third cell culture medium is pulsed with low to high concentrations of the peptide antigen, where the first of the peptide pulses comprises at least 2-fold lower concentration than the spike concentration. In some embodiments, the peptide pulse comprises an exponentially increasing dose of peptide. In some embodiments, the peptide pulse comprises an increasing dose of peptide, ranging from 0.01 μM to 10 μM peptides. In some embodiments, the peptide pulse comprises an increasing dose of peptide, ranging from 0.05 μM to 10 μM peptides. In some embodiments, the peptide pulse comprises an increasing dose of peptide, ranging from 0.1 μM to 10 μM peptides. In some embodiments the peptide pulse comprises about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM or 10 μM peptide. In some embodiments the peptide pulse comprises exponentially increasing dose, from about 0.1 μM, then 0.4 μM and then 1 μM peptide. In some embodiments an exponential peptide pulse is administered to the cells in culture for 2, 3, 4, 5, or 6 times. In some embodiments, exponential peptide pulse is administered to the cells in culture for more than 6 times. In some embodiments, exponential peptide pulse is administered to the cells for 2 times. In some embodiments, exponential peptide pulse is administered to the cells for 3 times.

In some embodiments, culturing a first population of T cells in a first or second cell culture medium comprises culturing the first population of T cells with 0.01 μM to 10 μM of peptide. In some embodiments, culturing a first population of T cells in a first or second cell culture medium comprises culturing the first population of T cells with 0.05 μM to 10 μM peptide. In some embodiments, culturing a first population of T cells in a first or second cell culture medium comprises culturing the first population of T cells with 0.1 μM to 10 μM peptide. In some embodiments, culturing a first population of T cells in a first or second cell culture medium comprises culturing the first population of T cells with about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM or 10 μM peptide. In some embodiments the peptide is a peptide consisting of the epitope. In some embodiments, the peptide is a peptide comprising the epitope and one or more additional amino acids, such 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more additional amino acids. In some embodiments, culturing the first population of T cells in a first or second cell culture medium comprises culturing the first population of T cells in the presence of one or more peptides, comprising 2, 3, 4, 5, 6, 7, 8, 9, 10 or more different antigens.

In some embodiments, expanding the second population of T cells or the enriched second population of T cells in a second or third cell culture medium comprises culturing the second population of T cells or the enriched second population of T cells with 0.01 μM to 10 μM, 0.05 μM to 10 μM, or 0.1 μM to 10 μM of peptide. In some embodiments, expanding the second population of T cells or the enriched second population of T cells in a second or third cell culture medium comprises culturing the second population of T cells or the enriched second population of T cells with 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM or 10 μM peptide.

In some embodiments, supplementing the second cell culture medium comprises supplementing the second or third cell culture medium with 0.01 μM to 10 μM, 0.05 μM to 10 μM, or 0.1 μM to 10 μM of peptide. In some embodiments, supplementing the second cell culture medium comprises supplementing the second cell culture medium with 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM or 10 μM peptide. In some embodiments, supplementing the second cell culture medium comprises supplementing the second cell culture medium with a first amount of peptide and further supplementing the second cell culture medium with a second amount of peptide that is higher than the first amount of peptide. In some embodiments, the method further comprises supplementing the second cell culture medium with a third amount of peptide that is higher than the second amount of peptide. In some embodiments, the method further comprises supplementing the second cell culture medium with a fourth amount of peptide that is higher than the third amount of peptide. In some embodiments, the method further comprises supplementing the second cell culture medium with a fifth amount of peptide that is higher than the fourth amount of peptide.

In some embodiments, expanding the enriched second population of T cells in a second cell culture medium comprises expanding the enriched second population of T cells in a second cell culture medium with 0.01 μM to 10 μM, 0.05 μM to 10 μM, or 0.1 μM to 10 μM of peptide. In some embodiments, expanding the enriched second population of T cells in a second cell culture medium comprises expanding the enriched second population of T cells in a second cell culture medium with 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM or 10 μM peptide. In some embodiments, expanding the enriched second population of T cells in a second cell culture medium comprises expanding the enriched second population of T cells in a second cell culture medium with a first amount of peptide and supplementing the second cell culture medium with a second amount of peptide that is higher than the first amount of peptide. In some embodiments, the method further comprises supplementing the second cell culture medium with a third amount of peptide that is higher than the second amount of peptide. In some embodiments, the method further comprises supplementing the second cell culture medium with a fourth amount of peptide that is higher than the third amount of peptide. In some embodiments, the method further comprises supplementing the second cell culture medium with a fifth amount of peptide that is higher than the fourth amount of peptide.

In some embodiments, the antigen spike is added on day 13 and cells are enriched on day 14. In some embodiments, the peptide pulse is added on day 15, 16 and 17. In some embodiments, the cells are not enriched.

In some embodiments, one or more cytokines and/or growth factors are added in the cell culture at any point between day 0 and day 26. In some embodiments, the growth factor includes serum. In some embodiments, the serum is human serum. In some embodiments, the cytokines include IL7, or IL15 or both.

In some embodiments, the expanded T cells are harvested before 30 days starting from obtaining the cells from the biological sample (Day 0). In some embodiments, the expanded T cells are harvested at 29 days or less, at 28 days or less, at 27 days or less, at 26 days or less, at 25 days or less, at 24 days or less, or at 23 days or less. In some embodiments, the enriched and expanded T cells are collected at 26 days or less from Day 0.

In some embodiments, the enrichment, expansion and/or harvesting is done under aseptic conditions, in a closed system.

### Methods of Treating

Provided herein is a method for treating cancer in a subject, comprising: I. contacting cancer neoantigen loaded antigen presenting cells (APCs) with isolated T cells ex vivo, wherein, the cancer neoantigen loaded antigen presenting cells (APCs) are CD11b depleted; II. preparing cancer neoantigen primed T cells for a cellular composition for cancer immunotherapy ex vivo; and III. administering the cellular composition for cancer immunotherapy in the subject, wherein at least one or more conditions or symptoms related to the cancer are reduced or ameliorated by the administering, thereby treating the subject, wherein the cancer neoantigen loaded APCs and the cancer neoantigen primed T cells each express a protein encoded by an HLA allele that is expressed in the subject, and to which the neoantigen can specifically bind.

In some embodiments, the method further comprises administering one or more of the at least one antigen specific T cell to a subject. In some embodiments, the therapeutic composition comprising T cells is administered by injection. In some embodiments, the therapeutic composition comprising T cells is administered by infusion. When administration is by injection, the active agent can be formulated in aqueous solutions, specifically in physiologically compatible buffers such as Hanks solution, Ringer's solution, or physiological saline buffer. The solution can contain formulator agents such as suspending, stabilizing and/or dispersing agents. In another embodiment, the pharmaceutical composition does not comprise an adjuvant or any other substance added to enhance the immune response stimulated by the peptide. In some embodiments, the method further comprises administering one or more of the at least one antigen specific T cell as a pharmaceutical composition described herein to a subject. In some embodiments, the pharmaceutical composition comprises a preservative or stabilizer. In some embodiments the preservative or stabilizer is selected from a cytokine, a growth factor or an adjuvant or a chemical substance. In some embodiments, the at least one antigen specific T cell is administered to a subject within 28 days from collecting a PBMC sample from the subject.

In addition to the formulations described previously, the active agents can also be formulated as a depot preparation. Such long acting formulations can be administered by implantation or transcutaneous delivery (for example subcutaneously or intramuscularly), intramuscular injection or use of a transdermal patch. Thus, for example, the agents can be formulated with suitable polymeric or hydrophobic materials (for example as an emulsion in an acceptable oil) or ion exchange resins, or as sparingly soluble derivatives, for example, as a sparingly soluble salt.

Also provided herein are methods of treating a subject with a disease, disorder or condition. A method of treatment can comprise administering a composition or pharmaceutical composition disclosed herein to a subject with a disease, disorder or condition.

The present disclosure provides methods of treatment comprising an immunogenic therapy. Methods of treatment for a disease (such as cancer or a viral infection) are provided. A method can comprise administering to a subject an effective amount of a composition comprising an immunogenic antigen specific T cells according to the methods provided herein. In some embodiments, the antigen comprises a viral antigen. In some embodiments, the antigen comprises a tumor antigen.

Non-limiting examples of therapeutics that can be prepared include a peptide-based therapy, a nucleic acid-based therapy, an antibody based therapy, a T cell based therapy, and an antigen-presenting cell based therapy.

In some other aspects, provided here is use of a composition or pharmaceutical composition for the manufacture of a medicament for use in therapy. In some embodiments, a method of treatment comprises administering to a subject an effective amount of T cells specifically recognizing an immunogenic neoantigen peptide. In some embodiments, a method of treatment comprises administering to a subject an effective amount of a TCR that specifically recognizes an immunogenic neoantigen peptide, such as a TCR expressed in a T cell.

In some embodiments, the cancer is selected from the group consisting of carcinoma, lymphoma, blastoma, sarcoma, leukemia, squamous cell cancer, lung cancer (including small cell lung cancer, non-small cell lung cancer (NSCLC), adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblasts leukemia, and post-transplant lymphoproliferative disorder (PTLD), abnormal vascular proliferation associated with phakomatoses, edema, Meigs' syndrome, and combinations thereof.

The methods described herein are particularly useful in the personalized medicine context, where immunogenic neoantigen peptides identified according to the methods described herein are used to develop therapeutics (such as vaccines or therapeutic antibodies) for the same individual. Thus, a method of treating a disease in a subject can comprise identifying an immunogenic neoantigen peptide in a subject according to the methods described herein; and synthesizing the peptide (or a precursor thereof, such as a polynucleotide (e.g., an mRNA) encoding the peptide); and manufacturing T cells specific for identified neoantigens; and administering the neoantigen specific T cells to the subject. In some embodiments, the method of treating a disease in a subject can comprise identifying an immunogenic neoantigen peptide in a subject according to the methods described herein; and synthesizing the polynucleotide, such as an mRNA, that encodes the immunogenic neoantigen peptide or a precursor thereof, and manufacturing T cells specific for identified neoantigens; and administering the neoantigen specific T cells to the subject.

The agents and compositions provided herein may be used alone or in combination with conventional therapeutic regimens such as surgery, irradiation, chemotherapy and/or bone marrow transplantation (autologous, syngeneic, allogeneic or unrelated). A set of tumor antigens can be identified using the methods described herein and are useful, e.g., in a large fraction of cancer patients.

In some embodiments, at least one or more chemotherapeutic agents may be administered in addition to the composition comprising an immunogenic therapy. In some embodiments, the one or more chemotherapeutic agents may belong to different classes of chemotherapeutic agents.

In practicing the methods of treatment or use provided herein, therapeutically-effective amounts of the therapeutic agents can be administered to a subject having a disease or condition. A therapeutically-effective amount can vary widely depending on the severity of the disease, the age and relative health of the subject, the potency of the compounds used, and other factors.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (i.e. a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

In some embodiments, the disease or condition that can be treated with the methods disclosed herein is cancer. Cancer is an abnormal growth of cells which tend to proliferate in an uncontrolled way and, in some cases, to metastasize (spread). A tumor can be cancerous or benign. A benign tumor means the tumor can grow but does not spread. A cancerous tumor is malignant, meaning it can grow and spread to other parts of the body. If a cancer spreads (metastasizes), the new tumor bears the same name as the original (primary) tumor.

The methods of the disclosure can be used to treat any type of cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but not limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In some embodiments, the treatment with adoptive T cells generated by the method described herein is directed to treatment of a specific patient population. In some embodiments, the adoptive T cells are directed to treatment of population of patients that are refractory to a certain therapy. For example, the T cells are directed to treatment of population of patients that are refractory to anti-checkpoint inhibitor therapy. In some embodiments, the patient is a melanoma patient. In some embodiments, the patient is a metastatic melanoma patient. In some embodiments, provided herein are methods of treating unresectable melanoma patient. In some embodiments, unresectable melanoma patients are selected for the T cell therapy described herein (such as NEO-PTC-01). Unresectable melanoma subjects may not be candidates for therapy with tumor infiltrating lymphocytes. In some embodiments, the treatment with adoptive T cells generated by the method described herein is directed to treatment of metatstatic and unresectable melanoma patients. In some embodiments, the patient is refractory to anti-PD1 therapy. In some embodiments, the patient is refractory to anti-CTLA-4 therapy. In some embodiments, the patient is refractory to both anti-PD1 and anti-CTLA-4 therapy. In some embodiments, the therapy is administered by intravenously. In some embodiments, the therapy is administered by injection or infusion. In some embodiments the therapy is administered via a single dose, or 2, 3, 4, 5, 6, 7, 8, 9 or 10 doses. In some embodiments, the therapeutic or pharmaceutical composition comprises about 10\hat{ }9 or higher total number of cells per dose. In some embodiments, the therapeutic or pharmaceutical composition comprises 10\hat{ }10 or higher total number of cells per dose. In some embodiments, the therapeutic or pharmaceutical composition comprises 10\hat{ }11 or higher total number of cells per dose. In some embodiments, the therapeutic or pharmaceutical composition comprises 10\hat{ }12 or higher total number of cells per dose. In some embodiments, the subject is administered a therapeutic composition as described herein having about 10\hat{ }10 to about 10\hat{ }11 total cells per dose, wherein the cells have been validated for quality and have passed the release criteria.

### Kits

The methods and compositions described herein can be provided in kit form together with instructions for administration. Typically, the kit can include the desired neoantigen therapeutic compositions in a container, in unit dosage form and instructions for administration. Additional therapeutics, for example, cytokines, lymphokines, checkpoint inhibitors, antibodies, can also be included in the kit. Other kit components that can also be desirable include, for example, a sterile syringe, booster dosages, and other desired excipients.

Kits and articles of manufacture are also provided herein for use with one or more methods described herein. The kits can contain one or more types of immune cells. The kits can also contain reagents, peptides, and/or cells that are useful for antigen specific immune cell (e.g. neoantigen specific T cells) production as described herein. The kits can further contain adjuvants, reagents, and buffers necessary for the makeup and delivery of the antigen specific immune cells.

The kits can also include a carrier, package, or container that is compartmentalized to receive one or more containers such as vials, tubes, and the like, each of the container(s) comprising one of the separate elements, such as the polypeptides and adjuvants, to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. A kit typically includes labels listing contents and/or instructions for use, and package inserts with instructions for use. A set of instructions can also be included.

### EXAMPLES

The present disclosure will be described in greater detail by way of the following specific examples. The following examples are offered for illustrative purposes, and are not intended to limit the invention in any manner Those of skill in the art will readily recognize a variety of non-critical parameters that can be changed or modified to yield alternative embodiments according to the invention. All patents, patent applications, and printed publications listed herein are incorporated herein by reference in their entirety.

### Example 1. T Cell Manufacturing for Therapeutic Applications

In this example, a T cell manufacturing process (GMP) is described. An exemplary manufacturing process is summarized in FIG. 1. This process can be followed with minor revisions by a manufacturing organization under contract, to manufacture phase 1/II clinical product for NEO-STC-01 program. NEO-STC-01 is an autologous neoantigen-specific adoptive T cell therapy, comprising primarily CD3+ T cells that have been expanded ex vivo with autologous antigen-presenting cells pulsed with specific antigens, for example, KRAS-specific neoantigen peptides.

The process steps are listed and described below:

One bag of autologous leukapheresis is provided for each individualized patient manufacturing run.

There will be four separate mutation-specific KRAS neoantigen peptide pools. Each patient manufacturing run will require one of these pools based upon the patient's mutation profile.

Between one and six separate cultures are carried out in parallel during the induction phase of the manufacturing process.

Upon completion of induction, all cultures are pooled and a cell selection procedure is performed to enrich the pool for the product target cells.

The enriched product pool is then expanded in one or two cultures and later harvested and formulated.

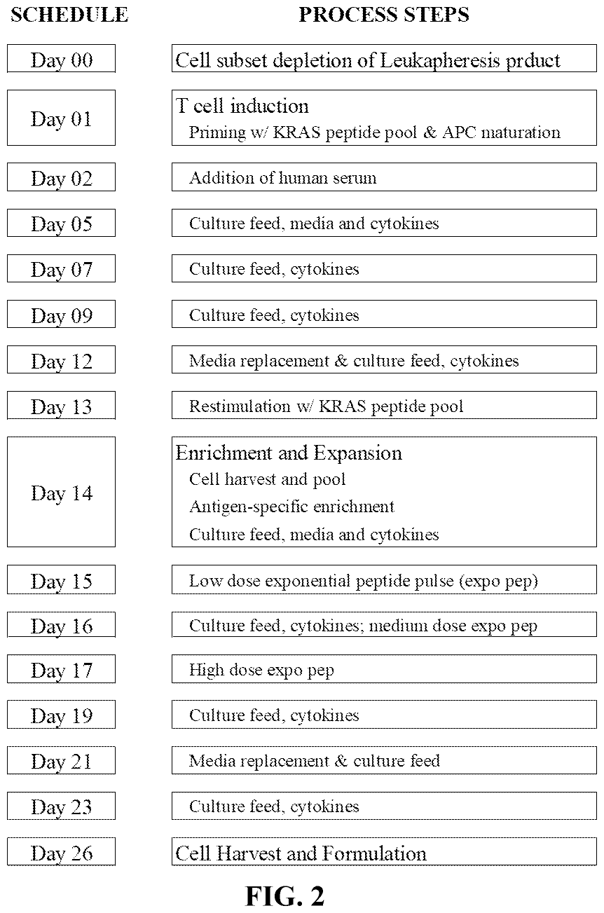

In process controls are carried out for several steps of the process and include cell count, cell viability, and cell phenotyping. An exemplary schedule is shown in FIG. 1A, FIG. 1B, and FIG. 2.

Materials supplied at the initiation of the process: Autologous peripheral blood mononuclear cells (PBMC) from the subject, cryopreserved; KRAS-specific neoantigen peptide pools in DMSO, frozen at −80° C.

Media and Reagents procured: AIM-V® medium; Human Serum Albumin; CliniMACS cell depletion reagents, Miltenyi Biotec; CliniMACS PBS/EDTA buffer; Pulmozyme®; Flt3 ligand; Tumor Necrosis Factor alpha (TNFα); Interleukin 1 Beta (IL-1b); Interleukin 7 (IL-7); Interleukin 15 (IL-15); Prostaglandin E1 (PGE-1); Human Serum (allogeneic, male AB, pooled); Normal saline for injection; CryoStor® CS10.

Equipment and Consumables: CliniMACS® Plus, Miltenyi Biotec; Cell counter; Flow cytometer capable of monitoring four concurrent wavelengths; Centrifuge; Plasma press; Cell culture incubator; 0.2 μm, DMSO compatible, syringe filters; G-Rex® 10M-CS and 100M-CS Gas Permeable Cell Culture Devices; GatheRex™ Liquid Handling, Cell Harvest Pump; Control rate freezer; CryoMACS® Freezing Bags.

Product Specifications:

The final product is a single infusion bag containing approximately 200 mL of product at a target concentration of 1×10⁹ cells/mL for a total cell count of 10×10⁹ cells. The product is frozen and stored at <−140° C. Exemplary product specification are as in Table A.

TABLE A

| Test | Method | Acceptance Criteria |
|---|---|---|
| Appearance | Visual examination | Bag integrity confirmed. |
| | | Cell suspension, visibly |
| | | free from foreign matter. |
| Cell count | Cell counter | Concentration >30 cells/mL |
| | | Target of 10 × 10⁹ cells, |
| | | acceptance range to be |
| | | determined (tbd) |
| Viability | Trypan blue exclusion | ≥70% |
| %CD3+ | Flow cytometry | ≥40% |
| Residual | ELISA or MSD, tbd | tbd |
| antibody | | |
| reagent | | |
| Potency | ELISA or MSD, tbd | tbd |
| Endotoxin | Endosafe ® PTS ™ | ≤1.25 EU/mL |
| Mycoplasma | qPCR | negative |
| Sterility | BACT/ALERT ® | negative |

Safety tests are conducted with rapid methods to minimize time to product release. The current manufacturing process is estimated at 26 calendar days, with an overall turnaround time of 5 to 6 weeks at the initial phase. Due to the nature of autologous therapies and the indication (oncology), manufacturing turnaround is critical.

### Example 2. Exemplary Peptide Antigen:MHCs

Provided below are exemplary RAS peptide:MHCs that can be used in the methods described herein.

In some embodiments, a peptide comprises a RAS mutation according to Table 1 below.

TABLE 1A

| | Exemplary | | | |
|---|---|---|---|---|
| | Protein | Mutation Sequence Context | SEQ | |
| Gene | Change | (Mutated non-native residue underlined) | ID NO: | Exemplary Diseases |
| KRAS | G12C | MTEYKLVVVGACGVGKSALTIQLIQNHFVDEYDPTIEDS | 6 | BRCA (breast cancer), |
| | | YRKQVVIDGETCLLDILDTAGQE | | CESC (cervical squamous |
| | | | | cell carcinoma and |
| | | | | endocervical |
| | | | | adenocarcinoma), |
| | | | | CRC (colorectal |
| | | | | cancer), HNSC |
| | | | | (head-neck squamous |
| | | | | cell carcinoma), |
| | | | | LUAD (lung |
| | | | | adenocarcinoma), |
| | | | | PAAD (pancreatic |
| | | | | adenocarcinoma), |
| | | | | UCEC (uterine |
| | | | | corpus endometrial |
| | | | | carcinoma) |
| KRAS | G12D | MTEYKLVVVGADGVGKSALTIQLIQNHFVDEYDPTIEDS | 7 | BLCA (urothelial |
| | | YRKQVVIDGETCLLDILDTAGQE | | bladder carcinoma), |
| | | | | BRCA, CESC, CRC, |
| | | | | GBM (glioblastoma), |
| | | | | HNSC, KIRP (kidney |
| | | | | renal papillary cell |
| | | | | carcinoma), LIHC |
| | | | | (liver hepatocellular |
| | | | | carcinoma), LUAD, |
| | | | | PAAD, SKCM (skin |
| | | | | cutaneous melanoma), |
| | | | | UCEC |
| KRAS | G12R | MTEYKLVVVGARGVGKSALTIQLIQNHFVDEYDPTIEDS | 8 | PDAC, LUAD |
| | | YRKQVVIDGETCLLDILDTAGQE | | |
| KRAS | G12V | MTEYKLVVVGAVGVGKSALTIQLIQNHFVDEYDPTIEDS | 9 | BRCA, CESC, CRC, |
| | | YRKQVVIDGETCLLDILDTAGQE | | LUAD, PAAD, |
| | | | | THCA (thyroid |
| | | | | carcinoma, UCEC |
| KRAS | Q61H | AGGVGKSALTIQLIQNHFVDEYDPTIEDSYRKQVVIDGE | 10 | CRC, LUSC (lung |
| | | TCLLDILDTAGHEEYSAMRDQYMRTGEGFLCVFAINNTK | | squamous cell |
| | | SFEDIHHYREQIKRVKDSEDVPM | | carcinoma), PAAD, |
| | | | | SKCM, UCEC |
| KRAS | Q61L | AGGVGKSALTIQLIQNHFVDEYDPTIEDSYRKQVVIDGE | 11 | CRC, GBM, HNSC, |
| | | TCLLDILDTAGLEEYSAMRDQYMRTGEGFLCVFAINNTK | | LUAD, SKCM, |
| | | SFEDIHHYREQIKRVKDSEDVPM | | UCEC |

Table 1B below shows an exemplary pool of mutated RAS peptides used in the methods described herein.

TABLE 1B

| KRAS | Peptide | | SEQ | MS Validated HLA-I |
|---|---|---|---|---|
| Mutation | name | Sequence | ID NO: | Alleles |
| G12D | AS2 | GADGVGKSA | 12 | C08:02 |
| | AS3 | GADGVGKSAL | 13 | B07:02, C08:02, C03:04 |
| | AS4 | VVVGADGVGK | 14 | A03:01, A11:01, A68:01 |
| | AL1 | KKKTEYKLVVVGADGVGKSALTIQL | 15 | N/A |
| G12V | BS1 | AVGVGKSAL | 16 | C01:02 |
| | BS2 | GAVGVGKSA | 17 | C03:03, C03:04 |
| | BS3 | GAVGVGKSAL | 18 | C03:03, C03:04 |
| | BS4 | VVGAVGVGK | 19 | A03:01, A11:01, A30:01, |
| | | | | A68:01 |
| | BS5 | VVVGAVGVGK | 20 | A03:01, A11:01, A30:01, |
| | | | | A68:01 |
| | BL1 | KKKKTEYKLVVVGAVGVGKSALTIQL | 21 | N/A |
| G12R | CS2 | GARGVGKSAL | 22 | B07:02 |
| | CS3 | VVVGARGVGK | 23 | A03:01, A11:01, A30:01, |
| | | | | A68:01 |
| | CL1 | KKKTEYKLVVVGARGVGKSALTIQL | 24 | N/A |
| G12C | DS1 | VVVGACGVGK | 25 | A03:01, A11:01, A68:01 |
| | DL1 | KKKKTEYKLVVVGACGVGKSALTIQL | 26 | N/A |

In some embodiments, a peptide comprises a RAS Q61H mutation according to Table 2 below.

TABLE 2

| | SEQ | | Rank of |
|---|---|---|---|
| Peptide | ID NO: | Allele | Binding Potential |
| ILDTAGHEEY | 27 | HLA-A36:01 | 1 |
| ILDTAGHEEY | 27 | HLA-A01:01 | 2 |
| DTAGHEEYSAM | 28 | HLA-A26:01 | 3 |
| DTAGHEEYSAM | 28 | HLA-A25:01 | 4 |
| GHEEYSAM | 29 | HLA-B15:09 | 4 |
| DTAGHEEY | 30 | HLA-A26:01 | 5 |
| ILDTAGHEE | 31 | HLA-C08:02 | 5 |
| AGHEEYSAM | 32 | HLA-C01:02 | 6 |
| AGHEEYSAM | 32 | HLA-B46:01 | 6 |
| DTAGHEEY | 30 | HLA-A25:01 | 6 |
| DTAGHEEY | 30 | HLA-A01:01 | 6 |
| DTAGHEEY | 30 | HLA-B18:01 | 7 |
| DTAGHEEY | 30 | HLA-A36:01 | 7 |
| ILDTAGHEE | 31 | HLA-C05:01 | 7 |
| ILDTAGHEE | 31 | HLA-A02:07 | 7 |
| ILDTAGHEEY | 27 | HLA-A29:02 | 7 |
| ILDTAGHEEY | 27 | HLA-C08:02 | 7 |
| HEEYSAMRD | 33 | HLA-B49:01 | 8 |
| TAGHEEYSA | 34 | HLA-B35:03 | 8 |
| DTAGHEEYS | 35 | HLA-A68:02 | 9 |
| DTAGHEEYSAMR | 36 | HLA-A68:01 | 9 |
| GHEEYSAM | 29 | HLA-B39:01 | 9 |
| ILDTAGHEE | 31 | HLA-A01:01 | 9 |
| LDTAGHEEY | 37 | HLA-B53:01 | 9 |
| HEEYSAMRD | 33 | HLA-B41:01 | 10 |
| ILDTAGHEE | 31 | HLA-A36:01 | 10 |
| DTAGHEEY | 30 | HLA-B58:01 | 11 |
| LLDILDTAGH | 38 | HLA-A01:01 | 12 |
| TAGHEEYSAM | 39 | HLA-B35:03 | 12 |
| LDTAGHEEY | 37 | HLA-B35:01 | 13 |
| DILDTAGHE | 40 | HLA-A26:01 | 14 |
| DTAGHEEY | 30 | HLA-C12:03 | 14 |
| ILDTAGHEEY | 27 | HLA-C05:01 | 14 |
| AGHEEYSAM | 32 | HLA-A30:02 | 15 |
| DILDTAGHEEY | 41 | HLA-A25:01 | 15 |
| DTAGHEEY | 30 | HLA-C02:02 | 15 |
| ILDTAGHEE | 31 | HLA-C04:01 | 15 |
| DILDTAGH | 42 | HLA-A26:01 | 16 |
| ILDTAGHEE | 31 | HLA-A02:01 | 16 |
| LDTAGHEEY | 37 | HLA-A29:02 | 16 |
| ILDTAGHE | 43 | HLA-A01:01 | 17 |
| LDTAGHEEY | 37 | HLA-B18:01 | 17 |
| AGHEEYSAM | 32 | HLA-C14:03 | 18 |
| DILDTAGHEEY | 41 | HLA-A29:02 | 18 |
| DTAGHEEYS | 35 | HLA-A26:01 | 18 |
| ILDTAGHEEY | 27 | HLA-B15:01 | 18 |
| DTAGHEEYSA | 44 | HLA-A68:02 | 19 |
| ILDTAGHE | 43 | HLA-C05:01 | 19 |
| ILDTAGHEEY | 27 | HLA-A02:07 | 19 |
| ILDTAGHEEY | 27 | HLA-A30:02 | 19 |
| LDTAGHEEY | 37 | HLA-A36:01 | 19 |
| AGHEEYSAM | 32 | HLA-C14:02 | 20 |
| AGHEEYSAM | 32 | HLA-B15:03 | 20 |
| LLDILDTAGH | 38 | HLA-A02:07 | 20 |

In some embodiments, a peptide comprises a RAS Q61R mutation according to Table 3 below.

TABLE 3

| | SEQ | | Rank of |
|---|---|---|---|
| Peptide | ID NO: | Allele | Binding Potential |
| ILDTAGREEY | 45 | HLA-A36:01 | 1 |
| ILDTAGREEY | 45 | HLA-A01:01 | 2 |
| DTAGREEYSAM | 46 | HLA-A26:01 | 3 |
| DILDTAGR | 47 | HLA-A33:03 | 4 |
| DILDTAGR | 47 | HLA-A68:01 | 5 |
| DTAGREEY | 48 | HLA-A26:01 | 6 |
| DTAGREEYSAM | 46 | HLA-A25:01 | 6 |
| CLLDILDTAGR | 49 | HLA-A74:01 | 7 |
| DTAGREEY | 48 | HLA-A01:01 | 7 |
| REEYSAMRD | 50 | HLA-B41:01 | 7 |
| GREEYSAMR | 51 | HLA-B27:05 | 8 |
| ILDTAGREE | 52 | HLA-C08:02 | 8 |
| ILDTAGREEY | 45 | HLA-A29:02 | 8 |
| REEYSAMRD | 50 | HLA-B49:01 | 8 |
| AGREEYSAM | 53 | HLA-B46:01 | 9 |
| DTAGREEY | 48 | HLA-B18:01 | 9 |
| DTAGREEY | 48 | HLA-A25:01 | 9 |
| DTAGREEY | 48 | HLA-A36:01 | 9 |
| DILDTAGR | 47 | HLA-A74:01 | 10 |
| DILDTAGRE | 54 | HLA-A26:01 | 10 |
| ILDTAGREE | 52 | HLA-C05:01 | 10 |
| DILDTAGR | 47 | HLA-A26:01 | 11 |
| GREEYSAM | 55 | HLA-B39:01 | 11 |
| AGREEYSAM | 53 | HLA-B15:03 | 12 |
| GREEYSAM | 55 | HLA-C07:02 | 12 |
| ILDTAGREE | 52 | HLA-A01:01 | 12 |
| TAGREEYSA | 56 | HLA-B35:03 | 12 |
| ILDTAGREEY | 45 | HLA-A30:02 | 13 |
| DTAGREEYS | 57 | HLA-A68:02 | 14 |
| ILDTAGRE | 58 | HLA-A01:01 | 14 |
| CLLDILDTAGR | 49 | HLA-A31:01 | 15 |
| DTAGREEYSAMR | 59 | HLA-A68:01 | 15 |
| LLDILDTAGR | 60 | HLA-A01:01 | 15 |
| DTAGREEY | 48 | HLA-B58:01 | 16 |
| ILDTAGREEY | 45 | HLA-C08:02 | 16 |
| DILDTAGR | 47 | HLA-A31:01 | 17 |
| ILDTAGREE | 52 | HLA-C04:01 | 17 |
| ILDTAGREEY | 45 | HLA-A32:01 | 17 |
| LLDILDTAGR | 60 | HLA-A74:01 | 17 |
| TAGREEYSAM | 61 | HLA-B35:03 | 17 |
| DILDTAGREEY | 62 | HLA-A32:01 | 18 |
| ILDTAGRE | 58 | HLA-C05:01 | 18 |
| ILDTAGREE | 52 | HLA-A02:07 | 18 |
| REEYSAMRD | 50 | HLA-B40:01 | 18 |
| AGREEYSAM | 53 | HLA-B15:01 | 19 |
| AGREEYSAMR | 63 | HLA-A31:01 | 19 |
| ILDTAGRE | 58 | HLA-A36:01 | 19 |
| LDILDTAGR | 64 | HLA-A68:01 | 19 |
| LDTAGREEY | 65 | HLA-A29:02 | 19 |
| LDTAGREEY | 65 | HLA-B35:01 | 19 |
| REEYSAMRD | 50 | HLA-B45:01 | 19 |
| REEYSAMRDQY | 66 | HLA-A36:01 | 19 |
| DTAGREEY | 48 | HLA-C02:02 | 20 |

In some embodiments, a peptide comprises a RAS Q61K mutation according to Table 4 below.

TABLE 4

| | SEQ | | Rank of |
|---|---|---|---|
| Peptide | ID NO: | Allele | Binding Potential |
| ILDTAGKEEY | 67 | HLA-A36:01 | 1 |
| ILDTAGKEEY | 67 | HLA-A01:01 | 2 |
| DTAGKEEYSAM | 68 | HLA-A26:01 | 3 |
| CLLDILDTAGK | 69 | HLA-A03:01 | 4 |
| DTAGKEEY | 70 | HLA-A01:01 | 5 |
| DTAGKEEY | 70 | HLA-A26:01 | 5 |
| DTAGKEEYSAM | 68 | HLA-A25:01 | 5 |
| AGKEEYSAM | 71 | HLA-B46:01 | 6 |
| DILDTAGKE | 72 | HLA-A26:01 | 7 |
| KEEYSAMRD | 73 | HLA-B41:01 | 7 |
| DTAGKEEY | 70 | HLA-B18:01 | 8 |
| GKEEYSAM | 74 | HLA-B15:03 | 8 |
| ILDTAGKEE | 75 | HLA-C08:02 | 8 |
| ILDTAGKEEY | 67 | HLA-A29:02 | 8 |
| DTAGKEEYS | 76 | HLA-A68:02 | 9 |
| LDTAGKEEY | 77 | HLA-B53:01 | 9 |
| TAGKEEYSA | 78 | HLA-B35:03 | 9 |
| DILDTAGK | 79 | HLA-A68:01 | 10 |
| DTAGKEEY | 70 | HLA-A36:01 | 10 |
| KEEYSAMRD | 73 | HLA-B49:01 | 10 |
| LDTAGKEEY | 77 | HLA-C07:01 | 10 |
| DTAGKEEYSAMR | 80 | HLA-A68:01 | 11 |
| ILDTAGKEE | 75 | HLA-C05:01 | 11 |
| ILDTAGKEEY | 67 | HLA-C08:02 | 11 |
| LLDILDTAGK | 81 | HLA-A01:01 | 12 |
| AGKEEYSAM | 71 | HLA-A30:02 | 13 |
| DTAGKEEY | 70 | HLA-A25:01 | 13 |
| DTAGKEEYS | 76 | HLA-A26:01 | 13 |
| ILDTAGKE | 82 | HLA-C05:01 | 13 |
| LDTAGKEEY | 77 | HLA-B35:01 | 13 |
| AGKEEYSAMR | 83 | HLA-A31:01 | 14 |
| DILDTAGK | 79 | HLA-A33:03 | 14 |
| ILDTAGKE | 82 | HLA-A01:01 | 14 |
| ILDTAGKEE | 75 | HLA-A01:01 | 14 |
| ILDTAGKEE | 75 | HLA-A02:07 | 14 |
| TAGKEEYSAM | 84 | HLA-B35:03 | 14 |
| AGKEEYSAM | 71 | HLA-B15:01 | 15 |
| ILDTAGKEEY | 67 | HLA-A30:02 | 15 |
| LDTAGKEEY | 77 | HLA-B46:01 | 15 |
| DTAGKEEY | 70 | HLA-B58:01 | 16 |
| ILDTAGKEEY | 67 | HLA-C05:01 | 17 |
| AGKEEYSAM | 71 | HLA-A30:01 | 18 |
| AGKEEYSAM | 71 | HLA-B15:03 | 18 |
| DTAGKEEY | 70 | HLA-C02:02 | 18 |
| LDTAGKEEY | 77 | HLA-A29:02 | 18 |

In some embodiments, a peptide comprises a RAS Q61L mutation according to Table 5 below.

TABLE 5

| | SEQ | | Rank of |
|---|---|---|---|
| Peptide | ID NO: | Allele | Binding Potential |
| ILDTAGLEEY | 85 | HLA-A36:01 | 1 |
| ILDTAGLEEY | 85 | HLA-A01:01 | 2 |
| LLDILDTAGL | 86 | HLA-A02:07 | 3 |
| GLEEYSAMRDQY | 87 | HLA-A36:01 | 4 |
| DTAGLEEY | 88 | HLA-A25:01 | 5 |
| DTAGLEEY | 88 | HLA-A26:01 | 5 |
| DTAGLEEYSAM | 89 | HLA-A26:01 | 5 |
| DTAGLEEY | 88 | HLA-A01:01 | 6 |
| ILDTAGLEE | 90 | HLA-C08:02 | 6 |
| ILDTAGLEE | 90 | HLA-A01:01 | 6 |
| CLLDILDTAGL | 91 | HLA-A02:04 | 7 |
| ILDTAGLEE | 90 | HLA-A36:01 | 7 |
| LLDILDTAGL | 86 | HLA-A01:01 | 7 |
| DILDTAGL | 92 | HLA-B14:02 | 8 |
| DILDTAGLEEY | 93 | HLA-A25:01 | 8 |
| DTAGLEEYS | 94 | HLA-A68:02 | 8 |
| DTAGLEEYSAM | 89 | HLA-A25:01 | 8 |
| GLEEYSAMR | 95 | HLA-A74:01 | 8 |
| ILDTAGLE | 96 | HLA-A01:01 | 8 |
| DILDTAGLEEY | 93 | HLA-A26:01 | 9 |
| DTAGLEEY | 88 | HLA-A36:01 | 9 |
| ILDTAGLEEY | 85 | HLA-A29:02 | 9 |
| DILDTAGL | 92 | HLA-B08:01 | 10 |
| DTAGLEEY | 88 | HLA-B18:01 | 10 |
| ILDTAGLEE | 90 | HLA-A02:07 | 10 |
| LDTAGLEEY | 97 | HLA-B35:01 | 10 |
| CLLDILDTAGL | 91 | HLA-A02:01 | 11 |
| DTAGLEEY | 88 | HLA-C02:02 | 11 |
| ILDTAGLEE | 90 | HLA-C05:01 | 11 |
| ILDTAGLEEY | 85 | HLA-C08:02 | 11 |
| ILDTAGLEEY | 85 | HLA-A02:07 | 11 |
| LLDILDTAGL | 86 | HLA-C08:02 | 11 |
| DILDTAGL | 92 | HLA-A26:01 | 12 |
| LDTAGLEEY | 97 | HLA-B53:01 | 12 |
| DTAGLEEY | 88 | HLA-C03:02 | 13 |
| DTAGLEEY | 88 | HLA-B58:01 | 13 |
| ILDTAGLEEY | 85 | HLA-A30:02 | 13 |
| LLDILDTAGL | 86 | HLA-C05:01 | 13 |
| LLDILDTAGL | 86 | HLA-C04:01 | 13 |
| DTAGLEEYSAMR | 98 | HLA-A68:01 | 14 |
| ILDTAGLE | 96 | HLA-A36:01 | 15 |
| LLDILDTAGL | 86 | HLA-A02:01 | 15 |
| AGLEEYSAM | 99 | HLA-B15:03 | 16 |
| DTAGLEEYSA | 100 | HLA-A68:02 | 16 |
| GLEEYSAMRDQY | 87 | HLA-A01:01 | 16 |
| ILDTAGLE | 96 | HLA-C04:01 | 16 |
| ILDTAGLEEY | 85 | HLA-B15:01 | 16 |
| LDILDTAGL | 101 | HLA-B37:01 | 16 |
| AGLEEYSAM | 99 | HLA-A30:02 | 17 |
| AGLEEYSAM | 99 | HLA-B48:01 | 17 |
| AGLEEYSAMR | 102 | HLA-A31:01 | 17 |
| ILDTAGLEE | 90 | HLA-C04:01 | 17 |
| LDTAGLEEY | 97 | HLA-C03:02 | 17 |
| AGLEEYSAM | 99 | HLA-C14:02 | 18 |
| GLEEYSAMR | 95 | HLA-A31:01 | 18 |
| LEEYSAMRD | 103 | HLA-B41:01 | 18 |
| LLDILDTAGLE | 104 | HLA-A01:01 | 18 |
| AGLEEYSAM | 99 | HLA-C14:03 | 19 |
| LDILDTAGL | 101 | HLA-B40:02 | 19 |
| LDTAGLEEY | 97 | HLA-A29:02 | 19 |
| DILDTAGLE | 105 | HLA-A26:01 | 20 |
| DTAGLEEY | 88 | HLA-B15:01 | 20 |
| ILDTAGLEEY | 85 | HLA-A02:01 | 20 |
| LDTAGLEEY | 97 | HLA-A36:01 | 20 |
| LDTAGLEEY | 97 | HLA-B46:01 | 20 |
| DTAGLEEY | 88 | HLA-A68:02 | 21 |
| DTAGLEEY | 88 | HLA-C12:03 | 21 |
| ILDTAGLE | 96 | HLA-C05:01 | 21 |
| LDTAGLEEY | 97 | HLA-B18:01 | 21 |
| LEEYSAMRD | 103 | HLA-B49:01 | 21 |
| TAGLEEYSA | 106 | HLA-B54:01 | 21 |
| DILDTAGLEEY | 93 | HLA-A29:02 | 22 |
| GLEEYSAM | 107 | HLA-C05:01 | 22 |

In some embodiments, a peptide comprises a RAS G12A mutation according to Table 6 below.

TABLE 6

| | SEQ | | Rank of |
|---|---|---|---|
| Peptide | ID NO: | Allele | Binding Potential |
| AAGVGKSAL | 108 | HLA-C03:04 | 1 |
| VVVGAAGVGK | 109 | HLA-A11:01 | 1 |
| VVGAAGVGK | 110 | HLA-A11:01 | 2 |
| TEYKLVVVGAA | 111 | HLA-B50:01 | 3 |
| VVGAAGVGK | 110 | HLA-A03:01 | 3 |
| VVVGAAGVGK | 109 | HLA-A68:01 | 3 |
| AAGVGKSAL | 108 | HLA-C08:02 | 4 |
| AAGVGKSAL | 108 | HLA-C08:01 | 4 |
| AAGVGKSAL | 108 | HLA-B46:01 | 4 |
| AAGVGKSAL | 108 | HLA-B81:01 | 5 |
| GAAGVGKSAL | 112 | HLA-B48:01 | 5 |
| LVVVGAAGV | 113 | HLA-A68:02 | 5 |
| AAGVGKSAL | 108 | HLA-C03:04 | 1 |
| VVVGAAGVGK | 109 | HLA-A11:01 | 1 |
| VVGAAGVGK | 110 | HLA-A11:01 | 2 |
| TEYKLVVVGAA | 111 | HLA-B50:01 | 3 |
| VVGAAGVGK | 110 | HLA-A03:01 | 3 |
| VVVGAAGVGK | 109 | HLA-A68:01 | 3 |
| AAGVGKSAL | 108 | HLA-C08:02 | 4 |
| AAGVGKSAL | 108 | HLA-C08:01 | 4 |
| AAGVGKSAL | 108 | HLA-B46:01 | 4 |
| AAGVGKSAL | 108 | HLA-B81:01 | 5 |
| AAGVGKSAL | 108 | HLA-C03:02 | 5 |
| AAGVGKSAL | 108 | HLA-C01:02 | 5 |
| GAAGVGKSAL | 112 | HLA-B48:01 | 5 |
| LVVVGAAGV | 113 | HLA-A68:02 | 5 |
| AAGVGKSAL | 108 | HLA-C03:03 | 6 |
| VVGAAGVGK | 110 | HLA-A68:01 | 6 |
| GAAGVGKSAL | 112 | HLA-B81:01 | 7 |
| VVVGAAGVGK | 109 | HLA-A03:01 | 7 |
| AAGVGKSAL | 108 | HLA-C05:01 | 8 |
| AAGVGKSAL | 108 | HLA-C12:03 | 8 |
| GAAGVGKSA | 114 | HLA-B46:01 | 8 |
| VVGAAGVGK | 110 | HLA-A30:01 | 8 |
| GAAGVGKSA | 114 | HLA-B55:01 | 9 |
| KLVVVGAAGV | 115 | HLA-A02:01 | 9 |
| AGVGKSAL | 116 | HLA-B08:01 | 10 |
| GAAGVGKSAL | 112 | HLA-C03:04 | 10 |
| AAGVGKSAL | 108 | HLA-C17:01 | 11 |
| GAAGVGKSAL | 112 | HLA-C03:03 | 11 |
| VVVGAAGV | 117 | HLA-A68:02 | 11 |
| YKLVVVGAA | 118 | HLA-B54:01 | 11 |
| AAGVGKSAL | 108 | HLA-B48:01 | 12 |
| AGVGKSAL | 116 | HLA-C03:04 | 12 |
| AGVGKSAL | 116 | HLA-C07:01 | 12 |
| VVVGAAGVGK | 109 | HLA-A30:01 | 12 |
| AAGVGKSA | 119 | HLA-B46:01 | 13 |
| KLVVVGAAGV | 115 | HLA-A02:07 | 13 |
| YKLVVVGAA | 118 | HLA-B50:01 | 13 |
| AAGVGKSAL | 108 | HLA-B07:02 | 14 |
| GAAGVGKSAL | 112 | HLA-A68:02 | 14 |
| VVGAAGVGK | 110 | HLA-A74:01 | 14 |
| AGVGKSAL | 116 | HLA-C08:01 | 15 |
| GAAGVGKSAL | 112 | HLA-C17:01 | 15 |
| GAAGVGKSAL | 112 | HLA-C08:01 | 16 |
| GAAGVGKSAL | 112 | HLA-B35:03 | 16 |
| AAGVGKSAL | 108 | HLA-C02:02 | 17 |
| AAGVGKSAL | 108 | HLA-B35:03 | 17 |
| AAGVGKSAL | 108 | HLA-C12:02 | 17 |
| AAGVGKSAL | 108 | HLA-C14:03 | 17 |
| GAAGVGKSA | 114 | HLA-B50:01 | 17 |
| AGVGKSAL | 116 | HLA-C03:02 | 18 |
| GAAGVGKSA | 114 | HLA-C03:04 | 18 |
| LVVVGAAGV | 113 | HLA-B55:01 | 18 |
| TEYKLVVVGAA | 111 | HLA-B41:01 | 18 |
| AGVGKSAL | 116 | HLA-C01:02 | 19 |
| GAAGVGKSA | 114 | HLA-B54:01 | 19 |
| GAAGVGKSAL | 112 | HLA-B07:02 | 19 |
| VGAAGVGKSA | 120 | HLA-B55:01 | 19 |
| AGVGKSAL | 116 | HLA-B48:01 | 20 |
| AGVGKSALTI | 121 | HLA-B49:01 | 20 |
| VVVGAAGV | 117 | HLA-B55:01 | 20 |

In some embodiments, a peptide comprises a RAS G12C mutation according to Table 7 below.

TABLE 7

| | SEQ | | Rank of |
|---|---|---|---|
| Peptide | ID NO: | Allele | Binding Potential |
| VVVGACGVGK | 25 | HLA-A11:01 | 1 |
| VVGACGVGK | 122 | HLA-A03:01 | 2 |
| VVGACGVGK | 122 | HLA-A11:01 | 3 |
| VVVGACGVGK | 25 | HLA-A68:01 | 4 |
| VVGACGVGK | 122 | HLA-A68:01 | 5 |
| VVVGACGVGK | 25 | HLA-A03:01 | 5 |
| VVGACGVGK | 122 | HLA-A30:01 | 6 |
| ACGVGKSAL | 123 | HLA-B81:01 | 7 |
| ACGVGKSAL | 123 | HLA-C01:02 | 7 |
| ACGVGKSAL | 123 | HLA-C14:03 | 8 |
| ACGVGKSAL | 123 | HLA-C03:04 | 9 |
| VVVGACGVGK | 25 | HLA-A30:01 | 9 |
| ACGVGKSAL | 123 | HLA-C14:02 | 10 |
| CGVGKSAL | 124 | HLA-B08:01 | 10 |
| KLVVVGACGV | 125 | HLA-A02:01 | 10 |
| ACGVGKSAL | 123 | HLA-B07:02 | 11 |
| GACGVGKSAL | 126 | HLA-B48:01 | 12 |
| GACGVGKSAL | 126 | HLA-C03:03 | 13 |
| ACGVGKSAL | 123 | HLA-B48:01 | 14 |
| ACGVGKSAL | 123 | HLA-B40:01 | 14 |
| YKLVVVGAC | 127 | HLA-B48:01 | 14 |
| YKLVVVGAC | 127 | HLA-B15:03 | 14 |
| GACGVGKSA | 128 | HLA-B46:01 | 15 |
| GACGVGKSAL | 126 | HLA-C03:04 | 15 |
| GACGVGKSAL | 126 | HLA-C01:02 | 15 |
| LVVVGACGV | 129 | HLA-A68:02 | 15 |
| CGVGKSAL | 124 | HLA-C03:04 | 16 |
| GACGVGKSAL | 126 | HLA-C08:02 | 16 |
| VVGACGVGK | 122 | HLA-A74:01 | 16 |

In some embodiments, a peptide comprises a RAS G12D mutation according to Table 8 below.

TABLE 8

| | SEQ | | Rank of |
|---|---|---|---|
| Peptide | ID NO: | Allele | Binding Potential |
| GADGVGKSAL | 13 | HLA-C08:02 | 1 |
| GADGVGKSAL | 13 | HLA-C05:01 | 2 |
| VVVGADGVGK | 14 | HLA-A11:01 | 3 |
| DGVGKSAL | 130 | HLA-B14:02 | 4 |
| VVGADGVGK | 131 | HLA-A11:01 | 4 |
| VVGADGVGK | 131 | HLA-A03:01 | 5 |
| DGVGKSAL | 130 | HLA-B08:01 | 6 |
| VVVGADGVGK | 14 | HLA-A68:01 | 6 |
| GADGVGKSAL | 13 | HLA-C03:03 | 7 |
| VVGADGVGK | 131 | HLA-A30:01 | 7 |
| ADGVGKSAL | 132 | HLA-B37:01 | 8 |
| GADGVGKSAL | 13 | HLA-C08:01 | 8 |
| VVGADGVGK | 131 | HLA-A68:01 | 8 |
| GADGVGKSA | 12 | HLA-C08:02 | 9 |
| GADGVGKSAL | 13 | HLA-B35:03 | 9 |
| GADGVGKS | 133 | HLA-C05:01 | 10 |
| GADGVGKSA | 12 | HLA-C05:01 | 10 |
| ADGVGKSAL | 132 | HLA-C07:01 | 11 |
| VVVGADGVGK | 14 | HLA-A03:01 | 11 |
| ADGVGKSAL | 132 | HLA-B40:02 | 12 |
| ADGVGKSAL | 132 | HLA-B46:01 | 13 |
| GADGVGKSAL | 13 | HLA-C03:04 | 13 |
| ADGVGKSAL | 132 | HLA-B81:01 | 14 |
| GADGVGKSAL | 13 | HLA-C17:01 | 14 |
| VVVGADGVGK | 14 | HLA-A30:01 | 14 |
| GADGVGKSA | 12 | HLA-B35:03 | 15 |
| GADGVGKSA | 12 | HLA-B46:01 | 15 |
| GADGVGKSAL | 13 | HLA-B48:01 | 15 |
| KLVVVGADGV | 134 | HLA-A02:01 | 15 |
| LVVVGADGV | 135 | HLA-A68:02 | 15 |
| VGADGVGKSA | 136 | HLA-B55:01 | 15 |
| VVGADGVGK | 131 | HLA-A74:01 | 16 |
| GADGVGKSA | 12 | HLA-B53:01 | 17 |
| KLVVVGADGV | 134 | HLA-A02:07 | 17 |
| VGADGVGK | 137 | HLA-A68:01 | 17 |
| YKLVVVGAD | 138 | HLA-B48:01 | 17 |
| ADGVGKSAL | 132 | HLA-C14:03 | 18 |
| DGVGKSALTI | 139 | HLA-B51:01 | 18 |
| VGADGVGK | 137 | HLA-A11:01 | 18 |

In some embodiments, a peptide comprises a RAS G12R mutation according to Table 9 below.

TABLE 9

| | SEQ | | Rank of |
|---|---|---|---|
| Peptide | ID NO: | Allele | Binding Potential |
| VVGARGVGK | 140 | HLA-A03:01 | 1 |
| EYKLVVVGAR | 141 | HLA-A33:03 | 2 |
| VVVGARGVGK | 23 | HLA-A11:01 | 3 |
| ARGVGKSAL | 142 | HLA-C07:02 | 4 |
| ARGVGKSAL | 142 | HLA-B39:01 | 5 |
| ARGVGKSAL | 142 | HLA-C07:01 | 5 |
| VVGARGVGK | 140 | HLA-A11:01 | 5 |
| VVVGARGVGK | 23 | HLA-A68:01 | 5 |
| GARGVGKSA | 143 | HLA-B46:01 | 6 |
| ARGVGKSAL | 142 | HLA-B27:05 | 7 |
| GARGVGKSA | 143 | HLA-B55:01 | 7 |
| RGVGKSAL | 144 | HLA-C07:01 | 8 |
| VVGARGVGK | 140 | HLA-A30:01 | 9 |
| ARGVGKSAL | 142 | HLA-B38:01 | 10 |
| ARGVGKSAL | 142 | HLA-B14:02 | 10 |
| VVGARGVGK | 140 | HLA-A68:01 | 10 |
| VVVGARGVGK | 23 | HLA-A03:01 | 11 |
| GARGVGKSAL | 22 | HLA-B48:01 | 12 |
| RGVGKSAL | 144 | HLA-B48:01 | 12 |
| RGVGKSALTI | 145 | HLA-A23:01 | 12 |
| ARGVGKSAL | 142 | HLA-C06:02 | 13 |
| GARGVGKSA | 143 | HLA-A30:01 | 13 |
| GARGVGKSAL | 22 | HLA-B81:01 | 13 |
| VVVGARGVGK | 23 | HLA-A30:01 | 13 |
| GARGVGKSAL | 22 | HLA-B07:02 | 14 |
| LVVVGARGV | 146 | HLA-C06:02 | 14 |
| RGVGKSAL | 144 | HLA-B81:01 | 14 |
| VVGARGVGK | 140 | HLA-A74:01 | 15 |
| KLVVVGARGV | 147 | HLA-A02:01 | 16 |
| LVVVGARGV | 146 | HLA-B55:01 | 16 |
| YKLVVVGAR | 148 | HLA-A33:03 | 16 |
| KLVVVGAR | 149 | HLA-A74:01 | 17 |
| KLVVVGARGV | 147 | HLA-B13:02 | 17 |
| RGVGKSAL | 144 | HLA-C01:02 | 17 |
| LVVVGARGV | 146 | HLA-A68:02 | 18 |
| VVVGARGV | 150 | HLA-B55:01 | 18 |
| ARGVGKSAL | 142 | HLA-B15:09 | 19 |
| ARGVGKSAL | 142 | HLA-C14:03 | 20 |
| GARGVGKSA | 143 | HLA-B54:01 | 20 |
| VVVGARGV | 150 | HLA-B52:01 | 20 |

In some embodiments, a peptide comprises a RAS G12S mutation according to Table 10 below.

TABLE 10

| | SEQ | | Rank of |
|---|---|---|---|
| Peptide | ID NO: | Allele | Binding Potential |
| VVVGASGVGK | 151 | HLA-A11:01 | 1 |
| VVGASGVGK | 152 | HLA-A11:01 | 2 |
| VVGASGVGK | 152 | HLA-A03:01 | 3 |
| VVVGASGVGK | 151 | HLA-A68:01 | 4 |
| ASGVGKSAL | 153 | HLA-C03:04 | 5 |
| ASGVGKSAL | 153 | HLA-B46:01 | 5 |
| VVGASGVGK | 152 | HLA-A68:01 | 6 |
| VVVGASGVGK | 151 | HLA-A03:01 | 6 |
| ASGVGKSAL | 153 | HLA-C01:02 | 7 |
| GASGVGKSAL | 154 | HLA-B48:01 | 7 |
| ASGVGKSAL | 153 | HLA-C07:01 | 8 |
| ASGVGKSAL | 153 | HLA-C08:02 | 9 |
| GASGVGKSAL | 154 | HLA-B81:01 | 9 |
| SGVGKSAL | 155 | HLA-B08:01 | 9 |
| ASGVGKSAL | 153 | HLA-C03:03 | 10 |
| ASGVGKSAL | 153 | HLA-C03:02 | 10 |
| SGVGKSAL | 155 | HLA-B14:02 | 10 |
| VVGASGVGK | 152 | HLA-A30:01 | 10 |
| ASGVGKSAL | 153 | HLA-C08:01 | 11 |
| VVVGASGVGK | 151 | HLA-A30:01 | 11 |
| GASGVGKSAL | 154 | HLA-B35:03 | 12 |
| SGVGKSAL | 155 | HLA-C07:01 | 12 |
| ASGVGKSAL | 153 | HLA-B81:01 | 13 |
| GASGVGKSA | 156 | HLA-B55:01 | 13 |
| GASGVGKSAL | 154 | HLA-C03:03 | 13 |
| KLVVVGASGV | 157 | HLA-A02:01 | 13 |
| LVVVGASGV | 158 | HLA-A68:02 | 13 |
| SGVGKSAL | 155 | HLA-C01:02 | 13 |
| ASGVGKSA | 159 | HLA-B46:01 | 14 |
| ASGVGKSAL | 153 | HLA-C15:02 | 14 |
| GASGVGKSAL | 154 | HLA-C08:01 | 15 |
| SGVGKSAL | 155 | HLA-C03:04 | 15 |
| ASGVGKSAL | 153 | HLA-C05:01 | 16 |
| GASGVGKSAL | 154 | HLA-C03:04 | 16 |
| VVGASGVGK | 152 | HLA-A74:01 | 16 |
| ASGVGKSAL | 153 | HLA-B48:01 | 17 |
| GASGVGKSAL | 154 | HLA-C01:02 | 17 |
| SGVGKSAL | 155 | HLA-C03:02 | 17 |
| SGVGKSALTI | 160 | HLA-A23:01 | 17 |
| VGASGVGKSA | 161 | HLA-B55:01 | 18 |
| ASGVGKSAL | 153 | HLA-C12:03 | 19 |
| ASGVGKSAL | 153 | HLA-B57:03 | 19 |
| KLVVVGASGV | 157 | HLA-A02:07 | 19 |
| SGVGKSAL | 155 | HLA-B81:01 | 19 |
| ASGVGKSAL | 153 | HLA-C17:01 | 20 |
| KLVVVGASG | 162 | HLA-A32:01 | 20 |

In some embodiments, a peptide comprises a RAS G12V mutation according to Table 11 below.

TABLE 11

| | SEQ | | Rank of |
|---|---|---|---|
| Peptide | ID NO: | Allele | Binding Potential |
| VVGAVGVGK | 19 | HLA-A03:01 | 1 |
| VVGAVGVGK | 19 | HLA-A11:01 | 2 |
| VVVGAVGVGK | 20 | HLA-A11:01 | 2 |
| VVVGAVGVGK | 20 | HLA-A68:01 | 3 |
| VVGAVGVGK | 19 | HLA-A68:01 | 4 |
| LVVVGAVGV | 163 | HLA-A68:02 | 5 |
| VVGAVGVGK | 19 | HLA-A30:01 | 5 |
| AVGVGKSAL | 16 | HLA-B81:01 | 6 |
| KLVVVGAVGV | 164 | HLA-A02:01 | 6 |
| AVGVGKSAL | 16 | HLA-B46:01 | 7 |
| GAVGVGKSAL | 18 | HLA-C03:03 | 7 |
| GAVGVGKSAL | 18 | HLA-B48:01 | 7 |
| VVVGAVGVGK | 20 | HLA-A03:01 | 7 |
| AVGVGKSAL | 16 | HLA-C03:04 | 8 |
| GAVGVGKSAL | 18 | HLA-C03:04 | 8 |
| KLVVVGAVGV | 164 | HLA-A02:07 | 9 |
| VGVGKSAL | 165 | HLA-B08:01 | 9 |
| VVVGAVGV | 166 | HLA-A68:02 | 9 |
| AVGVGKSAL | 16 | HLA-C08:02 | 10 |
| AVGVGKSAL | 16 | HLA-B07:02 | 10 |
| GAVGVGKSAL | 18 | HLA-B35:03 | 10 |
| AVGVGKSAL | 16 | HLA-C08:01 | 11 |
| AVGVGKSAL | 16 | HLA-C01:02 | 11 |
| GAVGVGKSA | 17 | HLA-B55:01 | 11 |
| GAVGVGKSAL | 18 | HLA-B81:01 | 11 |
| GAVGVGKSAL | 18 | HLA-C08:01 | 11 |
| KLVVVGAVGV | 164 | HLA-B13:02 | 11 |
| VGVGKSAL | 165 | HLA-C03:04 | 11 |
| AVGVGKSAL | 16 | HLA-A32:01 | 12 |
| GAVGVGKSA | 17 | HLA-B46:01 | 12 |
| VGVGKSAL | 165 | HLA-C03:02 | 12 |
| VGVGKSALTI | 167 | HLA-A23:01 | 12 |
| GAVGVGKSA | 17 | HLA-B54:01 | 13 |
| VGVGKSAL | 165 | HLA-C01:02 | .3 |
| AVGVGKSAL | 16 | HLA-B48:01 | 14 |
| AVGVGKSAL | 16 | HLA-C03:03 | 14 |
| AVGVGKSAL | 16 | HLA-B42:01 | 14 |
| LVVVGAVGV | 163 | HLA-B55:01 | 14 |
| VGVGKSAL | 165 | HLA-C08:01 | 14 |
| VVGAVGVGK | 19 | HLA-A74:01 | 14 |
| AVGVGKSAL | 16 | HLA-C05:01 | 15 |
| AVGVGKSAL | 16 | HLA-C03:02 | 15 |
| GAVGVGKSA | 17 | HLA-C03:04 | 15 |
| KLVVVGAVGV | 164 | HLA-A02:04 | 15 |
| LVVVGAVGV | 163 | HLA-A02:07 | 15 |
| VGVGKSAL | 165 | HLA-B14:02 | 15 |
| VVVGAVGVGK | 20 | HLA-A30:01 | 15 |
| VVGAVGVGK | 19 | HLA-B81:01 | 16 |
| VVVGAVGV | 166 | HLA-B55:01 | 16 |
| AVGVGKSAL | 16 | HLA-C14:03 | 17 |
| AVGVGKSAL | 16 | HLA-B15:01 | 17 |
| LVVVGAVGV | 163 | HLA-B54:01 | 17 |
| AVGVGKSA | 168 | HLA-B55:01 | 18 |
| AVGVGKSAL | 16 | HLA-C17:01 | 18 |
| GAVGVGKSA | 17 | HLA-B50:01 | 19 |
| GAVGVGKSAL | 18 | HLA-C17:01 | 19 |
| YKLVVVGAV | 169 | HLA-A02:04 | 19 |
| GAVGVGKSAL | 18 | HLA-B35:01 | 20 |
| VVGAVGVGK | 19 | HLA-A31:01 | 20 |
| YKLVVVGAV | 169 | HLA-B51:01 | 20 |

In some embodiments, a peptide comprises a RAS G13C mutation according to Table 12 below.

TABLE 12

| | SEQ | | Rank of |
|---|---|---|---|
| Peptide | ID NO: | Allele | Binding Potential |
| VVVGAGCVGK | 170 | HLA-A11:01 | 1 |
| VVGAGCVGK | 171 | HLA-A11:01 | 2 |
| AGCVGKSAL | 172 | HLA-C01:02 | 3 |
| VVGAGCVGK | 171 | HLA-A03:01 | 4 |
| VVVGAGCVGK | 170 | HLA-A68:01 | 4 |
| CVGKSALTI | 173 | HLA-B13:02 | 5 |
| VVGAGCVGK | 171 | HLA-A68:01 | 5 |
| VVGAGCVGK | 171 | HLA-A30:01 | 6 |
| AGCVGKSAL | 172 | HLA-B48:01 | 7 |
| AGCVGKSAL | 172 | HLA-C03:04 | 8 |
| GCVGKSALTI | 174 | HLA-B49:01 | 8 |
| AGCVGKSAL | 172 | HLA-C08:02 | 9 |
| VVVGAGCVGK | 170 | HLA-A03:01 | 9 |
| KLVVVGAGC | 175 | HLA-A30:02 | 10 |
| GCVGKSAL | 176 | HLA-C07:01 | 11 |
| VVGAGCVGK | 171 | HLA-A74:01 | 12 |
| AGCVGKSAL | 172 | HLA-C14:03 | 13 |
| KLVVVGAGC | 175 | HLA-B15:01 | 14 |

In some embodiments, a peptide comprises a RAS G13D mutation according to Table 13 below.

TABLE 13

| | SEQ | | Rank of |
|---|---|---|---|
| Peptide | ID NO: | Allele | Binding Potential |
| AGDVGKSAL | 177 | HLA-C08:02 | 1 |
| AGDVGKSAL | 177 | HLA-C05:01 | 2 |
| VVGAGDVGK | 178 | HLA-A11:01 | 3 |
| VVVGAGDVGK | 179 | HLA-A11:01 | 3 |
| VVVGAGDVGK | 179 | HLA-A68:01 | 4 |
| GAGDVGKSA | 180 | HLA-B46:01 | 5 |
| GAGDVGKSAL | 181 | HLA-B48:01 | 5 |
| VVGAGDVGK | 178 | HLA-A68:01 | 5 |
| VVGAGDVGK | 178 | HLA-A03:01 | 5 |
| AGDVGKSAL | 177 | HLA-C03:04 | 6 |
| AGDVGKSAL | 177 | HLA-C04:01 | 6 |
| AGDVGKSAL | 177 | HLA-C01:02 | 6 |
| DVGKSALTI | 182 | HLA-B13:02 | 6 |
| DVGKSALTI | 182 | HLA-A25:01 | 6 |
| GDVGKSAL | 183 | HLA-C07:01 | 6 |
| GDVGKSAL | 183 | HLA-B40:02 | 7 |
| GDVGKSAL | 183 | HLA-B37:01 | 8 |
| AGDVGKSAL | 177 | HLA-B48:01 | 9 |
| DVGKSALTI | 182 | HLA-B51:01 | 10 |
| VVGAGDVGK | 178 | HLA-A30:01 | 10 |
| GAGDVGKSAL | 181 | HLA-C08:01 | 11 |
| GAGDVGKSAL | 181 | HLA-B81:01 | 11 |
| AGDVGKSAL | 177 | HLA-C08:01 | 12 |
| GAGDVGKSAL | 181 | HLA-C03:04 | 12 |
| DVGKSALTI | 182 | HLA-B53:01 | 13 |
| AGDVGKSAL | 177 | HLA-B07:02 | 14 |
| AGDVGKSAL | 177 | HLA-B46:01 | 14 |
| DVGKSALTI | 182 | HLA-A26:01 | 14 |
| VVGAGDVGK | 178 | HLA-A74:01 | 14 |
| GAGDVGKSA | 180 | HLA-B54:01 | 15 |
| DVGKSALTI | 182 | HLA-B38:01 | 16 |
| GAGDVGKSAL | 181 | HLA-C03:03 | 16 |
| VVVGAGDVGK | 179 | HLA-A03:01 | 16 |

In some embodiments, a peptide comprises a mutated RAS peptide according to Table 14 below.

TABLE 14

| | SEQ | MHC protein encoded |
|---|---|---|
| Epitope | ID NO: | by allele |
| GACGVGKSA | 128 | C03:04 |
| GAVGVGKSA | 17 | C03:03 |

### Example 3. Method for Enrichment and Expansion of Antigen-Specific T Cells

In this example, a method for increasing the yield of antigen specific T cells is described. An overview of an examplary manufacturing process used to produce large number of KRAS neoantigen-specific T cells is presented in FIG. 1A, FIG. 1B and FIG. 2. In brief, peripheral blood mononuclear cells (PBMCs) derived from leukaphereses from healthy donors or patients were processed to deplete specific cell subsets on day 0. KRAS neoantigen peptides were added on day 1 to the cell culture, and maturation cytokines were added after a period of time. On day 0, human serum was added to the culture. On days 5, 7, 9 and 12, culture maintenance was performed, which entailed addition or changing of media and/or addition of cytokines. On day 13, the KRAS neoantigen peptides were spiked into the culture, which upregulated activation markers on the T cells. On day 14, antigen-specific T cells were enriched by antibody labeling and capturing of the cells expressing one or more of the activation markers. The process on days 13 and 14 are termed the “enrichment process.”

### Enrichment of Antigen Specific T Cells

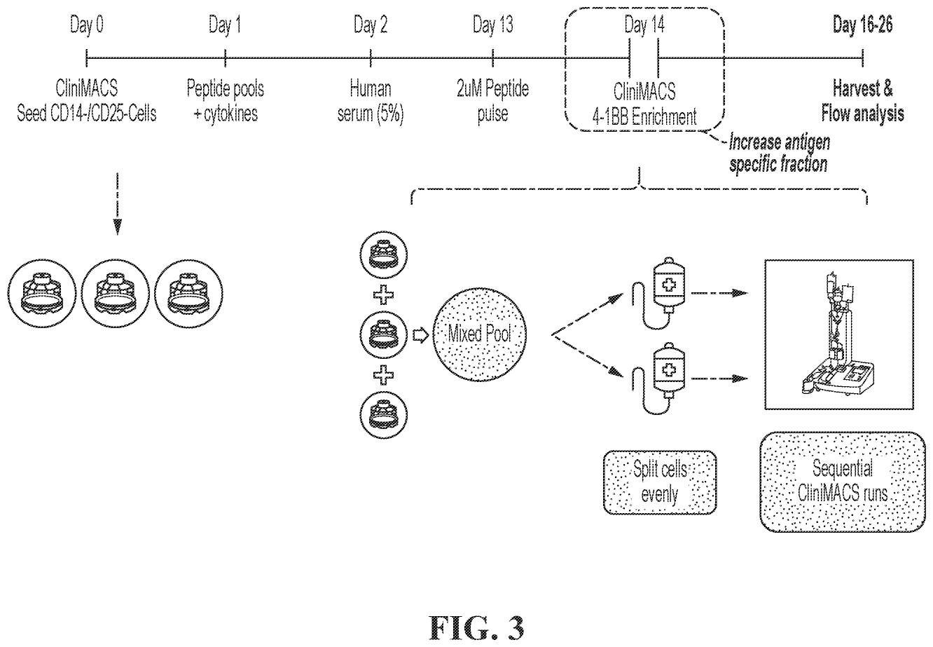

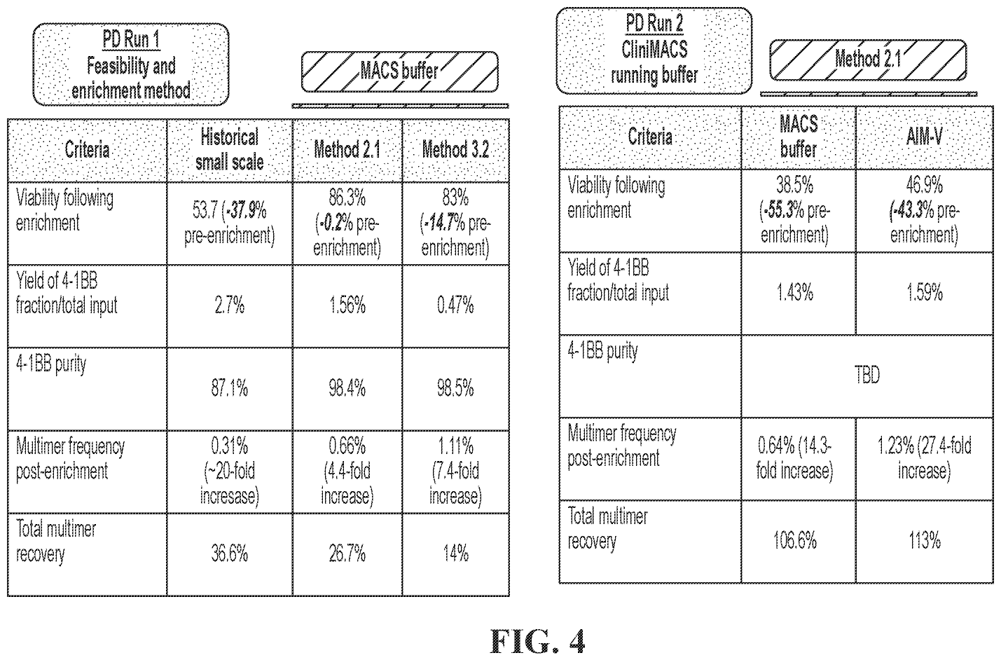

FIGS. 3 and 4 demonstrate the schematic workflow and results from the feasibility of enrichment at research scale and large-scale runs using the CliniMACS system. For large scale runs, at least 10\hat{ }9 cells were available to set up the runs. The naïve T cell induction protocol described above is used in large vessels, and the cells are pooled on day 14 after peptide pulsing and were run through the CliniMACS. FIG. 4 shows results from the studies on feasibility of enrichment. Both the methods tested using MACS buffer yielded viable cells after enrichment which was considerably higher over historical small scale data. Multiple CliniMACS protcols were tested, which have different pressures and flow rates through the magnetic column (e.g., manufacturer specified protocols named Method 2.1, Method 3.2, etc.), and the enrichment was relatively similar Method 2.1 was selected for further testing. Different running buffers were tested for passing through the CliniMACS columns, including MACS buffer and AIM-V, and the results were relatively similar.

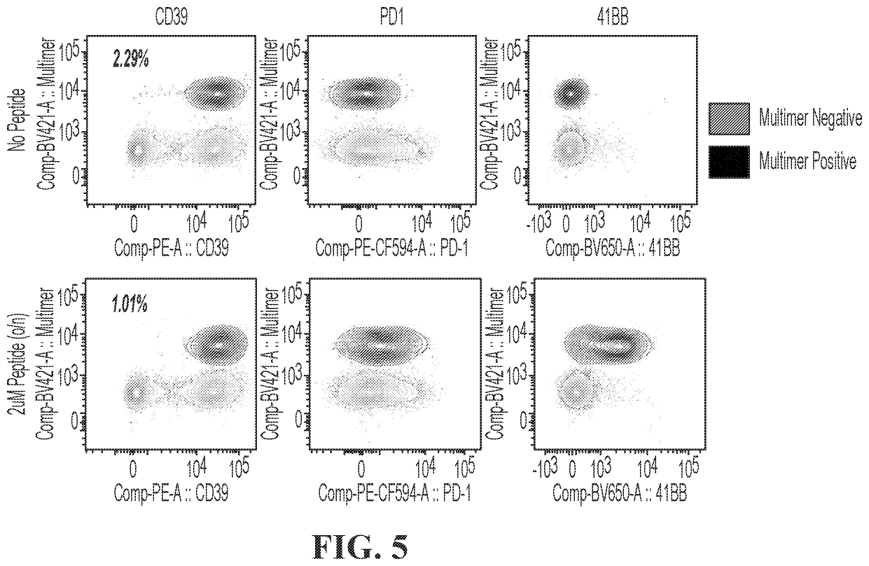

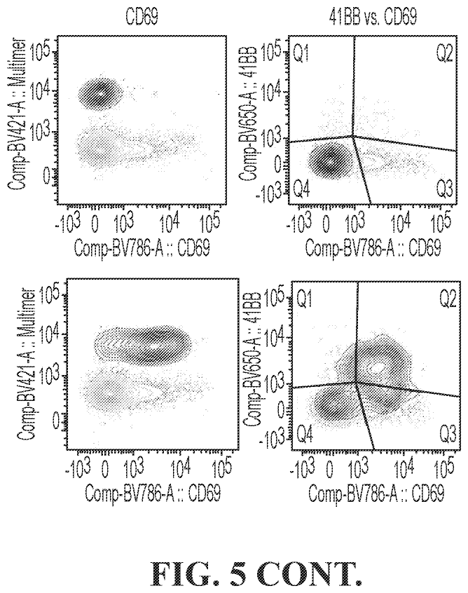

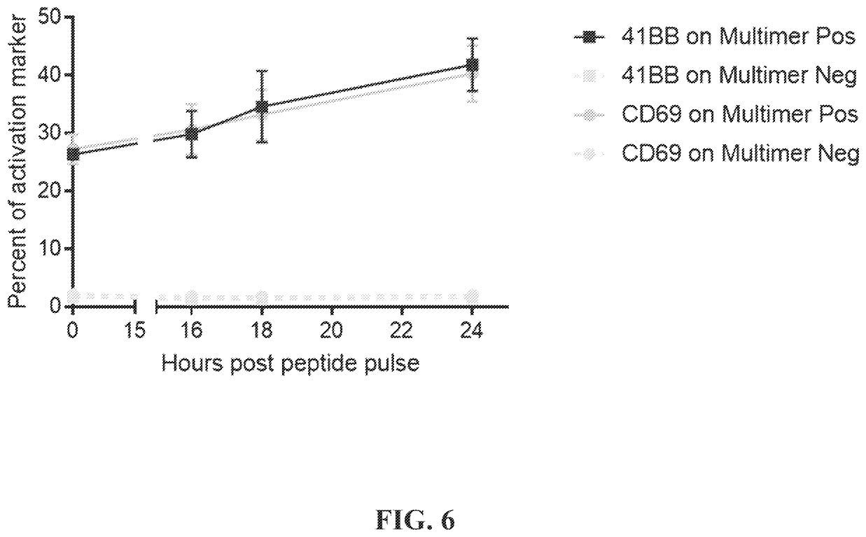

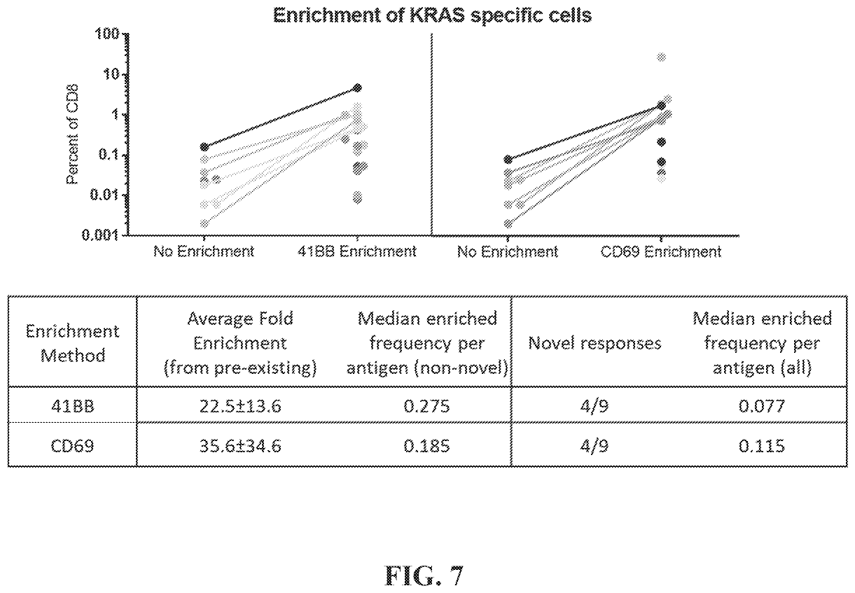

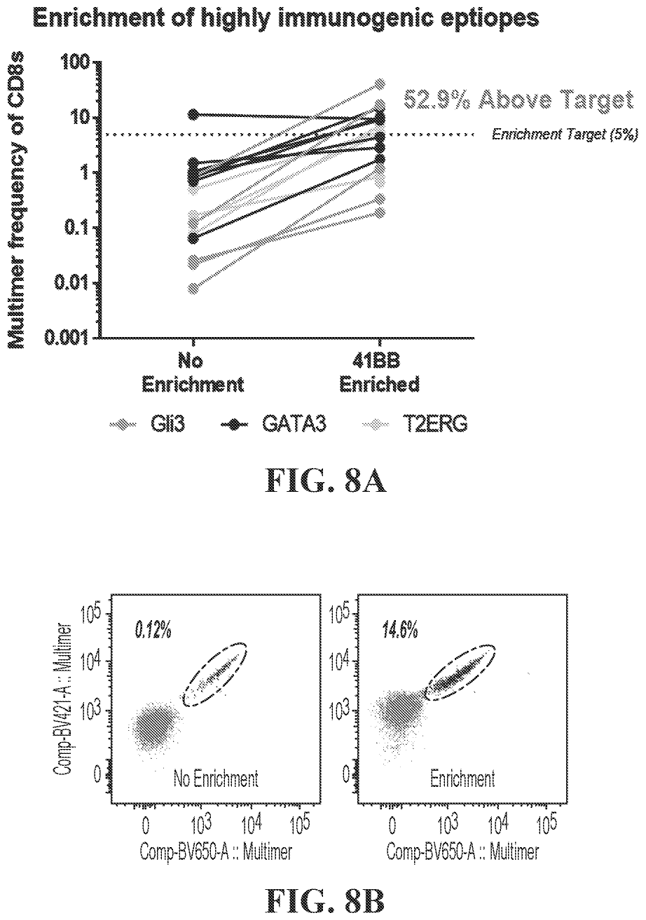

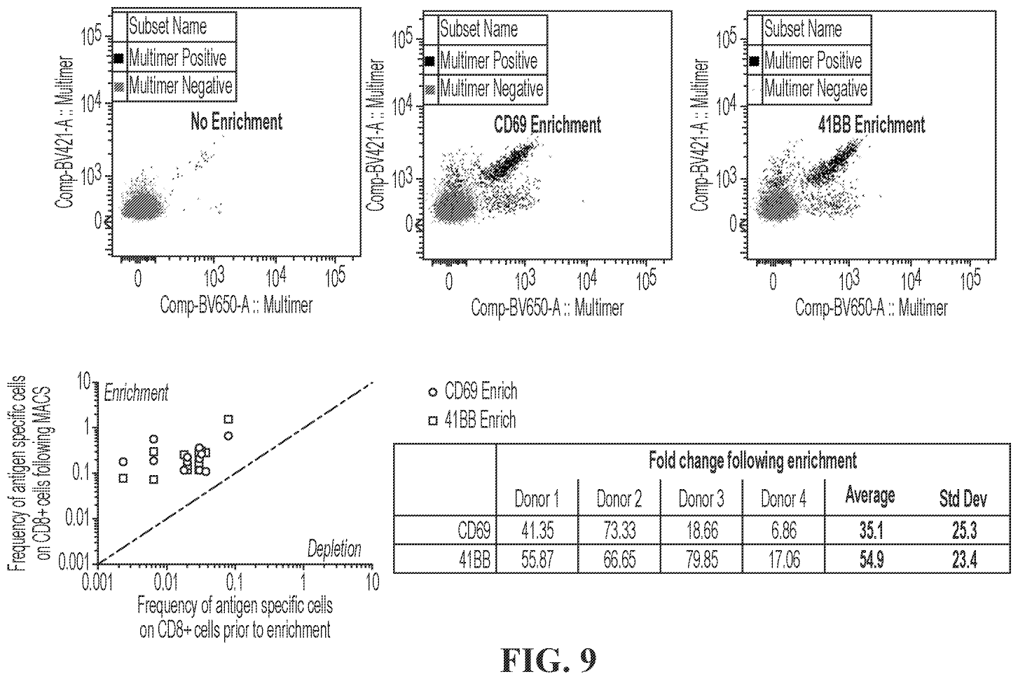

After the initial stimulation of naïve T cells, the peptide pulse on day 13 can be used to, for example, upregulate cell surface activation markers that can be used to label and enrich antigen-specific T cells. Alternative methods, such as addition of antigen-expressing cells or scaffolds can function in a similar manner. FIG. 5 shows the upregulation of four different cell surface activation markers (CD39, PD-1, CD137 (4-1BB), and CD69) after peptide pulse on day 13 and analysis on day 14. FIGS. 5 and 6 demonstrates data that an overnight peptide pulse (up to 24 hours) upregulates specific activation markers on antigen responsive T cells. Of the activation markers shown, 4-1BB (CD137) and CD69 showed great promise (FIG. 6). CD137 and CD69 were more substantially increased on multimer positive cells relative to multimer negative cells. Particularly, as shown in FIG. 5, CD137 enrichment and peptide spike with 2 uM peptide had a remarkable effect on enriching antigen-specific (multimer positive) T cells (data from lower right compared to the upper right flow cytometry results), exceeding that of CD69. FIG. 7 shows that KRAS neoantigen-specific CD8+ T cells were enriched using either CD137 or CD69 using this method. Pre-enrichment frequencies were increased on average greater than 10-fold and antigen-specific responses that were not detectable prior to enrichment became detectable after enrichment (novel responses). These results show that this method can be applied broadly and can further increase the antigen-specific frequency of relatively highly immunogenic epitopes (FIGS. 8A and 8B). FIG. 9 provides a further comparison between CD69 and CD137 enrichment on antigen-specific CD8+ T cells. CD137 enrichment led on an average to a higher fold change of antigen-specific CD8+ T cells relative to CD69 enrichment.

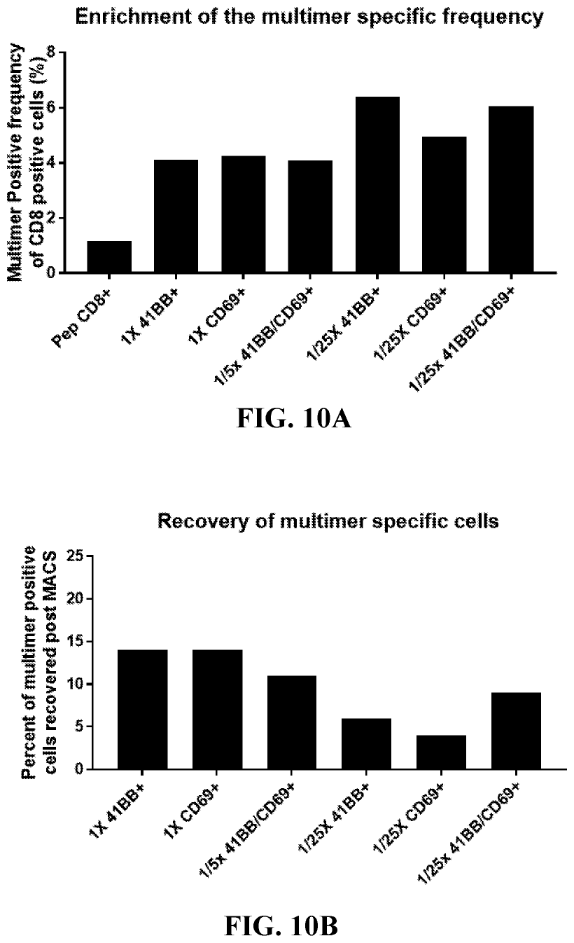

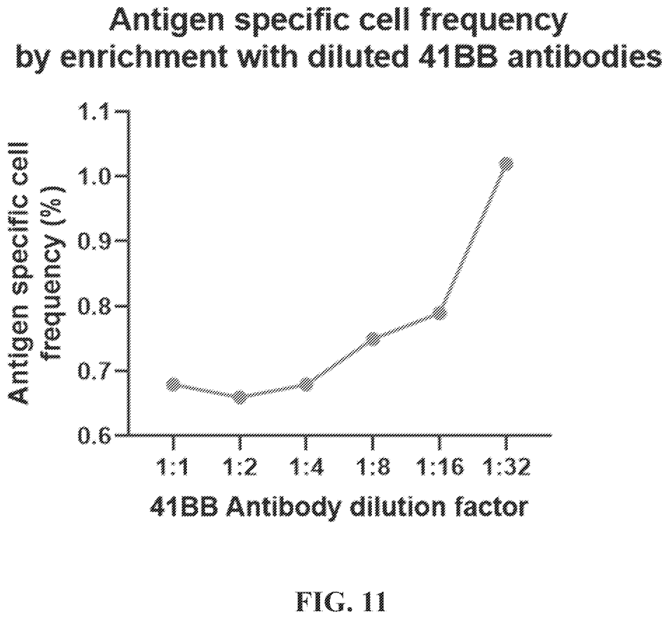

FIGS. 10A, 10B and 11 exhibits improvement in the enrichment process that led to the appreciable success of the protocol in enriching antigen-specific T cells. It was observed that further titrating and diluting the antibodies concentrations for enriching the antigen specific T cells could lead to increased yield of the multimer positive cells. FIGS. 10A and 10B show that enrichment with decreasing amounts of antibodies against CD137 or CD69 led to an increase in the fraction of enriched cells that are antigen-specific, potentially due to the high surface expression of these markers relative to multimer negative cells as shown in FIG. 5.

### Expansion of Antigen Specific T Cells

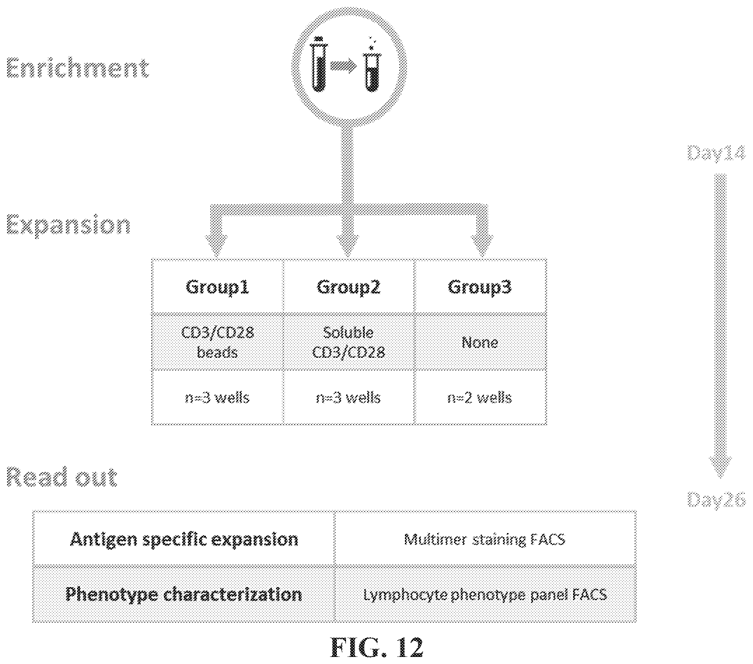

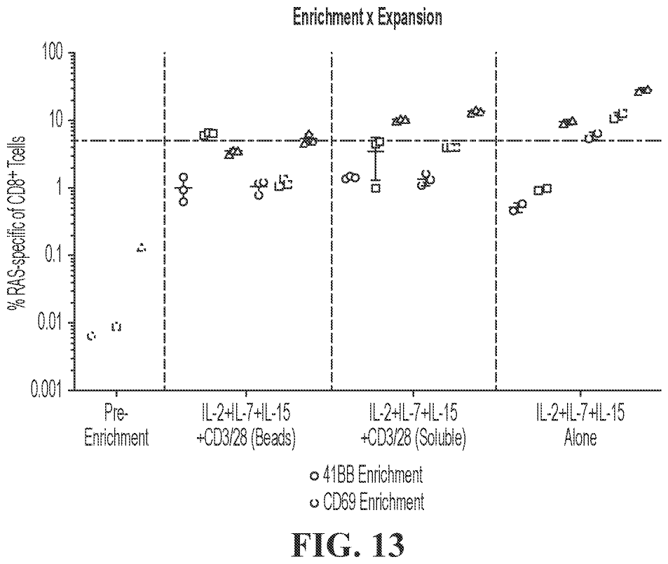

FIG. 12 shows a schematic representation of a study for improvement of expansion of antigen specific (i.e. antigen restricted) T cells by using T cell activator in the culture medium. Following enrichment of the cells, at day 14, one group (Group 1) of cells were cultured in presence of CD3/CD28 coated beads, Another group (Group 2) were cultured in the presence of soluble CD3/CD28 in the culture medium. Group 3 was control. Cells were then taken through an expansion process, which can have variable steps. On day 16, 19, 21, and 23 culture maintenance was performed as described above. Additional reagents can be added into the culture relative to the initial stimulation, and on additional days. On day 26, cells were harvested and formulated. Cells were tested at least for antigen specificity (multimer assay, staining and flow cytometry), and phenotyping of lymphocytes using activated lymphocyte-specific marker panel by flow cytometry. Results obtained from this process and data supporting the feasibility of the expansion process are shown in FIGS. 13, 14A, 14B, 15, 16, 17A, 17B, 18, 19, 20 and 21.

FIG. 13 shows data from a study represented graphically in FIG. 12, where KRAS neoantigen-specific T cells were enriched using either CD137 or CD69 from three healthy donors, and then subjected to one of three expansion protocols. These data demonstrate that the expansion method can perform differently for different enrichment methods. In general, soluble CD3/CD28 showed higher potential to expand RAS-specific CD8+ T cells.

### Effect of an Exponential Peptide Pulse on Cell Expansion

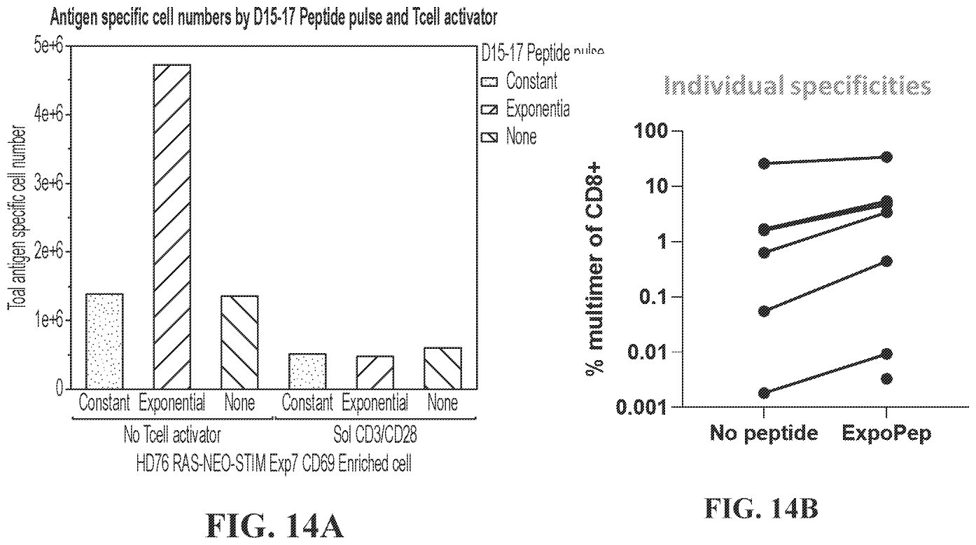

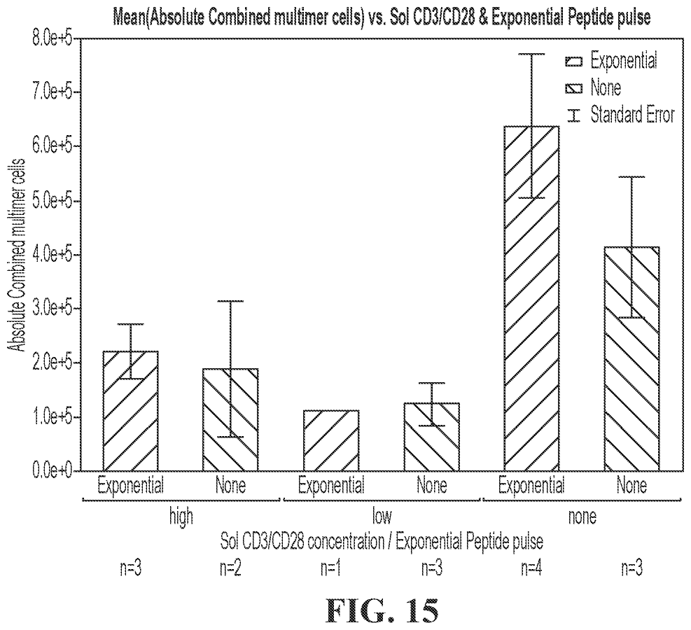

It was further observed that the expansion of antigen restricted T cells can be improved by adding peptide antigen pulses during the expansion phase. During the expansion protocol, cells can be further exposed to the antigen to provide additional stimulation for the antigen-specific T cells. Additionally, the peptide amount can be gradually increased by 2-fold or more in each pulse, which is loosely termed in this context, an exponential peptide pulse. FIG. 14A demonstrates that exposure to increasing amounts of antigen can substantially increase the number of antigen-specific T cells after CD69 enrichment. FIG. 14B demonstrates that this occurs for all specificities within a single culture. FIG. 15 demonstrates that this process works after CD137 enrichment as well, and specifically works optimally in the absence of anti-CD3/anti-CD28. This may be because the anti-CD3 provides a strong TCR signal for all T cells as opposed to peptide stimulation, which preferentially leads to stimulation of antigen-specific T cells that recognize the peptide.

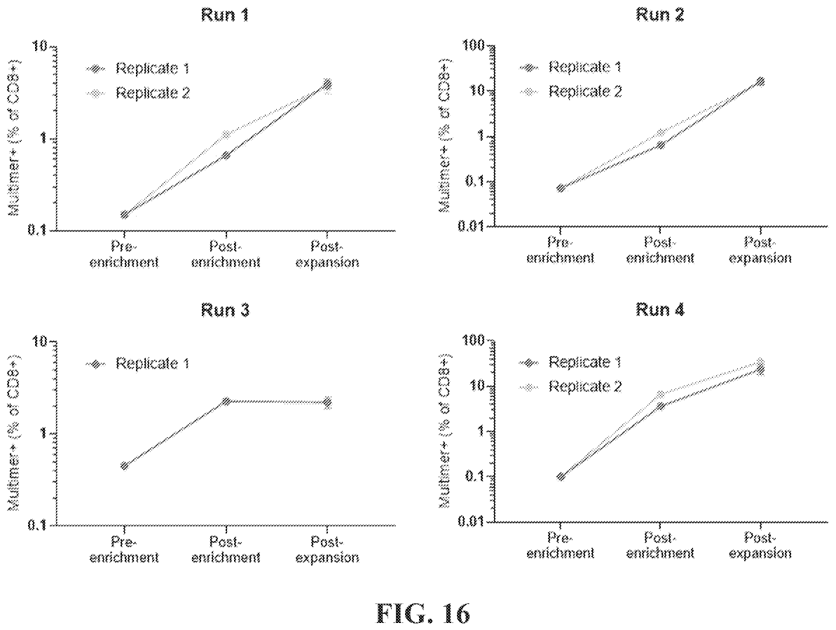

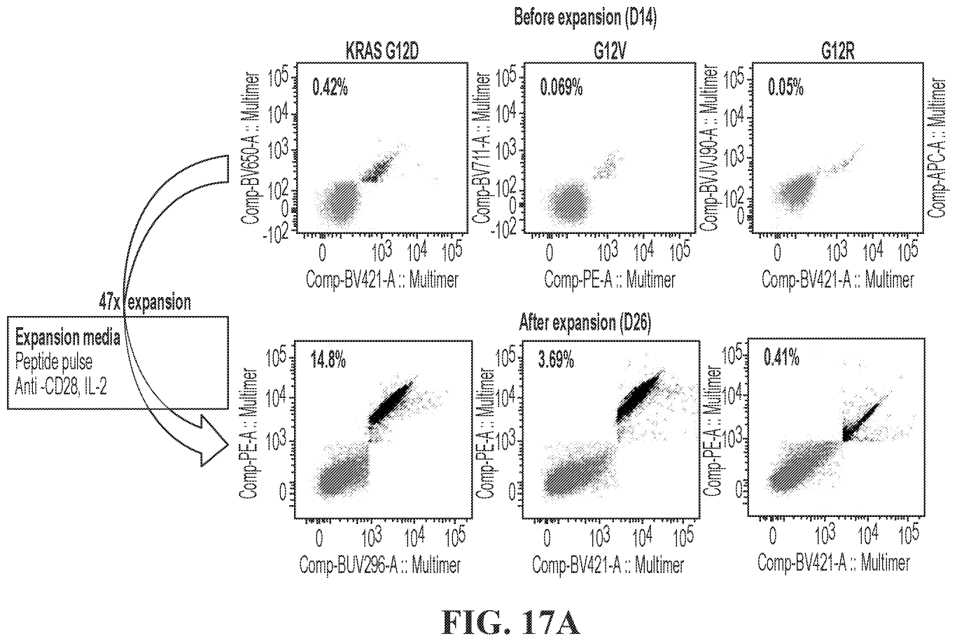

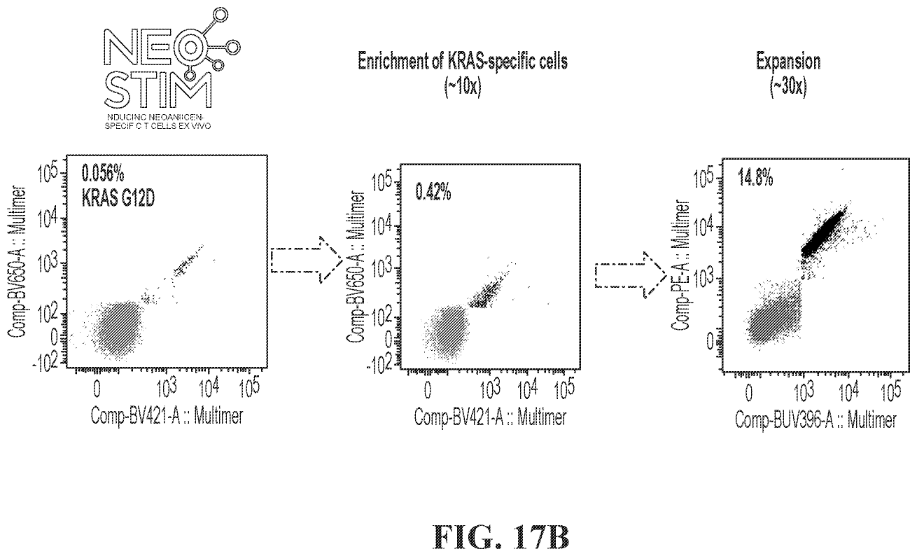

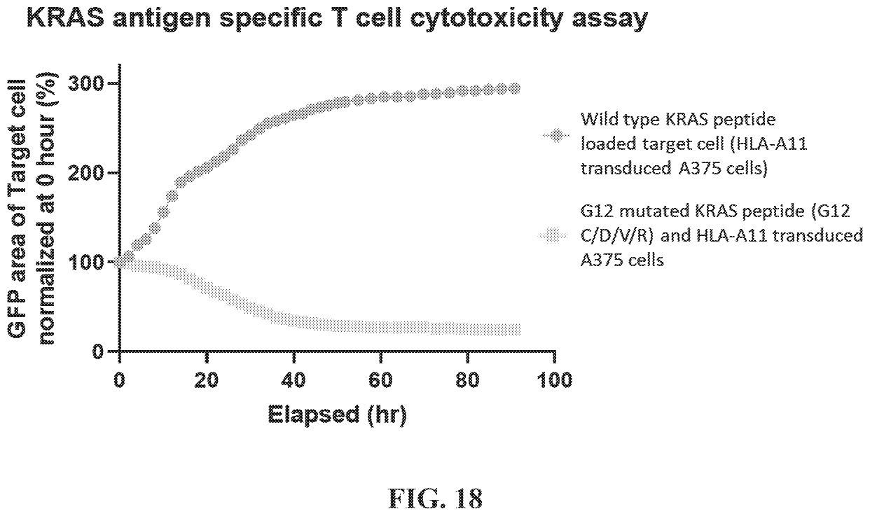

The combination of the enrichment and expansion can dramatically increase the fraction of antigen-specific T cells. FIG. 16 shows that the antigen-specific frequency of CD8+ T cells in culture can increase by 1-2 orders of magnitude. FIGS. 17A and 17B shows that this occurred concurrently for multiple specificities within a single culture. FIG. 18 shows that the cells expanded during this process were highly functional and retained specificity for mutant over wild-type epitopes.

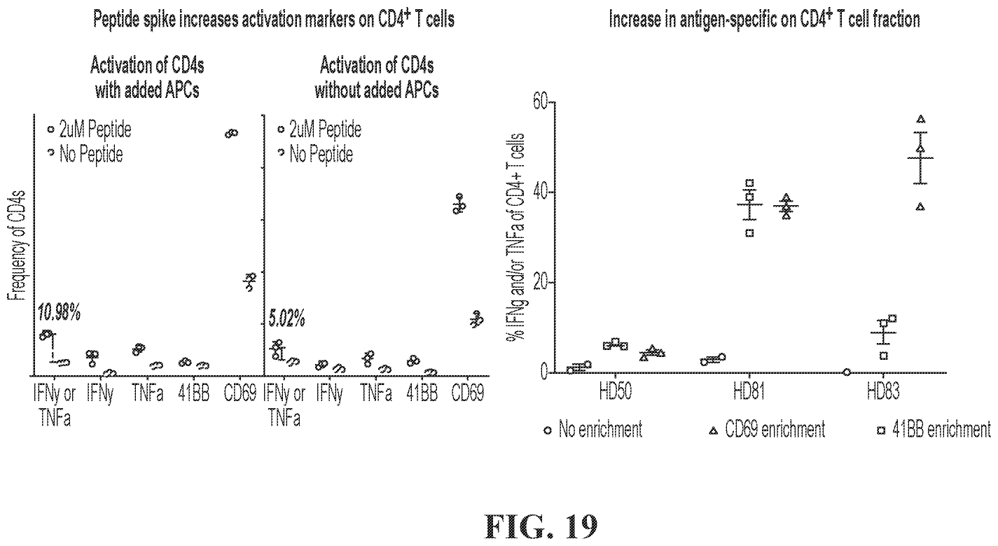

FIG. 19 shows that the enrichment protocol leads to enrichment and expansion of antigen specific CD4+ T cells, and this was validated in studies with cells from multiple donors.

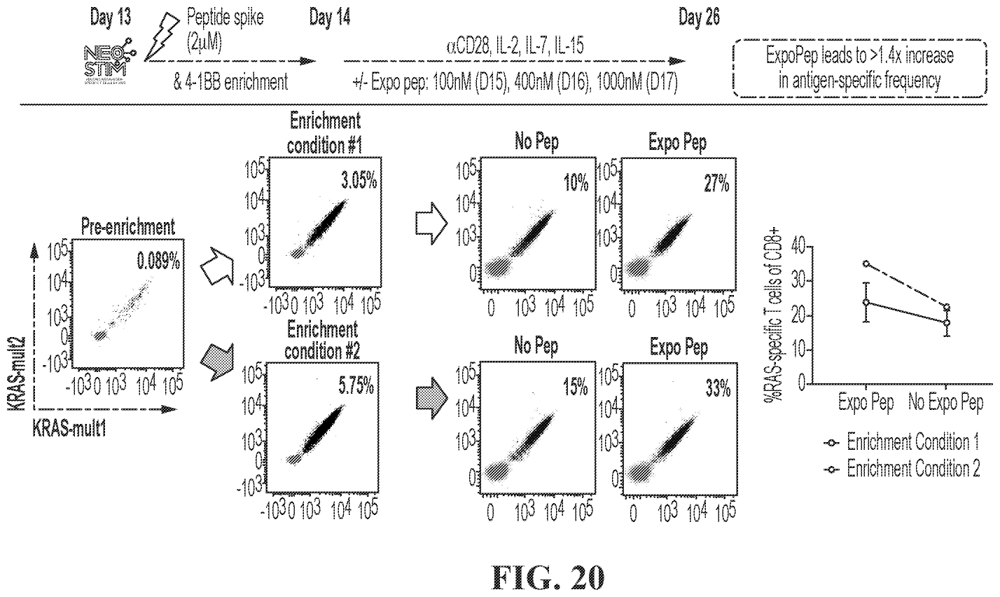

In another development for improved antigen-specific T cell expansion, cells were subjected to a very low concentration of the peptide antigen after enrichment, followed by exponential increase of the pulse peptide concentration. FIG. 20 top panel summarizes a workflow, wherein T cells were induced and cultured for 13 days as indicated in FIG. 1B or FIG. 2, then subjecting the cells to an overnight (or up to 24 hours) antigen spike by adding peptide antigen at 2 uM concentration, followed by CD137 enrichment on day 14. On day 15, cells were stimulated with 100 nM peptide antigen. On day 16, cells were stimulated with 400 nM peptide concentration, and on day 17, with 1000 nM peptide concentration (exponential peptide stimulation). Results shown in FIG. 20 bottom panel indicate that exponential peptide stimulation led to 1.4 fold increase in antigen specific T cell stimulation compared to no peptide.

Exponential Peptide Pulse LED to Expansion of T Cells Having TCRs with High Avidity

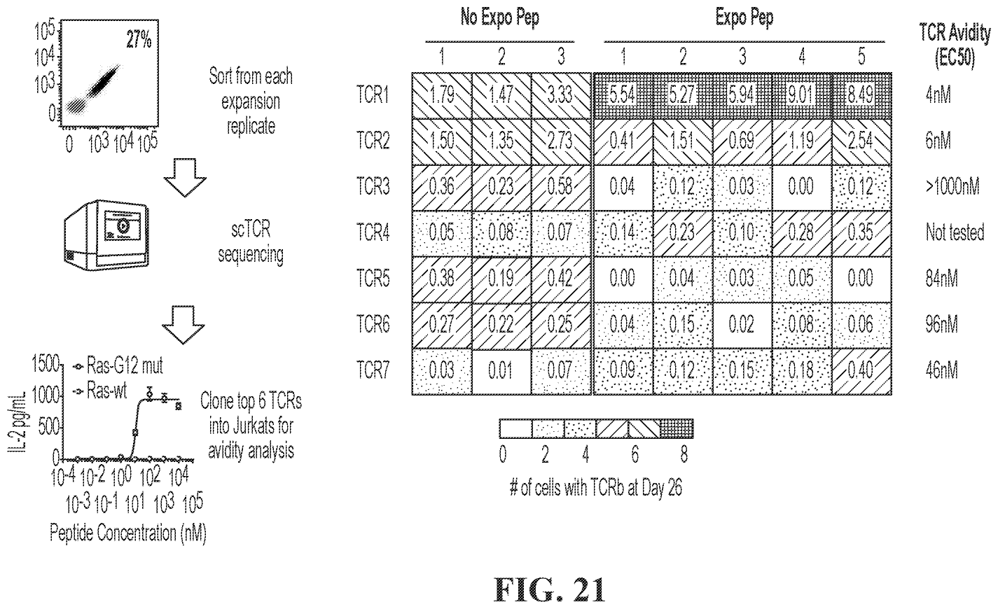

T cells expanded using the methods described above were sorted for expansion with each antigen and the TCR expressed in the cells were sequenced. Top 6 TCRs were cloned into Jurkat cells and tested for avidity (FIG. 21 left panel). A clear improvement was shown in expanding T cells having TCRs with high avidity when exponential peptide pulse was used (FIG. 21 right panel). The TCRs were robust and with very high avidity with the exponential peptide pulse.

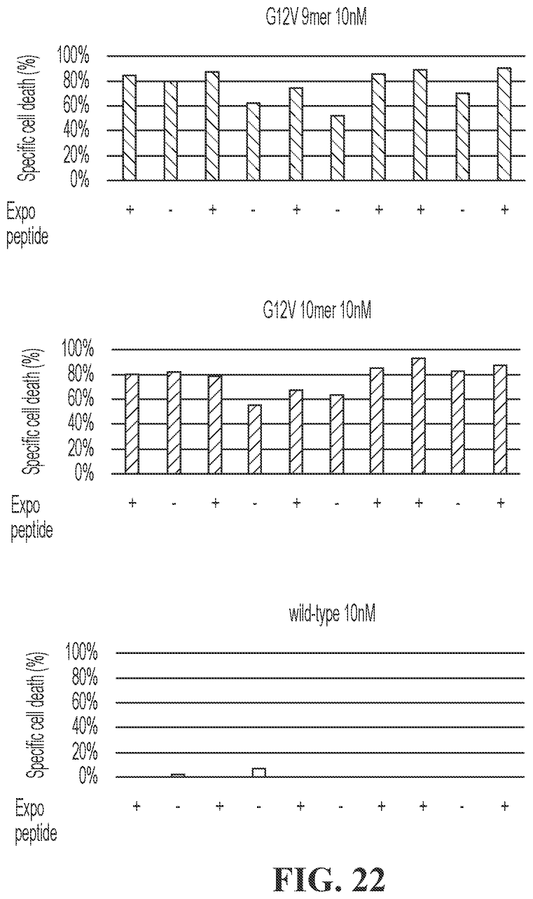

Further, FIG. 22 shows that exponential peptide pulse led to T cells that retained higher target specific cytotoxicity compared to no peptide pulse.

### Expansion Protocol Alone can Increase Antigen-Specific T Cells Independent of the Enrichment Step

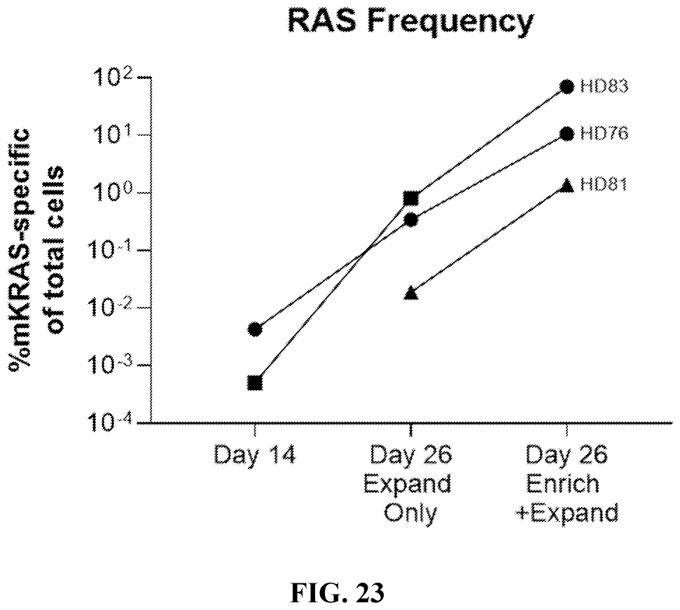

This study was performed to see if both enrichment and expansion are necessary in increasing antigen specific T cells. Sets of cells from multiple donors (e.g. HD81, HD83 and HD76) were each divided into two sets, one set enriched and expanded with mutated KRAS antigens, and the other only expanded with the same antigens using the same expansion protocol (described above) without the enrichment step. In absence of the enrichment step, all cells expanded by about 1-3 orders of magnitude. FIG. 23 shows that the expansion protocol alone (no enrichment) increases the frequency of antigen-specific T cells at day 26 relative to day 14, but that the expansion protocol with enrichment together further increases the frequency of antigen-specific T cells at day 26 relative to day 14.

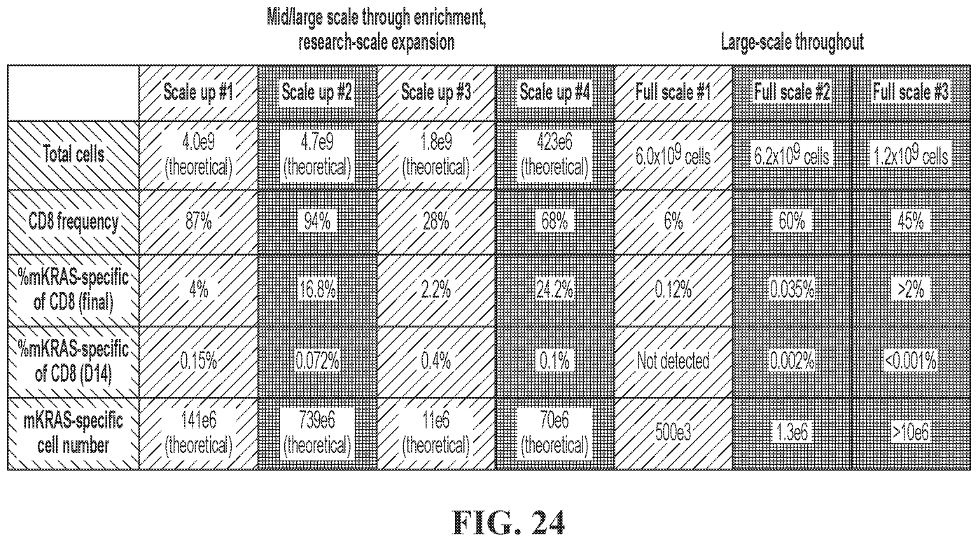

FIG. 24 further shows that the method described above was compatible for full scale expansion. Mid/large scale expansion was carried out at a scale similar to true manufacturing scale at the seeding stage, up to the enrichment stage, followed by research scale expansion procedure. In short approximately 2×10\hat{ }9 cells were seeded for mid-large scale culture. Research scale expansion was done for each conditions tested, using about 1×10\hat{ }6-5×10\hat{ }6 cells per assay condition. In case of large scale throughout, about 10\hat{ }9 cells were taken through the expansion procedure to obtain T cell therapeutic products. All cultures were performed in asceptic closed systems. With a starting population of between 6×10\hat{ }8 to 6×10″9 cells, the method led to successful generation and yield of antigen specific T cells. The yield of total number of cells and antigen specific T cells are in the range suitable for used as a therapeutic.

### NK Cells Large Scale Expansion in Antigen Specific T Cells

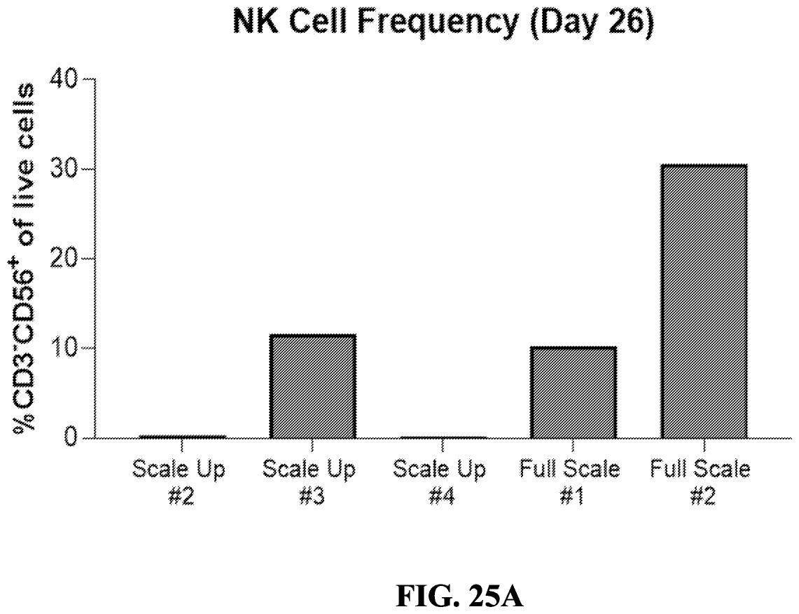

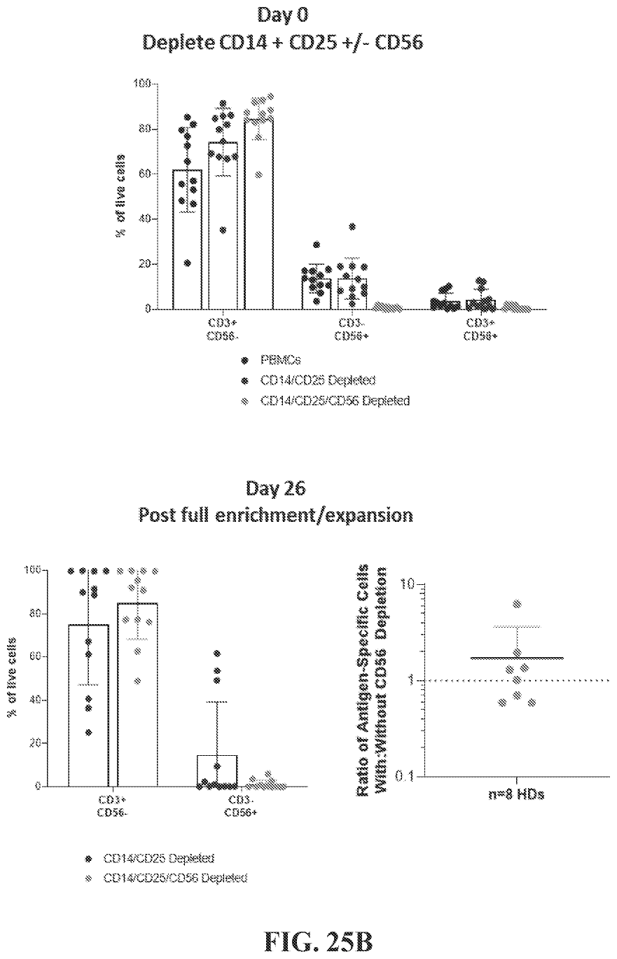

It was observed that large scale expansion of the T cells led to an expansion of NK cells that constituted to up to about 30% of the total live cell population at the end of expansion (day 26) (FIG. 25A). To address this issue, depletion of CD56 cells at the onset was attempted. CD56 depletion along with CD14 and CD25 depletion led to a depletion of CD56+NK cells, but also led to a decrease of CD3+/CD56+ cells initially (FIG. 25B left panel). However, at the end of the expansion phase, there was an increased T cell population in the NK cell depleted set, and an increase in antigen specific cells (FIG. 25B right panel). The protocol was modified for inclusion of CD56 cell depletion, along with CD14 and CD25 cell depletion at day 0.

A therapeutic composition of T cells can be generated using the method above, using GMP procedures and closed culture and harvesting conditions. The harvested cells can be stored under proper temperature and other conditions until infusion.

Care should be taken to maintain the functionality and antitumor activity of the cells upon infusion into a patient.

1-108. (canceled)

109. A method of producing an ex vivo population of expanded antigen specific T cells, the method comprising:
a) culturing T cells from a biological sample from a subject in a first cell culture medium comprising antigen presenting cells (APCs) to produce a first population of T cells, wherein the APCs present an epitope of a peptide antigen in complex with an WIC protein;
b) culturing the first population of T cells in a second cell culture medium to produce a second population of T cells;
c) enriching CD137 (4-1BB)-expressing T cells from the second population of T cells to produce a third population of T cells; and
d) expanding the third population of T cells in a third cell culture medium to obtain ex vivo population of expanded antigen specific T cells,
wherein, antigen specific T cells in the third population of T cells is at least ten-fold compared to antigen specific T cells in the first or the second population of T cells.

110. The method of claim 109, wherein enriching further comprises enriching CD69 expressing T cells from the second population of T cells to produce the third population of T cells.

111. The method of claim 110, wherein enriching comprises contacting with an enriching reagent comprising an anti-CD137 reagent and anti-CD69 reagent respectively.

112. The method of claim 109, wherein the biological sample is a sample of peripheral blood mononuclear cells (PBMCs) from the subject; and wherein the method further comprises depleting CD14+ cells from the PBMC prior to culturing.

113. The method of claim 109, wherein the method further comprises depleting CD25+ cells from the PBMC prior to culturing.

114. The method of claim 109, wherein the first cell culture medium comprises one or more peptide antigens comprising the peptide antigen; or wherein the APCs comprise a polynucleotide encoding the peptide antigen.

115. The method of claim 114, wherein the second culture medium is spiked with an amount of the peptide antigen such that a concentration of the peptide antigen in the first culture medium is that is at least the same as, or higher than a second concentration of the peptide antigen comprised in the second culture medium, wherein the first concentration of the peptide antigen or the second concentration of the peptide antigen ranges from 1 nM to 100 μM or 100 nM to 10 μM.

116. The method of claim 113, wherein the first concentration of the peptide antigen or the second concentration of the peptide antigen is 1 μM, 2 μM, 3 μM, 4 μM or 5 μM.

117. The method of claim 110, wherein the enriching CD137 (4-1BB)-expressing T cells and CD69 expressing T cells from the second population of T cells begins at least 10 days after beginning the culturing of the T cells from the biological sample from the subject in the first cell culture medium.

118. The method of claim 110, wherein expanding the third population of T cells in the third cell culture medium begins at least 1 day after enriching CD137 (4-1BB)-expressing T cells and CD69 expressing T cells.

119. The method of claim 109, wherein expanding the third population of T cells in the third cell culture medium comprises expanding the third population of T cells in the presence of an increasing concentration of the peptide antigen.

120. The method of claim 109, wherein expanding the third population of T cells in the third cell culture medium comprises expanding the third population of T cells in the presence of a third concentration of the peptide antigen, followed by further expanding the third population of T cells in the presence of a fourth concentration of the peptide antigen, and additionally followed by further expanding at least once the third population of T cells in the presence of at least a fifth concentration of the peptide antigen, wherein the third concentration of the peptide antigen is at least 2-fold lower than the first concentration of the peptide antigen, and wherein the third concentration of the peptide antigen ranges from 0.1 nM to 10 μM.

121. The method of claim 120, wherein the further expanding the third population of T cells in the presence of the fourth concentration of the peptide antigen is in a fourth cell culture medium, and the further expanding at least once the third population in the presence of at least a fifth concentration of the peptide antigen is in a fifth cell culture medium.

122. The method of claim 120, wherein the fourth concentration of the peptide antigen is at least 1.1-fold higher than the third concentration of the peptide antigen and wherein the fifth concentration of the peptide antigen is at least 1.1-fold higher than the fourth concentration of the peptide antigen.

123. The method of claim 120, wherein the fifth concentration of the peptide antigen is at least 2-fold relative to the third concentration of the peptide antigen or the fourth concentration of the peptide antigen.

124. The method of claim 120, wherein the fifth concentration of the peptide antigen is at least 10 nM.

125. The method of claim 109, wherein expanding the third population of T cells in the presence of the third cell culture medium begins at least 11 days after beginning culturing the T cells from the biological sample in the first cell culture medium.

126. The method of claim 120, wherein the further expanding the third population of T cells in the presence of the fourth concentration of the peptide antigen begins at least 1, day after expanding the third population of T cells in the third cell culture medium, and wherein the further expanding the third population of T cells in the presence of the at least fifth concentration of the peptide antigen begins at least 2 days after expanding the third population of T cells in the third cell culture medium.

127. The method of claim 109, wherein frequency of antigen-specific T cells in the ex vivo population of expanded antigen specific T cells is greater than frequency of antigen-specific T cells in the first population of T cells, and wherein the frequency of antigen-specific T cells in the ex vivo population of expanded antigen specific T cells is greater than frequency of antigen-specific T cells in the third population of T cells;
wherein the frequency of antigen-specific T cells is: [number of antigen-specific T cells in the population]/[total number of T cells in the population]×100.

128. The method of claim 126, wherein frequency of antigen-specific T cells in the ex vivo expanded population of antigen specific T cells is greater than the frequency of antigen-specific T cells in the first population of T cells by at least 1.2 fold for the peptide antigen.

129. The method of claim 109, wherein
(i) the ex vivo population of expanded antigen specific T cells or the third population of T cells comprises from 1×10⁷ to 1×10¹¹ total cells,
(ii) the ex vivo population of expanded antigen specific T cells comprises activated CD8+ T cells and CD4+ T cells; and
(iii) obtaining the ex vivo population of expanded antigen specific T cells occurs at or less than 26 days from culturing T cells from the biological sample from the subject in the first cell culture medium.

130. A pharmaceutical composition comprising a therapeutic population of T cells comprising the ex vivo population of expended antigen-specific T cells of claim 109.

131. A method of treating cancer in a subject in need thereof, the method comprising administering to the subject the pharmaceutical composition of claim 130.

132. A method for producing an ex vivo population of expanded antigen specific therapeutic T cells, the method comprising:
a) culturing T cells from a biological sample from a subject in a first cell culture medium comprising antigen presenting cells (APCs), wherein the APCs present an epitope of a peptide antigen in complex with an MEW protein;
b) culturing the first population of T cells in a second cell culture medium to produce a second population of T cells; and
c) expanding the second population of T cells in a third cell culture medium in presence of an increasing concentration of a plurality of peptide antigens comprising the peptide antigen, thereby obtaining the ex vivo population of expanded antigen specific therapeutic T cells.


SEQUENCE LISTING (193 sequences)

SEQ ID NO: 1 - DNA, 30 bp, Artificial Sequence
  Description of Artificial Sequence Synthetic oligonucleotide
ggcggcagcggcggcggcggcagcggcggc

SEQ ID NO: 2 - DNA, 30 bp, Artificial Sequence
  Description of Artificial Sequence Synthetic oligonucleotide
ggcggcagcctgggcggcggcggcagcggc

SEQ ID NO: 3 - DNA, 12 bp, Artificial Sequence
  Description of Artificial Sequence Synthetic oligonucleotide
ggcgtcggcacc

SEQ ID NO: 4 - DNA, 12 bp, Artificial Sequence
  Description of Artificial Sequence Synthetic oligonucleotide
cagctgggcctg

SEQ ID NO: 5 - DNA, 12 bp, Artificial Sequence
  Description of Artificial Sequence Synthetic oligonucleotide
caactgggattg

SEQ ID NO: 6 - protein, 62 aa, Artificial Sequence
  Description of Artificial Sequence Synthetic polypeptide
MTEYKLVVVGACGVGKSALTIQLIQNHFVDEYDPTIEDSYRKQVVIDGETCLLDILDTAGQE

SEQ ID NO: 7 - protein, 62 aa, Artificial Sequence
  Description of Artificial Sequence Synthetic polypeptide
MTEYKLVVVGADGVGKSALTIQLIQNHFVDEYDPTIEDSYRKQVVIDGETCLLDILDTAGQE

SEQ ID NO: 8 - protein, 62 aa, Artificial Sequence
  Description of Artificial Sequence Synthetic polypeptide
MTEYKLVVVGARGVGKSALTIQLIQNHFVDEYDPTIEDSYRKQVVIDGETCLLDILDTAGQE

SEQ ID NO: 9 - protein, 62 aa, Artificial Sequence
  Description of Artificial Sequence Synthetic polypeptide
MTEYKLVVVGAVGVGKSALTIQLIQNHFVDEYDPTIEDSYRKQVVIDGETCLLDILDTAGQE

SEQ ID NO: 10 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGHEEYSAMRDQYMRTGEGFLCVFAINNTKSFEDIHHYREQIKRVKDSEDVPM

SEQ ID NO: 11 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGLEEYSAMRDQYMRTGEGFLCVFAINNTKSFEDIHHYREQIKRVKDSEDVPM

SEQ ID NO: 12 - protein, 9 aa, Artificial Sequence
  Description of Artificial Sequence Synthetic peptide
GADGVGKSA

SEQ ID NO: 13 - protein, 10 aa, Artificial Sequence
  Description of Artificial Sequence Synthetic peptide
GADGVGKSAL

SEQ ID NO: 14 - protein, 10 aa, Artificial Sequence
  Description of Artificial Sequence Synthetic peptide
VVVGADGVGK

SEQ ID NO: 15 - protein, 25 aa, Artificial Sequence
  Description of Artificial Sequence Synthetic peptide
KKKTEYKLVVVGADGVGKSALTIQL

SEQ ID NO: 16 - protein, 9 aa, Artificial Sequence
  Description of Artificial Sequence Synthetic peptide
AVGVGKSAL

SEQ ID NO: 17 - protein, 9 aa, Artificial Sequence
  Description of Artificial Sequence Synthetic peptide
GAVGVGKSA

SEQ ID NO: 18 - protein, 10 aa, Artificial Sequence
  Description of Artificial Sequence Synthetic peptide
GAVGVGKSAL

SEQ ID NO: 19 - protein, 9 aa, Artificial Sequence
  Description of Artificial Sequence Synthetic peptide
VVGAVGVGK

SEQ ID NO: 20 - protein, 10 aa, Artificial Sequence
  Description of Artificial Sequence Synthetic peptide
VVVGAVGVGK

SEQ ID NO: 21 - protein, 26 aa, Artificial Sequence
  Description of Artificial Sequence Synthetic peptide
KKKKTEYKLVVVGAVGVGKSALTIQL

SEQ ID NO: 22 - protein, 10 aa, Artificial Sequence
  Description of Artificial Sequence Synthetic peptide
GARGVGKSAL

SEQ ID NO: 23 - protein, 10 aa, Artificial Sequence
  Description of Artificial Sequence Synthetic peptide
VVVGARGVGK

SEQ ID NO: 24 - protein, 25 aa, Artificial Sequence
  Description of Artificial Sequence Synthetic peptide
KKKTEYKLVVVGARGVGKSALTIQL

SEQ ID NO: 25 - protein, 10 aa, Artificial Sequence
  Description of Artificial Sequence Synthetic peptide
VVVGACGVGK

SEQ ID NO: 26 - protein, 26 aa, Artificial Sequence
  Description of Artificial Sequence Synthetic peptide
KKKKTEYKLVVVGACGVGKSALTIQL

SEQ ID NO: 27 - protein, 10 aa, Artificial Sequence
  Description of Artificial Sequence Synthetic peptide
ILDTAGHEEY

SEQ ID NO: 28 - protein, 11 aa, Artificial Sequence
  Description of Artificial Sequence Synthetic peptide
DTAGHEEYSAM

SEQ ID NO: 29 - protein, 8 aa, Artificial Sequence
  Description of Artificial Sequence Synthetic peptide
GHEEYSAM

SEQ ID NO: 30 - protein, 8 aa, Artificial Sequence
  Description of Artificial Sequence Synthetic peptide
DTAGHEEY

SEQ ID NO: 31 - protein, 9 aa, Artificial Sequence
  Description of Artificial Sequence Synthetic peptide
ILDTAGHEE

SEQ ID NO: 32 - protein, 9 aa, Artificial Sequence
  Description of Artificial Sequence Synthetic peptide
AGHEEYSAM

SEQ ID NO: 33 - protein, 9 aa, Artificial Sequence
  Description of Artificial Sequence Synthetic peptide
HEEYSAMRD

SEQ ID NO: 34 - protein, 9 aa, Artificial Sequence
  Description of Artificial Sequence Synthetic peptide
TAGHEEYSA

SEQ ID NO: 35 - protein, 9 aa, Artificial Sequence
  Description of Artificial Sequence Synthetic peptide
DTAGHEEYS

SEQ ID NO: 36 - protein, 12 aa, Artificial Sequence
  Description of Artificial Sequence Synthetic peptide
DTAGHEEYSAMR

SEQ ID NO: 37 - protein, 9 aa, Artificial Sequence
  Description of Artificial Sequence Synthetic peptide
LDTAGHEEY

SEQ ID NO: 38 - protein, 10 aa, Artificial Sequence
  Description of Artificial Sequence Synthetic peptide
LLDILDTAGH

SEQ ID NO: 39 - protein, 10 aa, Artificial Sequence
  Description of Artificial Sequence Synthetic peptide
TAGHEEYSAM

SEQ ID NO: 40 - protein, 9 aa, Artificial Sequence
  Description of Artificial Sequence Synthetic peptide
DILDTAGHE

SEQ ID NO: 41 - protein, 11 aa, Artificial Sequence
  Description of Artificial Sequence Synthetic peptide
DILDTAGHEEY

SEQ ID NO: 42 - protein, 8 aa, Artificial Sequence
  Description of Artificial Sequence Synthetic peptide
DILDTAGH

SEQ ID NO: 43 - protein, 8 aa, Artificial Sequence
  Description of Artificial Sequence Synthetic peptide
ILDTAGHE

SEQ ID NO: 44 - protein, 10 aa, Artificial Sequence
  Description of Artificial Sequence Synthetic peptide
DTAGHEEYSA

SEQ ID NO: 45 - protein, 10 aa, Artificial Sequence
  Description of Artificial Sequence Synthetic peptide
ILDTAGREEY

SEQ ID NO: 46 - protein, 11 aa, Artificial Sequence
  Description of Artificial Sequence Synthetic peptide
DTAGREEYSAM

SEQ ID NO: 47 - protein, 8 aa, Artificial Sequence
  Description of Artificial Sequence Synthetic peptide
DILDTAGR

SEQ ID NO: 48 - protein, 8 aa, Artificial Sequence
  Description of Artificial Sequence Synthetic peptide
DTAGREEY

SEQ ID NO: 49 - protein, 11 aa, Artificial Sequence
  Description of Artificial Sequence Synthetic peptide
CLLDILDTAGR

SEQ ID NO: 50 - protein, 9 aa, Artificial Sequence
  Description of Artificial Sequence Synthetic peptide
REEYSAMRD

SEQ ID NO: 51 - protein, 9 aa, Artificial Sequence
  Description of Artificial Sequence Synthetic peptide
GREEYSAMR

SEQ ID NO: 52 - protein, 9 aa, Artificial Sequence
  Description of Artificial Sequence Synthetic peptide
ILDTAGREE

SEQ ID NO: 53 - protein, 9 aa, Artificial Sequence
  Description of Artificial Sequence Synthetic peptide
AGREEYSAM

SEQ ID NO: 54 - protein, 9 aa, Artificial Sequence
  Description of Artificial Sequence Synthetic peptide
DILDTAGRE

SEQ ID NO: 55 - protein, 8 aa, Artificial Sequence
  Description of Artificial Sequence Synthetic peptide
GREEYSAM

SEQ ID NO: 56 - protein, 9 aa, Artificial Sequence
  Description of Artificial Sequence Synthetic peptide
TAGREEYSA

SEQ ID NO: 57 - protein, 9 aa, Artificial Sequence
  Description of Artificial Sequence Synthetic peptide
DTAGREEYS

SEQ ID NO: 58 - protein, 8 aa, Artificial Sequence
  Description of Artificial Sequence Synthetic peptide
ILDTAGRE

SEQ ID NO: 59 - protein, 12 aa, Artificial Sequence
  Description of Artificial Sequence Synthetic peptide
DTAGREEYSAMR

SEQ ID NO: 60 - protein, 10 aa, Artificial Sequence
  Description of Artificial Sequence Synthetic peptide
LLDILDTAGR

SEQ ID NO: 61 - protein, 10 aa, Artificial Sequence
  Description of Artificial Sequence Synthetic peptide
TAGREEYSAM

SEQ ID NO: 62 - protein, 11 aa, Artificial Sequence
  Description of Artificial Sequence Synthetic peptide
DILDTAGREEY

SEQ ID NO: 63 - protein, 10 aa, Artificial Sequence
  Description of Artificial Sequence Synthetic peptide
AGREEYSAMR

SEQ ID NO: 64 - protein, 9 aa, Artificial Sequence
  Description of Artificial Sequence Synthetic peptide
LDILDTAGR

SEQ ID NO: 65 - protein, 9 aa, Artificial Sequence
  Description of Artificial Sequence Synthetic peptide
LDTAGREEY

SEQ ID NO: 66 - protein, 11 aa, Artificial Sequence
  Description of Artificial Sequence Synthetic peptide
REEYSAMRDQY

SEQ ID NO: 67 - protein, 10 aa, Artificial Sequence
  Description of Artificial Sequence Synthetic peptide
ILDTAGKEEY

SEQ ID NO: 68 - protein, 11 aa, Artificial Sequence
  Description of Artificial Sequence Synthetic peptide
DTAGKEEYSAM

SEQ ID NO: 69 - protein, 11 aa, Artificial Sequence
  Description of Artificial Sequence Synthetic peptide
CLLDILDTAGK

SEQ ID NO: 70 - protein, 8 aa, Artificial Sequence
  Description of Artificial Sequence Synthetic peptide
DTAGKEEY

SEQ ID NO: 71 - protein, 9 aa, Artificial Sequence
  Description of Artificial Sequence Synthetic peptide
AGKEEYSAM

SEQ ID NO: 72 - protein, 9 aa, Artificial Sequence
  Description of Artificial Sequence Synthetic peptide
DILDTAGKE

SEQ ID NO: 73 - protein, 9 aa, Artificial Sequence
  Description of Artificial Sequence Synthetic peptide
KEEYSAMRD

SEQ ID NO: 74 - protein, 8 aa, Artificial Sequence
  Description of Artificial Sequence Synthetic peptide
GKEEYSAM

SEQ ID NO: 75 - protein, 9 aa, Artificial Sequence
  Description of Artificial Sequence Synthetic peptide
ILDTAGKEE

SEQ ID NO: 76 - protein, 9 aa, Artificial Sequence
  Description of Artificial Sequence Synthetic peptide
DTAGKEEYS

SEQ ID NO: 77 - protein, 9 aa, Artificial Sequence
  Description of Artificial Sequence Synthetic peptide
LDTAGKEEY

SEQ ID NO: 78 - protein, 9 aa, Artificial Sequence
  Description of Artificial Sequence Synthetic peptide
TAGKEEYSA

SEQ ID NO: 79 - protein, 8 aa, Artificial Sequence
  Description of Artificial Sequence Synthetic peptide
DILDTAGK

SEQ ID NO: 80 - protein, 12 aa, Artificial Sequence
  Description of Artificial Sequence Synthetic peptide
DTAGKEEYSAMR

SEQ ID NO: 81 - protein, 10 aa, Artificial Sequence
  Description of Artificial Sequence Synthetic peptide
LLDILDTAGK

SEQ ID NO: 82 - protein, 8 aa, Artificial Sequence
  Description of Artificial Sequence Synthetic peptide
ILDTAGKE

SEQ ID NO: 83 - protein, 10 aa, Artificial Sequence
  Description of Artificial Sequence Synthetic peptide
AGKEEYSAMR

SEQ ID NO: 84 - protein, 10 aa, Artificial Sequence
  Description of Artificial Sequence Synthetic peptide
TAGKEEYSAM

SEQ ID NO: 85 - protein, 10 aa, Artificial Sequence
  Description of Artificial Sequence Synthetic peptide
ILDTAGLEEY

SEQ ID NO: 86 - protein, 10 aa, Artificial Sequence
  Description of Artificial Sequence Synthetic peptide
LLDILDTAGL

SEQ ID NO: 87 - protein, 12 aa, Artificial Sequence
  Description of Artificial Sequence Synthetic peptide
GLEEYSAMRDQY

SEQ ID NO: 88 - protein, 8 aa, Artificial Sequence
  Description of Artificial Sequence Synthetic peptide
DTAGLEEY

SEQ ID NO: 89 - protein, 11 aa, Artificial Sequence
  Description of Artificial Sequence Synthetic peptide
DTAGLEEYSAM

SEQ ID NO: 90 - protein, 9 aa, Artificial Sequence
  Description of Artificial Sequence Synthetic peptide
ILDTAGLEE

SEQ ID NO: 91 - protein, 11 aa, Artificial Sequence
  Description of Artificial Sequence Synthetic peptide
CLLDILDTAGL

SEQ ID NO: 92 - protein, 8 aa, Artificial Sequence
  Description of Artificial Sequence Synthetic peptide
DILDTAGL

SEQ ID NO: 93 - protein, 11 aa, Artificial Sequence
  Description of Artificial Sequence Synthetic peptide
DILDTAGLEEY

SEQ ID NO: 94 - protein, 9 aa, Artificial Sequence
  Description of Artificial Sequence Synthetic peptide
DTAGLEEYS

SEQ ID NO: 95 - protein, 9 aa, Artificial Sequence
  Description of Artificial Sequence Synthetic peptide
GLEEYSAMR

SEQ ID NO: 96 - protein, 8 aa, Artificial Sequence
  Description of Artificial Sequence Synthetic peptide
ILDTAGLE

SEQ ID NO: 97 - protein, 9 aa, Artificial Sequence
  Description of Artificial Sequence Synthetic peptide
LDTAGLEEY

SEQ ID NO: 98 - protein, 12 aa, Artificial Sequence
  Description of Artificial Sequence Synthetic peptide
DTAGLEEYSAMR

SEQ ID NO: 99 - protein, 9 aa, Artificial Sequence
  Description of Artificial Sequence Synthetic peptide
AGLEEYSAM

SEQ ID NO: 100 - protein, 10 aa, Artificial Sequence
  Description of Artificial Sequence Synthetic peptide
DTAGLEEYSA

SEQ ID NO: 101 - protein, 9 aa, Artificial Sequence
  Description of Artificial Sequence Synthetic peptide
LDILDTAGL

SEQ ID NO: 102 - protein, 10 aa, Artificial Sequence
  Description of Artificial Sequence Synthetic peptide
AGLEEYSAMR

SEQ ID NO: 103 - protein, 9 aa, Artificial Sequence
  Description of Artificial Sequence Synthetic peptide
LEEYSAMRD

SEQ ID NO: 104 - protein, 11 aa, Artificial Sequence
  Description of Artificial Sequence Synthetic peptide
LLDILDTAGLE

SEQ ID NO: 105 - protein, 9 aa, Artificial Sequence
  Description of Artificial Sequence Synthetic peptide
DILDTAGLE

SEQ ID NO: 106 - protein, 9 aa, Artificial Sequence
  Description of Artificial Sequence Synthetic peptide
TAGLEEYSA

SEQ ID NO: 107 - protein, 8 aa, Artificial Sequence
  Description of Artificial Sequence Synthetic peptide
GLEEYSAM

SEQ ID NO: 108 - protein, 9 aa, Artificial Sequence
  Description of Artificial Sequence Synthetic peptide
AAGVGKSAL

SEQ ID NO: 109 - protein, 10 aa, Artificial Sequence
  Description of Artificial Sequence Synthetic peptide
VVVGAAGVGK

SEQ ID NO: 110 - protein, 9 aa, Artificial Sequence
  Description of Artificial Sequence Synthetic peptide
VVGAAGVGK

SEQ ID NO: 111 - protein, 11 aa, Artificial Sequence
  Description of Artificial Sequence Synthetic peptide
TEYKLVVVGAA

SEQ ID NO: 112 - protein, 10 aa, Artificial Sequence
  Description of Artificial Sequence Synthetic peptide
GAAGVGKSAL

SEQ ID NO: 113 - protein, 9 aa, Artificial Sequence
  Description of Artificial Sequence Synthetic peptide
LVVVGAAGV

SEQ ID NO: 114 - protein, 9 aa, Artificial Sequence
  Description of Artificial Sequence Synthetic peptide
GAAGVGKSA

SEQ ID NO: 115 - protein, 10 aa, Artificial Sequence
  Description of Artificial Sequence Synthetic peptide
KLVVVGAAGV

SEQ ID NO: 116 - protein, 8 aa, Artificial Sequence
  Description of Artificial Sequence Synthetic peptide
AGVGKSAL

SEQ ID NO: 117 - protein, 8 aa, Artificial Sequence
  Description of Artificial Sequence Synthetic peptide
VVVGAAGV

SEQ ID NO: 118 - protein, 9 aa, Artificial Sequence
  Description of Artificial Sequence Synthetic peptide
YKLVVVGAA

SEQ ID NO: 119 - protein, 8 aa, Artificial Sequence
  Description of Artificial Sequence Synthetic peptide
AAGVGKSA

SEQ ID NO: 120 - protein, 10 aa, Artificial Sequence
  Description of Artificial Sequence Synthetic peptide
VGAAGVGKSA

SEQ ID NO: 121 - protein, 10 aa, Artificial Sequence
  Description of Artificial Sequence Synthetic peptide
AGVGKSALTI

SEQ ID NO: 122 - protein, 9 aa, Artificial Sequence
  Description of Artificial Sequence Synthetic peptide
VVGACGVGK

SEQ ID NO: 123 - protein, 9 aa, Artificial Sequence
  Description of Artificial Sequence Synthetic peptide
ACGVGKSAL

SEQ ID NO: 124 - protein, 8 aa, Artificial Sequence
  Description of Artificial Sequence Synthetic peptide
CGVGKSAL

SEQ ID NO: 125 - protein, 10 aa, Artificial Sequence
  Description of Artificial Sequence Synthetic peptide
KLVVVGACGV

SEQ ID NO: 126 - protein, 10 aa, Artificial Sequence
  Description of Artificial Sequence Synthetic peptide
GACGVGKSAL

SEQ ID NO: 127 - protein, 9 aa, Artificial Sequence
  Description of Artificial Sequence Synthetic peptide
YKLVVVGAC

SEQ ID NO: 128 - protein, 9 aa, Artificial Sequence
  Description of Artificial Sequence Synthetic peptide
GACGVGKSA

SEQ ID NO: 129 - protein, 9 aa, Artificial Sequence
  Description of Artificial Sequence Synthetic peptide
LVVVGACGV

SEQ ID NO: 130 - protein, 8 aa, Artificial Sequence
  Description of Artificial Sequence Synthetic peptide
DGVGKSAL

SEQ ID NO: 131 - protein, 9 aa, Artificial Sequence
  Description of Artificial Sequence Synthetic peptide
VVGADGVGK

SEQ ID NO: 132 - protein, 9 aa, Artificial Sequence
  Description of Artificial Sequence Synthetic peptide
ADGVGKSAL

SEQ ID NO: 133 - protein, 8 aa, Artificial Sequence
  Description of Artificial Sequence Synthetic peptide
GADGVGKS

SEQ ID NO: 134 - protein, 10 aa, Artificial Sequence
  Description of Artificial Sequence Synthetic peptide
KLVVVGADGV

SEQ ID NO: 135 - protein, 9 aa, Artificial Sequence
  Description of Artificial Sequence Synthetic peptide
LVVVGADGV

SEQ ID NO: 136 - protein, 10 aa, Artificial Sequence
  Description of Artificial Sequence Synthetic peptide
VGADGVGKSA

SEQ ID NO: 137 - protein, 8 aa, Artificial Sequence
  Description of Artificial Sequence Synthetic peptide
VGADGVGK

SEQ ID NO: 138 - protein, 9 aa, Artificial Sequence
  Description of Artificial Sequence Synthetic peptide
YKLVVVGAD

SEQ ID NO: 139 - protein, 10 aa, Artificial Sequence
  Description of Artificial Sequence Synthetic peptide
DGVGKSALTI

SEQ ID NO: 140 - protein, 9 aa, Artificial Sequence
  Description of Artificial Sequence Synthetic peptide
VVGARGVGK

SEQ ID NO: 141 - protein, 10 aa, Artificial Sequence
  Description of Artificial Sequence Synthetic peptide
EYKLVVVGAR

SEQ ID NO: 142 - protein, 9 aa, Artificial Sequence
  Description of Artificial Sequence Synthetic peptide
ARGVGKSAL

SEQ ID NO: 143 - protein, 9 aa, Artificial Sequence
  Description of Artificial Sequence Synthetic peptide
GARGVGKSA

SEQ ID NO: 144 - protein, 8 aa, Artificial Sequence
  Description of Artificial Sequence Synthetic peptide
RGVGKSAL

SEQ ID NO: 145 - protein, 10 aa, Artificial Sequence
  Description of Artificial Sequence Synthetic peptide
RGVGKSALTI

SEQ ID NO: 146 - protein, 9 aa, Artificial Sequence
  Description of Artificial Sequence Synthetic peptide
LVVVGARGV

SEQ ID NO: 147 - protein, 10 aa, Artificial Sequence
  Description of Artificial Sequence Synthetic peptide
KLVVVGARGV

SEQ ID NO: 148 - protein, 9 aa, Artificial Sequence
  Description of Artificial Sequence Synthetic peptide
YKLVVVGAR

SEQ ID NO: 149 - protein, 8 aa, Artificial Sequence
  Description of Artificial Sequence Synthetic peptide
KLVVVGAR

SEQ ID NO: 150 - protein, 8 aa, Artificial Sequence
  Description of Artificial Sequence Synthetic peptide
VVVGARGV

SEQ ID NO: 151 - protein, 10 aa, Artificial Sequence
  Description of Artificial Sequence Synthetic peptide
VVVGASGVGK

SEQ ID NO: 152 - protein, 9 aa, Artificial Sequence
  Description of Artificial Sequence Synthetic peptide
VVGASGVGK

SEQ ID NO: 153 - protein, 9 aa, Artificial Sequence
  Description of Artificial Sequence Synthetic peptide
ASGVGKSAL

SEQ ID NO: 154 - protein, 10 aa, Artificial Sequence
  Description of Artificial Sequence Synthetic peptide
GASGVGKSAL

SEQ ID NO: 155 - protein, 8 aa, Artificial Sequence
  Description of Artificial Sequence Synthetic peptide
SGVGKSAL

SEQ ID NO: 156 - protein, 9 aa, Artificial Sequence
  Description of Artificial Sequence Synthetic peptide
GASGVGKSA

SEQ ID NO: 157 - protein, 10 aa, Artificial Sequence
  Description of Artificial Sequence Synthetic peptide
KLVVVGASGV

SEQ ID NO: 158 - protein, 9 aa, Artificial Sequence
  Description of Artificial Sequence Synthetic peptide
LVVVGASGV

SEQ ID NO: 159 - protein, 8 aa, Artificial Sequence
  Description of Artificial Sequence Synthetic peptide
ASGVGKSA

SEQ ID NO: 160 - protein, 10 aa, Artificial Sequence
  Description of Artificial Sequence Synthetic peptide
SGVGKSALTI

SEQ ID NO: 161 - protein, 10 aa, Artificial Sequence
  Description of Artificial Sequence Synthetic peptide
VGASGVGKSA

SEQ ID NO: 162 - protein, 9 aa, Artificial Sequence
  Description of Artificial Sequence Synthetic peptide
KLVVVGASG

SEQ ID NO: 163 - protein, 9 aa, Artificial Sequence
  Description of Artificial Sequence Synthetic peptide
LVVVGAVGV

SEQ ID NO: 164 - protein, 10 aa, Artificial Sequence
  Description of Artificial Sequence Synthetic peptide
KLVVVGAVGV

SEQ ID NO: 165 - protein, 8 aa, Artificial Sequence
  Description of Artificial Sequence Synthetic peptide
VGVGKSAL

SEQ ID NO: 166 - protein, 8 aa, Artificial Sequence
  Description of Artificial Sequence Synthetic peptide
VVVGAVGV

SEQ ID NO: 167 - protein, 10 aa, Artificial Sequence
  Description of Artificial Sequence Synthetic peptide
VGVGKSALTI

SEQ ID NO: 168 - protein, 8 aa, Artificial Sequence
  Description of Artificial Sequence Synthetic peptide
AVGVGKSA

SEQ ID NO: 169 - protein, 9 aa, Artificial Sequence
  Description of Artificial Sequence Synthetic peptide
YKLVVVGAV

SEQ ID NO: 170 - protein, 10 aa, Artificial Sequence
  Description of Artificial Sequence Synthetic peptide
VVVGAGCVGK

SEQ ID NO: 171 - protein, 9 aa, Artificial Sequence
  Description of Artificial Sequence Synthetic peptide
VVGAGCVGK

SEQ ID NO: 172 - protein, 9 aa, Artificial Sequence
  Description of Artificial Sequence Synthetic peptide
AGCVGKSAL

SEQ ID NO: 173 - protein, 9 aa, Artificial Sequence
  Description of Artificial Sequence Synthetic peptide
CVGKSALTI

SEQ ID NO: 174 - protein, 10 aa, Artificial Sequence
  Description of Artificial Sequence Synthetic peptide
GCVGKSALTI

SEQ ID NO: 175 - protein, 9 aa, Artificial Sequence
  Description of Artificial Sequence Synthetic peptide
KLVVVGAGC

SEQ ID NO: 176 - protein, 8 aa, Artificial Sequence
  Description of Artificial Sequence Synthetic peptide
GCVGKSAL

SEQ ID NO: 177 - protein, 9 aa, Artificial Sequence
  Description of Artificial Sequence Synthetic peptide
AGDVGKSAL

SEQ ID NO: 178 - protein, 9 aa, Artificial Sequence
  Description of Artificial Sequence Synthetic peptide
VVGAGDVGK

SEQ ID NO: 179 - protein, 10 aa, Artificial Sequence
  Description of Artificial Sequence Synthetic peptide
VVVGAGDVGK

SEQ ID NO: 180 - protein, 9 aa, Artificial Sequence
  Description of Artificial Sequence Synthetic peptide
GAGDVGKSA

SEQ ID NO: 181 - protein, 10 aa, Artificial Sequence
  Description of Artificial Sequence Synthetic peptide
GAGDVGKSAL

SEQ ID NO: 182 - protein, 9 aa, Artificial Sequence
  Description of Artificial Sequence Synthetic peptide
DVGKSALTI

SEQ ID NO: 183 - protein, 8 aa, Artificial Sequence
  Description of Artificial Sequence Synthetic peptide
GDVGKSAL

SEQ ID NO: 184 - RNA, 200 nt, Artificial Sequence
  Description of Artificial Sequence Synthetic polynucleotide
  misc_feature (1)..(200): This sequence may encompass 100-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 185 - RNA, 250 nt, Artificial Sequence
  Description of Artificial Sequence Synthetic polynucleotide
  misc_feature (1)..(250): This sequence may encompass 100-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 186 - RNA, 130 nt, Artificial Sequence
  Description of Artificial Sequence Synthetic polynucleotide
  misc_feature (1)..(200): This sequence may encompass 120-130 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 187 - RNA, 120 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 188 - RNA, 121 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 189 - RNA, 122 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 190 - RNA, 123 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 191 - RNA, 124 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 192 - RNA, 125 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 193 - RNA, 129 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa